(19) United States
(12) Patent Application Publication
(10) Pub. No.: US 2013/0053372 A1
(43) Pub. Date: Feb. 28, 2013

(54) 5-HT RECEPTOR MODULATORS

(71) Applicants: Prashant Bhimrao Kapadnis, Cambridge (GB); Robert Glen, Cambridge (GB); Robin Hiley, Cambridge (GB); James Bell, Cambridge (GB); David Spring, Cambridge (GB)

(72) Inventors: Prashant Bhimrao Kapadnis, Cambridge (GB); Robert Glen, Cambridge (GB); Robin Hiley, Cambridge (GB); James Bell, Cambridge (GB); David Spring, Cambridge (GB)

(73) Assignee: Cambridge Enterprise Limited

(21) Appl. No.: 13/578,796

(22) PCT Filed: Feb. 15, 2011
(86) PCT No.: PCT/GB2011/000204

§ 371 Date: Nov. 14, 2012

(30) Foreign Application Priority Data
Feb. 15, 2010 (GB) 1002563.3

(51) Int. Cl. A61K 31/496; C07D 413/10; A61K 31/551; C07D 417/10; C07D 405/14; C07D 413/14; C07D 491/04; C07D 401/14; A61P 35/00; A61P 9/00; A61P 25/00; A61P 9/10; A61P 9/12; A61P 1/00; C07D 403/10

(52) U.S. Cl. 514/218; 544/372; 514/254.01; 544/369; 514/254.02; 540/575; 544/367; 514/254.04; 514/254.03; 544/362; 514/253.04; 544/364; 514/253.09

(57) ABSTRACT

The invention relates to compounds of formula (I), useful for treating disorders mediated by the 5-hydroxytryptamine (serotonin) receptor IB (5-HT1B), e.g. vascular disorders, cancer and CNS disorders. The invention also provides methods of treating such disorders, and compounds and compositions etc. for their treatment.

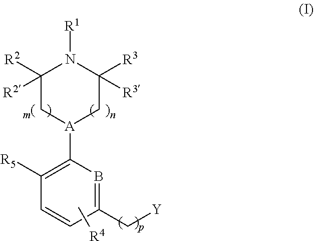

### TECHNICAL FIELD

This invention relates to compounds useful for treating disorders mediated by the 5-hydroxytryptamine (serotonin) receptor 1B (5-HT_{1B}). The invention also provides methods of treating such disorders, and compounds and compositions etc. for their treatment.

### BACKGROUND ART

Serotonin (5-HT) has been implicated in cardiovascular and hemostatic regulation, blood pressure regulation, arterial and venous tone, blood clotting, motor disorders, endocrine disorders, vasospasm, sexual dysfunction, gastrointestinal disorders and chronic obstructive pulmonary disease (COPD). 5-HT has also been implicated in many central nervous system and psychiatric disorders, including depression, generalized anxiety, eating disorders, dementia, panic disorder and sleep disorders.

Serotonin receptors have been subdivided into at least 14 subtypes (see Barnes and Sharp, Neuropharmacology, 1999, 38, 1083-1152). These various subtypes are responsible for serotonin's action in many pathophysiological conditions. The 5-HT₁ family of receptors has high affinity for serotonin and comprises five receptor subtypes, 5-HT_{1A}, 5-HT_{1B}, 5-HT_{1D}, 5-HT_{1E} and 5-HT_{1F}.

Compounds that interact with the 5-HT₁ families are known to have therapeutic potential in the above disorders and diseases. In particular, compounds that are 5-HT_{1B} receptor antagonists have been known to be antidepressant and anxiolytic agents and useful for treating gastrointestinal disorders, vasospasm, angina and COPD.

It has also been found that 5-HT_{1B} receptors are present in smooth muscle. Consequently, it is expected that compounds which exhibit 5-HT_{1B} receptor antagonist activity will be useful in treating vascular disease such as angina, Raynaud's syndrome, peripheral vascular disease and portal hypertension (U.S. Pat. No. 6,107,328). The 5-HT_{1B} receptor has also been found to be a promising target for the treatment of cancer, in particular, bladder and prostate cancer (see BJU Int 2006, 97(3), 634-9 and J Urol. 2006, 176(4 Pt 1), 1648-53).

There is therefore a need for compounds which modulate 5-HT_{1B} receptors.

WO 99/05134 describes piperidyl- or piperazinyl-substituted 1,2,3,4-tetrahydronaphthalene derivatives useful as 5-HT_{1B} receptor antagonists.

WO 99/14207 describes piperazinyl-substituted indane derivatives useful as 5-HT_{1B} receptor antagonists.

WO 99/02502 describes aryl piperazine sulphonamide derivatives selective for the 5-HT₆ receptor for the treatment of anxiety and depression.

WO 2006/010629 describes aryl piperidine sulphonamide derivatives having selective agonistic activity at the growth hormone secretagogue (GHS) receptors and useful in treating gastrointestinal disorders.

WO 95/11243 describes piperazine substituted benzo-2,3-dihydrofuran derivatives useful as 5-HT_{1D} receptor antagonists.

U.S. Pat. No. 6,107,328 describes tetrahydrospiroindolinenes as 5-HT_{1B} receptor antagonists useful in treating angina, Raynaud's syndrome, peripheral vascular disease and portal hypertension.

The compounds of the present invention are 5-HT_{1B} receptor modulators useful in treating disorders including, but not limited to, those disclosed above.

### DISCLOSURE OF THE INVENTION The inventors have found compounds of formula (I) that are useful for modulating the 5-HT_{1B} receptor.

In a first aspect of the invention, there is provided a compound of formula (I):

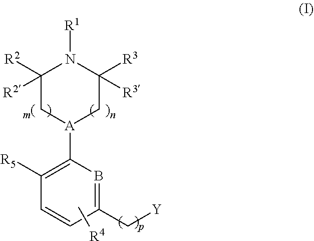

or a pharmaceutically acceptable derivative thereof,
wherein:
A and B are each independently selected from CH and N;
m is 0, 1 or 2;
n is 0, 1 or 2;
p is 0, 1 or 2;
R¹ is H or optionally substituted C₁₋₁₀alkyl, C₃₋₁₀cycloalkyl, C₁-C₁₁heteroalkyl, C₃₋₁₀heterocycloalkyl, C₆₋₁₄aryl or C₅₋₁₄heteroaryl;
R² and R²′ are each independently selected from H and optionally substituted C₁₋₁₀alkyl or C₃₋₁₀cycloalkyl;
R³ and R³′ are each independently selected from H and optionally substituted C₁₋₁₀alkyl or C₃₋₁₀ocycloalkyl;
R⁴ is H, NH₂, NO₂, halo, CN or optionally substituted C₁₋₁₀alkyl, C₁₋₁₁heteroalkyl, C₆₋₁₄aryl or C₅₋₁₄heteroaryl;
R⁵ is H, NH₂, NO₂, halo, CN or optionally substituted C₁₋₁₀alkyl, C₁₋₁₁heteroalkyl, C₆₋₁₄aryl or C₅₋₁₄heteroaryl; or R⁵ is taken together with the carbon atom to which it is attached and the adjacent carbon atom to form a 5- or 6-membered ring in a compound according to formula (Ia) or (Ib):

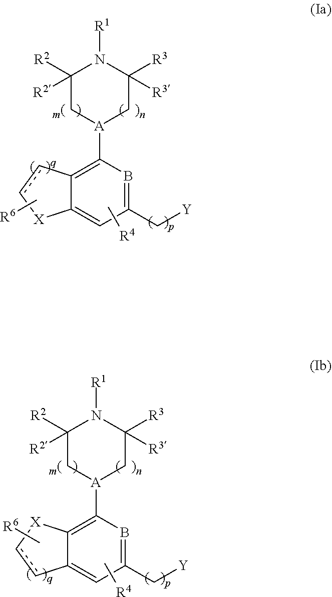

wherein,
   X is CH₂, NH, NC₁₋₁₀alkyl, NC(O)C₁₋₁₀alkyl, O or S;
   R⁶ is H, NH₂, NO₂, halo, CN or optionally substituted C₁₋₁₀alkyl, C₁₋₁₁heteroalkyl, C₆₋₁₄aryl or C₅₋₁₄heteroaryl;
   q is 1 or 2; and
Y is optionally substituted C₃₋₁₀heterocycloalkyl, C₅₋₁₀heterocycloalkenyl or C₅₋₁₄heteroaryl.

In another aspect of the invention there is provided a compound of formula (1):

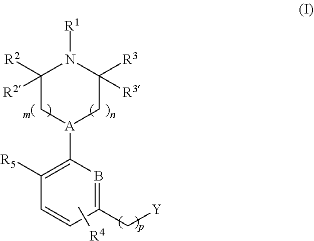

or a pharmaceutically acceptable derivative thereof,
wherein:
A and B are each independently selected from CH and N;
m is 0, 1 or 2;
n is 0, 1 or 2;
p is 0, 1 or 2;
R¹ is H or optionally substituted C₁₋₁₀alkyl, C₃₋₁₀cycloalkyl, C₁-C₁₁heteroalkyl, C₃₋₁₀heterocycloalkyl, C₆₋₁₄aryl or C₅₋₁₄heteroaryl;
R² and R²′ are each independently selected from H and optionally substituted C₁₋₁₀alkyl or C₃₋₁₀cycloalkyl;
R³ and R³′ are each independently selected from H and optionally substituted C₁₋₁₀alkyl or C₃₋₁₀cycloalkyl;
R⁴ is H, halo, CN or optionally substituted C₁₋₁₀alkyl, C₁₋₁₁heteroalkyl, C₆₋₁₄aryl or C₅₋₁₄heteroaryl;
R⁵ is H, halo, CN or optionally substituted C₁₋₁₀alkyl, C₁₋₁₁heteroalkyl, C₆₋₁₄aryl or C₅₋₁₄heteroaryl; or R⁵ is taken together with the carbon atom to which it is attached and the adjacent carbon atom to form a 5- or 6-membered ring in a compound according to formula (Ia) or (Ib):

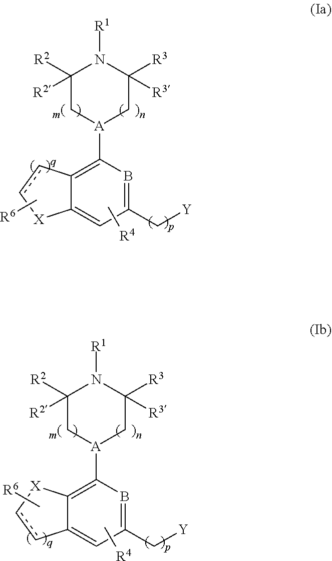

wherein,
   X is CH₂, NH, NC₁₋₁₀alkyl, NC(O)C₁₋₁₀alkyl, O or S;
R⁶ is H, halo, CN or optionally substituted C₁₋₁₀alkyl, C₁₋₁₁heteroalkyl, C₆₋₁₄aryl or C₅₋₁₄heteroaryl;
   q is 1 or 2; and
Y is optionally substituted C₃₋₁₀heterocycloalkyl, C₅₋₁₀heterocycloalkenyl or C₅₋₁₄heteroaryl.

In a particular embodiment of the invention, there is provided a compound of formula (I):

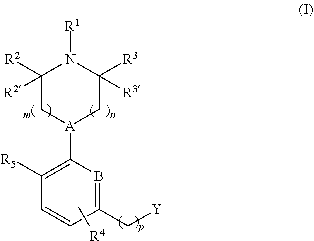

or a pharmaceutically acceptable derivative thereof,
wherein:
A and B are each independently selected from CH and N;
m is 0, 1 or 2;
n is 0, 1 or 2;
p is 0, 1 or 2;
R¹ is H or optionally substituted C₁₋₁₀alkyl, C₃₋₁₀cycloalkyl, C₁-C₁heteroalkyl, C₃₋₁₀heterocycloalkyl, C₆₋₁₄aryl or C₅₋₁₄heteroaryl;
R² and R²′ are each independently selected from H and optionally substituted C₁₋₁₀alkyl or C₃₋₁₀ocycloalkyl;
R³ and R³′ are each independently selected from H and optionally substituted C₁₋₁₀alkyl or C₃₋₁₀cycloalkyl;
R⁴ is H, NH₂, NO₂, halo, CN or optionally substituted C₁₋₁₀alkyl, C₁₋₁₁heteroalkyl, C₆₋₁₄aryl or C₅₋₁₄heteroaryl;
R⁵ is H, NH₂, NO₂, halo, CN or optionally substituted C₁₋₁₀alkyl, C₁₋₁₁heteroalkyl, C₆₋₁₄aryl or C₅₋₁₄heteroaryl; or R⁵ is taken together with the carbon atom to which it is attached and the adjacent carbon atom to form a 5- or 6-membered ring in a compound according to formula (Ia) or (Ib):

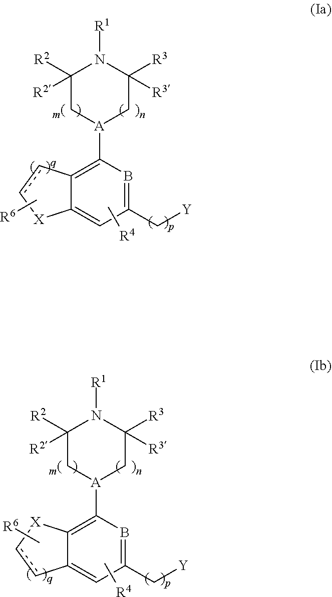

wherein,
   X is CH₂, NH, NC₁₋₁₀alkyl, NC(O)C₁₋₁₀alkyl, O or S;
   R⁶ is H, NH₂, NO₂, halo, CN or optionally substituted C₁₋₁₀alkyl, C₁₋₁₁heteroalkyl, C₆₋₁₄aryl or C₅₋₁₄heteroaryl;
   q is 1 or 2; and
Y is C₃₋₁₀heterocycloalkyl, C₅₋₁₀heterocycloalkenyl or C₅₋₁₄heteroaryl each optionally substituted with one or more substituents independently selected from the group consisting of halogen, trihalomethyl, trihaloethyl, —NO₂, —CN, —N⁺(C₁₋₆alkyl)₂O⁻, —CO₂H, —CO₂C₁₋₆alkyl, —SO₃H, —SOC₁₋₆alkyl, —SO₂C₁₋₆alkyl, —SO₃C₁₋₆alkyl, —OC(═O)OC₁₋₆alkyl, —C(—O)H, —C(═O)C₁₋₆alkyl, —OC(═O)C₁₋₆alkyl, ═O, —(C₁₋₆alkyl)₂, —C(═O)NH₂, —C(═O)N(C₁₋₆alkyl)₂, —N(C₁₋₆alkyl)C(═O)P(C₁₋₆alkyl), —N(C₁₋₆alkyl)C(═O)N(C₁₋₆alkyl)₂, —OC(═O)N(C₁₋₆alkyl)₂, —N(C₁₋₆alkyl)C(═O)C₁₋₆alkyl, —C(═S)N(C₁₋₆alkyl)₂, —N(C₁₋₆alkyl)C(═S)C₁₋₆alkyl, —SO₂N(C₁₋₆alkyl)₂, —N(C₁₋₆alkyl)SO₂C₁₋₆alkyl, —N(C₁₋₆alkyl)C(═S)N(C₁₋₆alkyl)₂, —N(C₁₋₆alkyl)SO₂N(C₁₋₆alkyl)₂, and optionally substituted C₁₋₁₀alkyl, C₁₋₁₁heteroalkyl, C₃₋₁₀cycloalkyl, C₃₋₁₀heterocycloalkyl, C₂₋₆alkenyl, C₂₋₆heteroalkenyl, C₃₋₆cycloalkenyl, C₅₋₁₀heterocycloalkenyl, C₂₋₆alkynyl, C₂₋₆heteroalkynyl, C₆₋₁₄aryl, C₆₋₁₄heteroaryl, —Z^{u}—C₁₋₆alkyl, —Z^{u}—C₃₋₆cycloalkyl, —Z^{u}—C₂₋₆alkenyl, —Z^{u}—C₃₋₆cycloalkenyl and —Z^{u}—C₂₋₆alkynyl; wherein two adjacent substituents taken together with the C or N atoms of the Y group to which they are attached may form an optionally substituted C₆₋₁₄aryl or C₅₋₁₄heteroaryl moiety; and wherein
   Z^{u} is independently O, S, NH or N(C₁₋₆alkyl).

In a particular embodiment of the compounds of formula (I), Y is selected from:

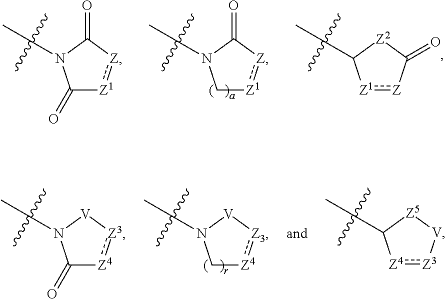

wherein
   a and r are independently 0, 1, 2 or 3;
   Z is CR⁷ or C(R⁷)₂ and Z¹ is CR⁸ or C(R⁸)₂, or
   Z is CR⁷ or C(R⁷)₂ and Z¹ is N, NR⁸, O, or S, or
   Z is N, NR⁷, O or S and Z¹ is CR⁸ or C(R⁸)₂, wherein
      each R⁷ and R⁸ is independently selected from H and optionally substituted C₁₋₁₀alkyl, C₁₁₁heteroalkyl, C₃₋₁₀cycloalkyl, C₃₋₁₀heterocycloalkyl, C₅₋₁₀heterocycloalkenyl, C₆₋₁₄aryl and C₅₋₁₄heteroaryl; or R⁷ and R⁸ are taken together with the C or N atoms to which they are attached to form an optionally substituted C₆₋₁₄aryl or C₅₋₁₄heteroaryl moiety;
   Z² is CH₂, NH, O or S;
   V is S(O)_{y}, wherein
      y is 1 or 2;
   Z³ is CR⁹ or C(R⁹)₂ and Z⁴ is CR¹⁰or C(R¹⁰)₂, or
   Z³ is CR⁹ or C(R⁹)₂ and Z⁴ is N, NR¹⁰ or O, or
   Z³ is N, NR⁹ or O and Z⁴ is CR¹⁰ or C(R¹⁰)₂, wherein
      each R⁹ and R¹⁰ is independently selected from H and optionally substituted C₁₋₁₀alkyl, C₁₋₁₁heteroalkyl, C₃₋₁₀cycloalkyl, C₃₋₁₀heterocycloalkyl, C₅₋₁₀heterocycloalkenyl, C₆₋₁₄aryl and C₅₋₁₄heteroaryl; or R⁹ and R¹⁰ are taken together with the C or N atoms to which they are attached to form an optionally substituted C₆₋₁₄aryl or C₅₋₁₄heteroaryl moiety; and
Z⁵ is CH₂, NH or O.

In another particular embodiment of the compounds of formula (I), the compound is one wherein:
A is N; m is 1 or 2; n is 1 or 2; p is 0 or 1;
R² and R²′ are each independently selected from H and C₁₋₁₀alkyl; and
R³ and R³′ are each independently selected from H and C₁₋₁₀alkyl.

In another particular embodiment of the compounds of formula (I), the compound is one wherein:
A is N; B is CH; m is 1 or 2; n is 1 or 2; p is 0 or 1;
R² and R²′ are each independently selected from H and C₁₋₁₀alkyl; and
R³and R³′ are each independently selected from H and C₁₋₁₀alkyl.

In this embodiment, when the compound of formula (I) is a compound of formula (Ia) or (Ib), X may in particular be O or S. Alternatively, X may be CH, NH, NC₁₋₁₀alkyl or NC(O)C₁₋₁₀alkyl.

In a further embodiment of the compounds of formula (I), the compound is one wherein:
A is N; B is N; m is 1 or 2; n is 1 or 2; p is 0 or 1;
R² and R²′ are each independently selected from H and C₁₋₁₀alkyl; and
R³ and R³′ are each independently selected from H and C₁₋₁₀alkyl.

In this embodiment, when the compound of formula (I) is a compound of formula (Ia) or (Ib), X may in particular be O or S. Alternatively, X may be C11, NH, NC₁₋₁₀alkyl or NC(O)C₁₋₁₀alkyl.

In a particular embodiment of the compounds of formula (I), the compound is one wherein:
A is N; m is 1 or 2; n is 1 or 2; p is 0 or 1;
R² and R²′ are each independently selected from H and C₁₋₁₀alkyl;
R³ and R³′ are each independently selected from H and C₁₋₁₀alkyl; and
R⁵ H, Br, Cl, F, NH₂, NO₂, CF₃, CN, methyl, methoxy, NHMe, acetyl, acetate or acetamido.

In a further embodiment of the compounds of formula (I), the compound is one wherein:
A is N; m is 1 or 2; n is 1 or 2; p is 0 or 1;
R² and R²′ are each independently selected from H and C₁₋₁₀alkyl;
R³ and R³′ are each independently selected from H and C₁₋₁₀alkyl; and
Y is selected from:

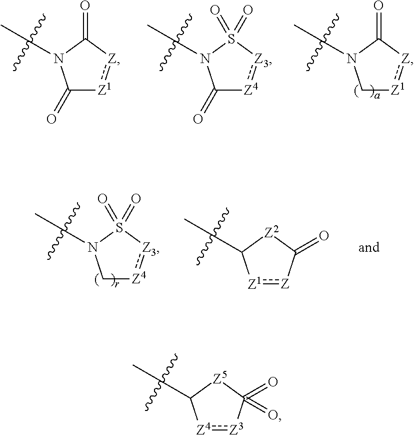

herein a, r, Z, Z₁, Z², Z³, Z⁴ and Z⁵ are as defined above.

In a particular embodiment of the compounds of formula (I):
A is N;
R¹ is H, C₁₋₁₀alkyl or C₃₋₄₀cycloalkyl;
R² and R²′ are each independently selected from H, C₁₋₁₀alkyl and C₃₋₁₀cycloalkyl;
R³ and R³′ are each independently selected from H, C₁₋₁₀alkyl and C₃₋₁₀cycloalkyl;
R⁴ is H, F, Cl, Br, I, NH₂, N(R^{m})₂, CF₃, NO₂, CN, C₁₋₁₀alkyl, C₁₋₁₀alkoxy, C₁₋₁₀alkylamino, C_{6 -14}aryl, C₅₋₁₄heteroaryl, —OC(O)Rⁿ, C(O)Rⁿ or NHC(O)Rⁿ; wherein each R^{m} is independently selected from C₁₋₁₀alkyl (particularly C₁₋₄alkyl) and C(O)Rⁿ, wherein Rⁿ is C₁₋₄alkyl, C₁₋₄alkoxy or C₁₋₄alkylamino;
R⁵ is F, Cl, Br, I, NH₂, N(R^{s})₂, CF₃, NO₂, CN, C₁₋₁₀alkyl, C₁₋₁₀alkoxy, C₁₋₁₀alkylamino, C₆₋₁₄aryl, C₅₋₁₄heteroaryl, —OC(O)R^{w}, C(O)R^{w} or NHC(O)R^{w}; wherein each R⁵ is independently selected from C₁₋₁₀alkyl (particularly C₁₋₄alkyl) and C(O)R^{w}; wherein R^{w} is C₁₋₄alkyl, C₁₋₄alkoxy or C₁₋₄alkylamino; or R⁵ is taken together with the carbon atom to which it is attached and the adjacent carbon atom to form a 5 or 6-membered ring in a compound according to formula (Ia) or (Ib), as defined above;
wherein, X is CH₂, NH, O or S;
R⁶ is H, F, Cl, Br, I, NH₂, N(R^{d})₂, CF₃, NO₂, CN, C₁₋₁₀alkyl, C₁₋₁₀alkoxy, C₁₋₁₀alkylamino, C₆₋₁₄aryl, C₅₋₁₄heteroaryl, —OC(O)R^{v}, C(O)R^{v} or NHC(O)R^{v}; wherein each R^{d} is independently selected from C₁₋₁₀alkyl (particularly C₁₋₄alkyl) and C(O)^{v}, wherein R^{v} is C₁₋₄alkyl, C₁₋₄alkyloxy or C₁-C₄alkylamino;
Y is selected from:

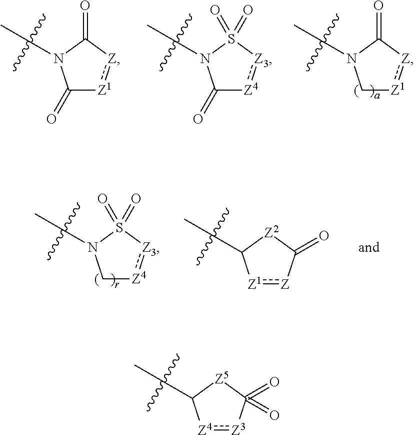

wherein a, r, Z, Z¹, Z², Z³, Z⁴ and Z⁵ are as defined above.

In this embodiment, B may in particular be CH. Alternatively, B may be N.

### Further Embodiments of the Compounds of Formula (I)

General

Various embodiments of the compounds of formula (I) are described in this application. The skilled person will recognise that features specified in each of these embodiments may be combined with other features specified in other embodiments to provide further embodiments of the invention.

A and B

In the compounds of formula (I), A and B are each independently CH or N.

Typically, A is N and B is CH. However, in some embodiments, A is N and B is N. In further embodiments, A is CH and B is N. In yet further embodiments, A is CH and B is CH.

m, n and p

In the compounds of formula (I), m, n and p are each independently 0, 1 or 2.

Typically, m is 1. However, in some embodiments, m is 2. In further embodiments, m is 0.

Typically, n is 1. However, in some embodiments, n is 2. In further embodiments, n is 0.

Typically, p is 0. However, in some embodiments, p is 1. In further embodiments, p is 2.

Typically, m+n=2. In particular, m and n are each 1. However, in some embodiments, m+n=3. In particular, m is 1 and n is 2. In further embodiments, m is 2 and n is 1. In still further embodiments,

m+n=4.

Typically, m+n+p=2. For example, m and n are each 1 and p is 0. However, in some embodiments, m+n+p=3. For example, m, n and p are each 1 or m is 1, n is 2 and p is 0. In further embodiments, m+n+p=4. For example, m is 1, n is 2 and p is 1. In yet further embodiments, m+n+p=0, 1, 5 or 6.

Group R¹

In the compounds of formula (I), R¹ is H or optionally substituted C₁₋₁₀alkyl, C₃₋₁₀cycloalkyl, C₁-C₁₁heteroalkyl, C₃₋₁₀heterocycloalkyl, C₆₋₁₄aryl or C₅₋₁₄heteroaryl.

In some embodiments, R¹ is H or optionally substituted C₁₋₁₀alkyl or C₃₋₁₀cycloalkyl. In particular, R¹ may be H. In other embodiments, R¹ is optionally substituted C₁₋₁₀alkyl, C₃₋₁₀cycloalkyl, C₁-C₁₁heteroalkyl or C₃₋₁₀heterocycloalkyl, in particular, C₁₋₁₀alkyl or C₃₋₁₀cycloalkyl. In further embodiments, R¹ is C₁-C₁₁heteroalkyl or C₃₋₁₀heterocycloalkyl. In further embodiments, R¹ is C₆₋₁₄aryl or C₅₋₁₄heteroaryl. In yet further embodiments, R¹ is H or C₁₋₁₀alkyl. In particular, R¹ is C₁₋₁₀alkyl, particularly C₁₋₄alkyl, for example, methyl. In these embodiments, R¹ may be unsubstituted.

Groups R², R²′, R³ and R³′

In the compounds of formula (I), R², R²′, R³ and R³′ are each independently H or optionally substituted C₁₋₁₀alkyl or C₃₋₁₀cycloalkyl.

Typically, R², R²′, R³ and R³′ are each independently H, C₁₋₁₀alkyl or C₃₋₁₀cycloalkyl. For example, R², R²′, R³ and R³′ may each independently be H or C₁₋₁₀alkyl, in particular H or C₁₋₆alkyl. In specific embodiments, R², R²′, R³ and R³′ are each independently H or methyl. For example, R², R²′, R³ and R³′ may all be H.

In some embodiments, R²≠R²′. Similarly, in some embodiments, R³≠R³′. In further embodiments, R²≠R²′ and R³≠R³′.

In some embodiments, R² is H and R²′ is H, C₁₋₁₀alkyl or C₃₋₁₀cycloalkyl. Similarly, in some embodiments, R³ is H and R³′ is selected from H, C₁₋₁₀alkyl and C₃₋₁₀cycloalkyl. In particular embodiments, R² and R³ are each H and R²′ and R³′ are each independently selected from H and C₁₋₆alkyl. In further embodiments, each of R² and R³ is H while each of R²′ and R³′ is C₁₋₆alkyl, particularly methyl.

Group R⁴

In the compounds of formula (I), R⁴ is H, NH₂, NO₂, halo, CN or optionally substituted C₁₋₁₀alkyl, C₁₋₁₁heteroalkyl, C₆₋₁₄aryl or C₅₋₁₄heteroaryl.

In particular embodiments of the compounds of formula (I), R⁴ is H, halo, CN or optionally substituted C₁₋₁₀alkyl, C₁₋₁₁heteroalkyl, C₆₋₁₄aryl or C₅₋₁₄heteroaryl .

When R⁴ is optionally substituted C₁₋₁₀alkyl it may, in particular, be optionally substituted C₁-C₄alkyl, particularly optionally substituted methyl. In some embodiments, the optionally substituted methyl is —C(O)Rⁿ, wherein Rⁿ is C₁₋₆alkyl, C₁₋₆alkoxy or C₁₋₆alkylamino. In particular, Rⁿ may be methyl, methoxy or methylamino. For example, R⁴ is acetyl.

When R⁴ is optionally substituted C₁₋₁₁heteroalkyl, it may, in particular, be NH₂, N(R^{m})₂, NO₂ or optionally substituted C₁₋₁₀alkoxy or C₁₋₁₀alkylamino, wherein each R^{m} is independently selected from C₁₋₁₀alkyl and —C(O)Rⁿ, wherein Rⁿ is as defined above.

In some embodiments, when R⁴ is optionally substituted C₁₋₁₁heteroalkyl, it may in particular be optionally substituted C₁₋₁₀alkoxy, particularly optionally substituted C₁-C₄alkoxy. For example, it may be optionally substituted methoxy. In some embodiments, the optionally substituted methoxy is OC(O)Rⁿ, wherein Rⁿ is as defined above. For example, R⁴ is acetate.

In some embodiments, when R⁴ is optionally substituted C₁₋₁₁heteroalkyl, it may, in particular, be optionally substituted C₁₋₁₀alkylamino, particularly, optionally substituted C₁-C₄alkylamino. For example, it may be optionally substituted methylamino. In some embodiments, the optionally substituted methylamino is —NHC(O)Rⁿ, wherein Rⁿ is as defined above. For example, R⁴ is acetamido.

In some embodiments, when R⁴ is optionally substituted C₁₋₁₁heteroalkyl, it may, in particular, be NH₂, NH(R^{m}), N(R^{m})₂ or NO₂, wherein each R^{m} is independently selected from C₁₋₁₀alkyl and —C(O)Rⁿ, wherein Rⁿ is as defined above. In these embodiments, R^{m} may in particular be independently selected from C₁₋₄alkyl and C(O)Rⁿ; wherein Rⁿ is C₁₋₄alkyl, C₁₋₄alkoxy or C₁₋₄alkylamino. In particular, Rⁿ may be methyl, methoxy or methylamino.

In further embodiments, R⁴ is H, F, Cl, Br, I, NH₂, N(R^{m})₂, CF₃, NO₂, CN, C₁₋₁₀alkyl, C₁₋₁₀alkoxy, C₁₋₁₀alkylamino, C₆₋₁₄aryl, C₅₋₁₄heteroaryl, —OC(O)Rⁿ, C(O)Rⁿ or NHC(O)Rⁿ; wherein each R^{m} is independently selected from C₁₋₁₀alkyl (particularly C₁₋₄alkyl) and C(O)Rⁿ; wherein Rⁿ is C₁₋₄alkyl, C₁₋₄alkoxy or C₁₋₄alkylamino. In particular, Rⁿ may be methyl, methoxy or methylamino. In particular, R⁴ is H, Br, Cl, F, NH₂, CF₃, NO₂, CN, methyl, methoxy, methylamino, acetyl, acetate or acetamido.

In other embodiments R⁴ is C₆₋₁₄aryl or C₅₋₁₄heteroaryl, for example, phenyl or pyridine. Typically, R⁴ is H.

Group R⁵

In the compounds of formula (I), R⁵ is H, NH₂, NO₂, halo, CN or optionally substituted C₁₋₁₀alkyl, C₁₋₁₁heteroalkyl, C₆₋₁₄aryl or C₅₋₁₄heteroaryl; or R⁵ is taken together with the carbon atom to which it is attached and the adjacent carbon atom to form a 5 or 6 membered ring in a compound of formula (Ia) or (Ib) as defined above, wherein,
X is CH₂, NH, NC₁₋₁₀alkyl, NC(O)C₁₋₁₀ alkyl, O or S;
R⁶ is H, NH₂, NO₂, halo, CN or optionally substituted C₁₋₁₀alkyl, C₁₋₁₁heteroalkyl, C₆₋₁₄aryl or C₅₋₁₄heteroaryl; and
q is 1 or 2.

In particular embodiments of the compounds of formula (I), R⁵ is H, halo, CN or optionally substituted C₁₋₁₀alkyl, C₁₋₁₁heteroalkyl, C₆₋₁₄aryl or C₅₋₁₄heteroaryl; or R⁵ is taken together with the carbon atom to which it is attached and the adjacent carbon atom to form a 5 or 6 membered ring in a compound of formula (Ia) or (Ib) as defined above, wherein,
X is CH₂, NH, NC₁₋₁₀alkyl, NC(O)C₁₋₁₀ alkyl, O or S;
R⁶ is H, halo, CN or optionally substituted C₁₋₁₀alkyl, C₁₋₁₁heteroalkyl, C₆₋₁₄aryl or C₅₋₁₄heteroaryl; and
q is 1 or 2.

In some embodiments, R⁵ is H, NH₂, NO₂, halo, CN or optionally substituted C₁₋₁₀alkyl, C₁₋₁₁heteroalkyl, C₆₋₁₄aryl or C₅₋₁₄heteroaryl.

In some embodiments, R⁵ is H, halo, CN or optionally substituted C₁₋₁₀alkyl, C₁₋₁₁heteroalkyl, C₆₋₁₄aryl or C₅₋₁₄heteroaryl.

When R⁵ is optionally substituted C₁₋₁₀alkyl it may, in particular, be optionally substituted C₁-C₄alkyl, particularly optionally substituted methyl. For example, optionally substituted methyl may be —C(O)R^{w}, wherein R^{w} is C₁₋₆alkyl, C₁₋₆alkoxy or C₁₋₆alkylamino. In particular, R^{w} may be methyl, methoxy or methylamino. For example, R⁵ is acetyl.

When R⁵ is optionally substituted C₁₋₁₁heteroalkyl, it may, in particular, be NH₂, N(R^{s})₂, NO₂ or optionally substituted C₁₋₁₀alkoxy or C₁₋₁₀alkylamino, wherein each R⁵ is independently selected from C₁₋₁₀alkyl and —C(O)R^{w}, wherein R^{w} is as defined above.

In some embodiments, when R⁵ is optionally substituted C₁₋₁₁heteroalkyl, it may, in particular, be optionally substituted C₁₋₁₀alkoxy, particularly C₁-C₄alkoxy. For example, it may be optionally substituted methoxy. In some embodiments, the optionally substituted methoxy is —OC(O)R^{w}, wherein R^{w} is as defined above. For example, R⁵ is acetate.

In some embodiments, when R⁵ is optionally substituted C₁₋₁₁heteroalkyl, it may, in particular, be optionally substituted C₁₋₁₀alkylamino, particularly, optionally substituted C₁-C₄alkylamino. For example, it may be optionally substituted methylamino. In some embodiments, the optionally substituted methylamino is —NHC(O)R^{w}, wherein R^{w} is as defined above. For example, R⁵ is acetamido.

In some embodiments, when R⁵ is optionally substituted. C₁₋₁₁heteroalkyl, it may, in particular, be NH₂, NH(R^{s}), N(R^{s})₂ or NO₂, wherein each R^{s} is independently selected from C₁₋₁₀alkyl and —C(O)R^{w}, wherein R^{w} is as defined above. In these embodiments, R^{s} may in particular be independently selected from C₁₋₄alkyl and C(O)R^{w}; wherein R^{w} is C₁₋₄alkyl, C₁₋₄alkoxy or C₁₋₄alkylamino. In particular, R^{w} may be methyl, methoxy or methylamino.

In further embodiments, R⁵ is H, F, Cl, Br, I, NH₂, N(R^{s})₂, CF₃, NO₂, CN, C₁₋₁₀alkyl, C₁₋₁₀alkoxy, C₁₋₁₀alkylamino, C₆₋₁₄aryl, C₅₋₁₄heteroaryl, —OC(O)R^{w}, —C(O)R^{w} or NHC(O)R^{w}; wherein each R^{s} is independently selected from C₁₋₁₀alkyl (particularly Q₁₋₄alkyl) and —C(O)R^{w}; wherein R^{w} is C₁₋₄alkyl, C₁₋₄alkoxy or C₁₋₄alkylamino. In particular, R^{w} may be methyl, methoxy or methylamino. In particular, R⁵ is H, Br, Cl, F, NH₂, NO₂, CF₃, CN, methyl, methoxy, methylamino, acetyl, acetate or acetamido.

In specific embodiments, R⁵ is methoxy.

In other particular embodiments, R⁵ is halo, for example F, Cl, Br or I. In further specific embodiments, R⁵ is F.

In other embodiments, R⁵ is C₆₋₁₄aryl or C₅₋₁₄heteroaryl, for example, phenyl or pyridine.

In yet further embodiments, R⁵ is CN.

In other embodiments, R⁵ is taken together with the carbon atom to which it is attached and the adjacent carbon atom to form a 5 or 6 membered ring in a compound of formula (Ia) or (Ib).

In some of these embodiments, the compound of formula (Ia) is, in particular, a compound of formula (IIa):

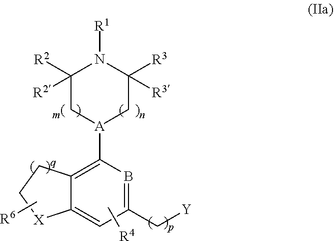

In other embodiments, the compound of formula (Ib) is, in particular, a compound of formula (IIb):

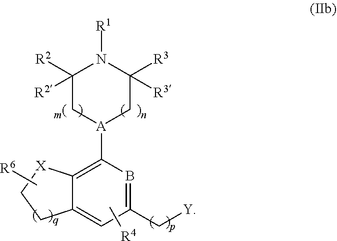

When the compound is of formula (Ia), it may, in particular, be a compound of formula (IIIa):

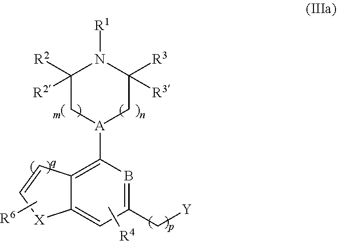

When the compound is of formula (Ib), it may, in particular, be a compound of formula (IIIb):

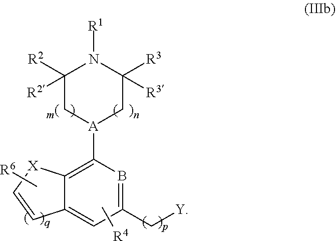

In some embodiments, X is CH₂. In other embodiments, X is NH, NC₁₋₁₀alkyl or NC(O)C₁₋₁₀alkyl, in particular, NH. In yet further embodiments, X is O or S. In particular, X is O. In other embodiments, X is S.

Typically, q is 1. In further embodiments, q is 2.

For example, when the compound is of formula (IIIb), it may, in particular, be a compound of formula (IVa) or (IVb):

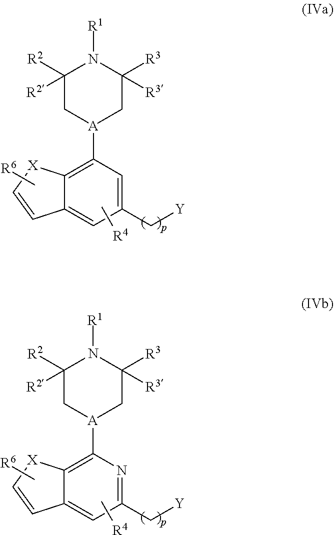

When the compound is of formula (IVa) or (IVb), it may, in particular, be a compound of formula (Va) or (Vb):

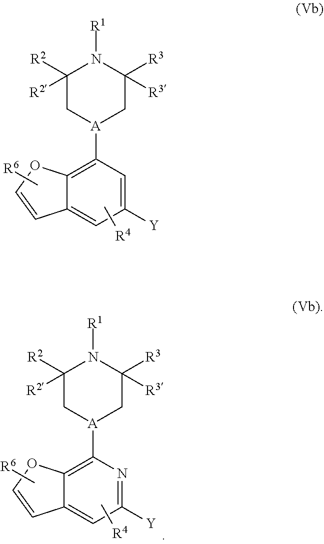

In each of these embodiments, A may be N when B is CH. In some embodiments, A is N and B is N. In further embodiments, A is CH and B is N. In yet further embodiments, A is CH and B is CH.

Group R⁶

In some embodiments R⁶ is H, NH₂, NO₂, halo, CN or optionally substituted C₁₋₁₀alkyl, C₁₋₁₁heteroalkyl, C₆₋₁₄aryl or C₅₋₁₄heteroaryl.

In some embodiments R⁶ is H, halo, CN or optionally substituted C₁₋₁₀alkyl, C₁₋₁₁heteroalkyl, C₆₋₁₄aryl or C₅₋₁₄heteroaryl.

When R⁶ is optionally substituted C₁₋₁₀alkyl, it may, in particular, be optionally substituted C₁₋₄alkyl, particularly optionally substituted methyl. For example, the optionally substituted methyl may be —C(O)R^{v}, wherein R^{v} is C₁₋₆alkyl, C₁₋₆alkoxy or C₁₋₆alkylamino. In particular, R^{v} may be methyl, methoxy or methylamino. For example, R⁶ is acetyl.

When R⁶ is optionally substituted C₁₋₁₁heteroalkyl, it may, in particular, be NH₂, N(R^{d})₂, NO₂ or optionally substituted C₁₋₁₀alkoxy or C₁₋₁₀alkylamino, wherein each R^{d} is independently selected from C₁₋₁₀alkyl and —C(O)R^{v}, wherein R^{v} is as defined above.

In some embodiments, the optionally substituted C₁₋₁₁heteroalkyl, may, in particular, be optionally substituted C₁₋₁₀alkoxy, particularly optionally substituted C₁₋₄alkoxy. For example, it may be optionally substituted methoxy. In some embodiments, the optionally substituted methoxy is —OC(O)R^{v}, wherein R^{v} is as defined above. For example, R⁶ is acetate.

In some embodiments, the optionally substituted C₁₋₁₁heteroalkyl may, in particular, be optionally substituted C₁₋₁₀alkylamino, particularly optionally substituted C₁₋₄alkylamino. For example, it may be optionally substituted methylamino. In some embodiments, the optionally substituted methylamino is —NHC(O)R^{v} wherein R^{v} is as defined above. For example, R⁶ is acetamido.

In some embodiments, when R⁶ is optionally substituted C₁₋₁₁heteroalkyl, it may, in particular, be NH₂, NH(R^{d}), N(R^{d})₂ or NO₂, wherein each R^{d} is independently selected from C₁₋₁₀alkyl and —C(O)R^{v}, wherein R^{v} is as defined above. In these embodiments, R^{d} may in particular be independently selected from C₁₋₄alkyl and C(O)R^{v}; wherein R^{v} is C₁₋₄alkyl, C₁₋₄alkoxy or C₁₋₄alkylamino.

In further embodiments, R⁶ is H, F, Cl, Br, I, NH₂, N(R^{d})₂, CF₃, NO₂, CN, C₁₋₁₀alkyl, C₁₋₁₀alkoxy, C₁₋₁₀alkylamino, C₆₋₁₄aryl, C₅₋₁₄heteroaryl, —OC(O)R^{v}, C(O)R^{v} or NHC(O)R^{v}; wherein each R^{d} is independently selected from C₁₋₁₀alkyl (particularly C₁₋₄alkyl) and C(O)R^{v}; wherein R^{v} is C₁₋₄alkyl, C₁₋₄alkoxy or C₁₋₄alkylamino. In particular, R⁶ is H, Br, Cl, F, NH₂, NO₂, CF₃, CN, methyl, methoxy, methylamino, acetyl, acetate or acetamido.

In other embodiments R⁶ is C₆₋₁₄aryl or C₅₋₁₄heteroaryl, for example, phenyl or pyridine.

In some embodiments, R⁶ may be H, halo, NH₂, CF₃, C₁₋₁₀alkyl or C₁₋₁₀alkoxy. In particular, R⁶ may be H, halo, NH₂, CF₃, methoxy or methyl, particularly H.

Group Y

In the compounds of formula (I), Y is optionally substituted C₃₋₁₀heterocycloalkyl, C₅₋₁₀heterocycloalkenyl or C₅₋₁₄heteroaryl.

In particular, Y may be optionally substituted C₅₋₆heterocycloalkyl, C₅₋₆heterocycloalkenyl or C₅₋₆heteroaryl. In further embodiments, Y is C₃₋₁₀heterocycloalkyl or C₅₋₁₄heterocycloalkenyl, for example C₃₋₁₀heterocycloalkyl. In other embodiments, Y is C₅₋₁₄heterocycloalkenyl. In yet further embodiments, Y is C₅₋₁₄heteroaryl. Typically, at least one optional substituent is ═O. In some embodiments, Y is unsubstituted.

In some embodiments, Y is C₃₋₁₀heterocycloalkyl, C₅₋₁₀heterocycloalkenyl or C₅₋₁₄heteroaryl each optionally substituted with one or more substituents independently selected from the group consisting of halogen, trihalomethyl, trihaloethyl, —NO₂, —CN, —N⁺(C₁₋₆alkyl)₂O⁻, —CO₂H, —CO₂C₁₋₆alkyl, —SO₃H, —SOC₁₋₆alkyl, —SO₂C₁₋₆alkyl, —SO₃C₁₋₆alkyl, —OC(═O)OC₁₋₆alkyl, —C(═O)H, —C═O)C₁₋₆alkyl, —OC(═O)C₁₋₆alkyl, ═O, —N(C₁₋₆alkyl)₂, —C(═O)NH₂, —C(═O)N(C₁₋₆alkyl)₂, —N(C₁₋₆alkyl)C(═O)O(C₁₋₆alkyl), —N(C₁₋₆alkyl)C(═O)N(C₁₋₆alkyl)₂, —OC(═O)N(C₁₋₆alkyl)₂, —N(C₁₋₆alkyl)C(═O)C₁₋₆alkyl, —C(═S)N(C₁₋₆alkyl)₂, —N(C₁₋₆alkyl)C(═S)C₁₋₆alkyl, —SO₂N(C₁₋₆alkyl)₂, —N(C ₁₋₆alkyl)SO₂C₁₋₆alkyl, —N(C₁₋₆alkyl)C(═S)N(C₁₋₆alkyl)₂, —N(C₁₋₆alkyl)SO₂N(C₁₋₆alkyl)₂, and optionally substituted C₁₋₁₀alkyl, C₁₋₁₁heteroalkyl, C₃₋₁₀cycloalkyl, C₃₋₁₀heterocycloalkyl, C₂₋₆alkenyl, C₂₋₆heteroalkenyl, C₃₋₆cycloalkenyl, C₅₋₁₀heterocycloalkenyl, C₂₋₆alkynyl, C₂₋₆heteroalkynyl, C₆₋₁₄aryl, C₅₋₁₄-heteroaryl, —Z^{u}—C₁₋₆alkyl, —Z^{u}—C₃₋₆cycloalkyl, —Z^{u}—C₂₋₆alkenyl, —Z^{u}—C₃₋₆cycloalkenyl and —Z^{u}—C₂₋₆alkynyl; wherein two adjacent substituents taken together with the C or N atoms of the Y group to which they are attached may form an optionally substituted C₆₋₁₄aryl or C₅₋₁₄heteroaryl moiety; and

wherein
Z^{u} is independently O, S, NH or N(C₁₋₆alkyl).

In a particular embodiment, the one or more optional Y group substituents may be independently selected from the group consisting of halogen, trihalomethyl, trihaloethyl, —NO₂, —CN, —N⁺(C₁₋₆alkyl)₂O, —CO₂H, —CO₂C₁₋₆alkyl, —SO₃H, —SOC₁₋₆alkyl, —SO₂C₁₋₆alkyl, —SO₃C₁₋₆alkyl, —OC(═O)OC₁₋₆alkyl, —C(═O)H, —C(═O)C₁₋₆alkyl, —OC(═O)C₁₋₆alkyl, ═O, —N(C₁₋₆alkyl)₂, —C(═O)NH₂, —C(═O)N(C₁₋₆alkyl)₂, —N(C₁₋₆alkyl)C(═O)O(C₁₋₆alkyl), —N(C₁₋₆alkyl)C(═O)N(C₁₋₆alkyl)₂, —OC(═O)N(C₁₋₆alkyl)₂, —N(C₁₋₆alkyl)C(═O)C₁₋₆alkyl, —C(═S)N(C₁₋₆alkyl)₂, —N(C₁₋₆alkyl)C(═S)C₁₋₆alkyl, —SO₂N(C₁₋₆alkyl)₂, —N(C₁₋₆alkyl)SO₂C₁₋₆alkyl, —N(C₁₋₆alkyl)C(═S)N(C₁₋₆alkyl)₂, —N(C₁₋₆alkyl)SO₂N(C₁₋₆alkyl)₂, and optionally substituted C₁₋₆alkyl, C₁₋₆heteroalkyl, C₃₋₆cycloalkyl, C₃₋₆heterocycloalkyl, C₂₋₆alkenyl, C₂₋₆heteroalkenyl, C₃₋₆cycloalkenyl, C₅₋₁₀heterocycloalkenyl, C₂₋₆alkynyl, C₂₋₆heteroalkynyl, C₆₋₁₄aryl, C₅₋₁₄heteroaryl, —Z^{u}—C₁₋₆alkyl, —Z^{u}—C₃₋₆cycloalkyl, —Z^{u}—C₂₋₆alkenyl, —Z^{u}—C₃₋₆cycloalkenyl and —Z^{u}13 C₂₋₆alkynyl; wherein two adjacent substituents taken together with the C or N atoms of the Y group to which they are attached may form an optionally substituted C₆₋₁₄aryl or C₅₋₁₄heteroaryl moiety; and

wherein
Z^{u} is independently O, S, NH or N(C₁₋₆alkyl).

In other particular embodiments, the one or more optional Y group substituents may be independently selected from the group consisting of ═O and optionally substituted C₁₋₁₀alkyl, C₁₋₁₁heteroalkyl, C₃₋₁₀cycloalkyl, C₃₋₁₀heterocycloalkyl, C₅₋₁₀heterocycloalkenyl, C₆₋₁₄aryl and C₅₋₁₄heteroaryl. For example, one or more optional substituents on Y may be selected from ═O and optionally substituted C₁₋₁₀alkyl and C₆₋₁₄aryl (such as optionally substituted phenyl).

In some embodiments, the one or more optional Y group substituents may be independently selected from the group consisting of ═O and optionally substituted C₁₋₆alkyl, C₁₋₆heteroalkyl, C₃₋₆cycloalkyl, C₃₋₆heterocycloalkyl, C₅₋₁₀heterocycloalkenyl, C₆₋₁₄aryl and C₅₋₁₄heteroaryl. For example, one or more optional substituents on Y may be selected from ═O and optionally substituted C₁₋₆alkyl and C₆₋₁₄aryl (such as optionally substituted phenyl).

In particular embodiments, the one or more optional Y group substituents may be independently selected from the group consisting of ═O, C₁₋₆alkyl, C₁₋₆heteroalkyl, C₃₋₆cycloalkyl, C₃₋₆heterocycloalkyl, C₅₋₁₀heterocycloalkenyl, C₆₋₁₄aryl and C₅₋₁₄heteroaryl. For example, one or more optional substituents on Y may be selected from ═O and optionally substituted C₁₋₆alkyl and C₆₋₁₄aryl (such as optionally substituted phenyl). In particular embodiments, the optionally substituted Y group may be C₅₋₆heterocycloalkyl, C₅₋₆heterocycloalkenyl or C₅₋₆heteroaryl. In further embodiments, the optionally substituted Y group is C₃₋₁₀heterocycloalkyl or C₅₋₁₄heterocycloalkenyl, for example C₃₋₁₀heterocycloalkyl. In other embodiments, the optionally substituted Y group is C₅₋₁₄heterocycloalkenyl. In yet further embodiments, the optionally substituted Y group is C₅₋₁₄heteroaryl. Typically, at least one optional substituent is ═O. In some embodiments, Y is unsubstituted.

In some embodiments, Y is selected from:

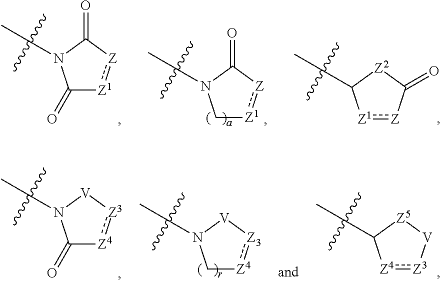

wherein
   each of a and r is independently 0, 1, 2 or 3;
   Z is CR⁷ or C(R⁷)₂ and Z¹ is CR⁸ or C(R⁸)₂, or
   Z is CR⁷ or C(R⁷)₂ and Z¹ is N, NR⁸, O or S, or
   Z is N, NR⁷, O or S and Z¹ is CR⁸ or C(R⁸)₂, wherein
      each R⁷ and R⁸ is independently selected from H and optionally substituted C₁₋₁₀alkyl, C₁₋₁₁heteroalkyl, C₃₋₁₀cycloalkyl, C₃₋₁₀heterocycloalkyl, C₅₋₁₀heterocycloalkenyl, C₆₋₁₄aryl and C₅₋₁₄heteroaryl; or R⁷ and R⁸ are taken together with the C or N atoms to which they are attached to form an optionally substituted C₆₋₁₄aryl or C₅₋₁₄heteroaryl moiety;
   Z² is CH₂, NH, O or S;
   V is S(O)_{y}, wherein
      y is 1 or 2;
   Z³ is CR⁹ or C(R⁹)₂ and Z⁴ is CR¹⁰ or C(R¹⁰)₂, or
   Z³ is CR⁹ or C(R⁹)₂ and Z⁴ is N, NR¹⁰ or 0, or
   Z³ is N, NR⁹ or O and Z⁴ is CR¹⁰ or C(R¹⁰)₂, wherein
      each R⁹ and R¹⁰ is independently selected from H and optionally substituted C₁₋₁₀alkyl, C₁₋₁₁heteroalkyl, C₃₋₁₀cycloalkyl, C₃₋₁₀heterocycloalkyl, C₅₋₁₀heterocycloalkenyl, C₆₋₁₄aryl and C₅₋₁₄heteroaryl; or R⁹ and R¹⁰ are taken together with the C or N atoms to which they are attached to form an optionally substituted C₆₋₁₄aryl or C₅₋₁₄heteroaryl moiety; and
   Z⁵ is CH₂, NH or O.

In some embodiments, where Y is substituted with a group that is itself optionally substituted, the optional substitution may be by one or more substituents independently selected from the group consisting of halogen, trihalomethyl, trihaloethyl, OH, —NO₂, —CN, —N⁺(C₁₋₆alkyl)₂O⁻, —CO₂H, —CO₂C₁₋₆alkyl, —SO₃H, —SOC₁₋₆alkyl, —SO₂C₁₋₆alkyl, —SO₃C₁₋₆alkyl, —OC(═O)OC₁₋₆alkyl, —C(═O)H, —C(═O)C₁₋₆alkyl, —OC(═O)C₁₋₆alkyl, —OSO₂C₁₋₆alkyl, —OSO₂C₆₁₋₁₄aryl, ═O, —N(C₁₋₆alkyl)₂, —C(═O)NH₂, —C(═O)NHC₁₋₆alkyl, —C(═)N(C₁₋₆alkyl)₂, —N(C₁₋₆alkyl)C(═O)O(C₁₋₆alkyl), —N(C₁₋₆alkyl)C(═O)N(C₁₋₆alkyl)₂, —OC(═O)N(C₁₋₆alkyl)₂, —N(C₁₋₆alkyl)C(═O)C₁₋₆alkyl, —C(═S)N(C₁₋₆alkyl)₂, —N(C₁₋₆alkyl)C(═S)C₁₋₆alkyl, —SO₂NH₂, —SO₂NHC₁₋₆alkyl, —SO₂N(C₁₋₆alkyl)₂, —SO₂NHC₆₋₁₄aryl, —NHC(═O)C₁₋₆alkyl, —N(C₁₋₆alkyl)SO₂C₁₋₆alkyl, —N(C₁₋₆alkyl)C(═S)N(C₁₋₆alkyl)₂, —N(C₁₋₆alkyl)SO₂N(C₁₋₆alkyl)₂, C₁₋₁₀alkyl, C₁₋₁₁heteroalkyl, C₃₋₁₀cycloalkyl, C₃₋₁₀heterocycloalkyl, C₂₋₆alkenyl, C₂₋₆heteroalkenyl, C₃₋₆cycloalkenyl, C₅₋₁₀heterocycloalkenyl, C₂₋₆alkynyl, C₂₋₆heteroalkynyl, C₆₋₁₄aryl, C₅₋₁₄heteroaryl, —Z^{u}—C₁₋₆alkyl, —Z^{u}—C₃₋₆cycloalkyl, —Z^{u}—C₂₋₆alkenyl, —Z^{u}—C₃₋₆cycloalkenyl and —Z^{u}—C₂₋₆alkynyl; wherein
Z^{u} is independently O, S, NH or N(C₁₋₆alkyl).

In certain embodiments, where Y is substituted with a group that is itself optionally substituted, the optional substitution may be by one or more substituents independently selected from the group consisting of halogen, trihalomethyl, trihaloethyl, OH, —CN, —CO₂H, —CO₂C₁₋₆alkyl, —SO₃H, —SOC₁₋₆alkyl, —SO₂C₁₋₆alkyl, —SO₃C₁₋₆alkyl, —OC(═O)OC₁₋₆alkyl, —C(═O)H, —C(═O)C₁₋₆alkyl, —OC(═O)C₁₋₆alkyl, —OSO₂C₁₋₆alkyl, —OSO₂C₆₋₁₄aryl, ═O, —C(═O)NH₂, —C(═O)NHC₁₋₆alkyl, —C(═O)N(C₁₋₆alkyl)₂; —OC(═O)N(C₁₋₆alkyl)₂, —N(C₁₋₆alkyl)C(═O)C₁₋₆alkyl, —SO₂NH₂, —SO₂NHC₁₋₆alkyl, —SO₂N(C₁₋₆alkyl)₂, —SO₂NHC₆₋₁₄aryl, C₁₋₁₀alkyl and —Z^{u}—C₁₋₆alkyl; wherein
Z^{u} is independently O, S, NH or N(C₁₋₆alkyl).

In other embodiments, where Y is substituted with a group that is itself optionally substituted, the optional substitution may be by one or more substituents independently selected from the group consisting of halogen, CF₃, methoxy, methyl, OH, —CO₂H, —SO₂C₁₋₆alkyl, —C(═O)H, —OSO₂C₁₋₆alkyl, —OSO₂C₆₋₁₄aryl, —O, —C(═O)NHMe, —NHC(═O)Me, —SO₂NH₂, —SO₂NHC₁₋₆alkyl, —SO₂N(C₁₋₆alkyl)₂ and —SO₂NHC₆₋₁₄aryl.

Where present, a is 0, 1, 2 or 3. In some embodiments, a is 1 or 2. Typically, a is 1. In other embodiments, a is 0. In further embodiments, a is 3. Similarly, where present, r is 0, 1, 2 or 3. In some embodiments, r is 1 or 2. Typically, r is 1. In other embodiments, r is 0. In further embodiments, r is 3.

Where present, Z is CR⁷ or C(R⁷)₂ and Z¹ is CR⁸ or C(R⁸)2, or Z is CR⁷ or C(R⁷)₂ and Z¹ is N, NR⁸, O or S, or Z is N, NR⁷, O or S and Z¹ is CR⁸ or C(R⁸)₂. Typically, Z is CR⁷ or C(R⁷)₂ and Z¹ is CR⁸ or C(R⁸)₂, or Z is N, NR⁷, O or S and Z¹ is CR⁸ or C(R⁸)₂. In some embodiments, Z is CR⁷ or C(R⁷)₂ and Z¹ is N, NR⁸, O or S. In particular embodiments, when Z¹ is CR⁸ or C(R⁸)₂, Z is CR⁷ or C(R⁷)₂. In other embodiments, when Z is CR⁷ or C(R⁷)₂, Z¹ is N or NR⁸. In further embodiments, when Z is CR⁷ or C(R⁷)₂, Z¹ is O or S, particularly 0. In other embodiments, when Z¹ is CR⁸ or C(R⁸)₂, Z is N or NR⁷. In further embodiments, when Z¹ is CR⁸ or C(R⁸)₂, Z is O or S, particularly O.

Where present, Z² may be CH₂, NH, O or S. In some embodiments, Z² is CH₂, NH or O, for example CH₂. In further embodiments, Z² is O or S, particularly O. Typically, Z² is NH.

Where present, Z³ is CR⁹ or C(R⁹)₂ and Z⁴ is CR¹⁰ or C(R¹⁰)₂, or Z³ is CR⁹ or C(R⁹)₂ and Z⁴ is N, NR¹⁰, O or S, or Z is N, NR⁹, O or S and Z⁴ is CR¹⁰ or C(R¹⁰)₂. Typically, Z³ is CR⁹ or C(R⁹)₂ and Z⁴ is CR¹⁰ or)C(R¹⁰)₂, or Z³ is N, NR⁹, O or S and Z⁴ is CR¹⁰ or C(R¹⁰)₂. In some embodiments, Z³ is CR⁹ or C(R⁹)₂ and Z⁴ is N, NR¹⁰, O or S. In particular embodiments, when Z⁴ is CR¹⁰ or C(R¹⁰)₂, Z³ is CR⁹ or C(R⁹)₂. In other embodiments, when Z³ is CR⁹ or C(R⁹)₂, Z⁴ is N or NR¹⁰. In further embodiments, when Z³ is CR⁹ or C(R⁹)₂, Z⁴ is O or S, particularly O. In other embodiments, when Z⁴ is CR¹⁰ or C(R¹⁰)₂, Z³ is N or NR⁹. In further embodiments, when Z⁴ is CR¹⁰ or C(R¹⁰)₂, Z³ is O or S, particularly O.

Where present, Z⁵ may be CH₂, NH, O or S. In some embodiments, Z⁵ is CH₂, NH or O, for example CH₂. In further embodiments, Z⁵ is O or S, particularly O. Typically, Z⁵ is NH.

Where present, the bond joining Z to Z¹ and Z³ to Z⁴ may be a double or single bond. Typically, the bond is a single bond. In other embodiments, it is a double bond.

Where present, V is S(O)_{y}, wherein y is 1 or 2. Typically, y is 2. In further embodiments, y may be 1.

Where present, each R⁷ and each R⁸ is independently selected from H and optionally substituted C₁₋₁₀alkyl, C₁₋₁₁heteroalkyl, C₃₋₁₀cycloalkyl, C₃₋₁₀heterocycloalkyl, C₅₋₁₀heterocycloalkenyl, C₆₋₁₄aryl and C₅₋₁₄heteroaryl; or R⁷ and R⁸ are taken together with the C or N atoms to which they are attached to form an optionally substituted C₆₋₁₄aryl or C₅₋₁₄heteroaryl moiety. In particular embodiments, each R⁷ and each R⁸ is independently selected from H, C₁₋₁₀alkyl, C₁₋₁₁heteroalkyl, C₃₋₁₀cycloalkyl, C₃₋₁₀heterocycloalkyl, C₅₋₁₀heterocycloalkenyl, C₆₁₄aryl and C₅₋₁₄heteroaryl, particularly H. For example, each R⁷ and each R⁸ may, in particular, be independently selected from C₁₋₁₀alkyl, C₆₋₁₄aryl and C₅₋₁₄heteroaryl.

Where present, each R⁷ and each R⁸ may, in particular, be independently selected from H and optionally substituted C₁₋₁₀alkyl, C₁₋₁₁heteroalkyl, C₆₋₁₄aryl and C₅₋₁₄heteroaryl. In other embodiments, each R⁷ and each R⁸ may, in particular, be independently selected from H and optionally substituted C₁₋₁₀alkyl and C₆₋₁₄aryl, particularly optionally substituted methyl, phenyl and benzyl, for example, methoxyphenyl. The optionally substituted C₁₋₁₀alkyl may, in particular, be optionally substituted C₁₋₄alkyl, particularly optionally substituted methyl. For example, the optionally substituted methyl may be —C(O)R^{e}, wherein R^{e} is C₁₋₄alkyl, C₁₋₄alkoxy or C₁₋₄alkylamino. For example, each R⁷ and each R⁸ may be independently selected from acetyl or methyl carboxylate. In other embodiments, the optionally substituted C₁₋₁₀alkyl is lactate.

In other embodiments, each R⁷ and each R⁸ may, in particular, be independently optionally substituted C₁₋₁heteroalkyl, particularly optionally substituted C₁₋₁₀alkoxy, C₁₋₁₀alkylthio or C₁₋₁₀alkylamino, particularly, optionally substituted C₁₋₁₀alkoxy. For example, the C₁₋₁₁heteroalkyl may be optionally substituted Cₗ₋₄alkoxy, particularly optionally substituted methoxy. In some embodiments, the optionally substituted methoxy is —OC(O)R^{e} wherein R^{e} is as defined above, e.g. acetate. In another example, the optionally substituted C₁₋₁₁heteroalkyl may be optionally substituted C₁₋₁₀alkylamino, particularly optionally substituted C₁₋₄alkylamino. For example, it may be optionally substituted methylamino. In some embodiments, the optionally substituted methylamino is —NHC(O)R^{e} wherein R^{e} is as defined above, e.g. acetamido.

In some embodiments each R⁷ and each R⁸ may, in particular, be C₅₋₁₄heteroaryl. In other embodiments, each R⁷ and each R⁸ is independently selected from C₁₋₁₁heteroalkyl, C₃₋₁₀cycloalkyl, C₃₋₁₀heterocycloalkyl and C₅₋₁₀heterocycloalkenyl. In yet further embodiments, each R⁷ and each R⁸ is independently selected from H and C₁₋₁₀alkyl. For example, each R⁷ and each R⁸ is C₁₋₆alkyl. In particular embodiments, each R⁷ and each R⁸ is independently selected from H, methyl, ethyl, propyl and butyl, including tert-butyl, particularly H.

Where present, each R⁹ and each R¹⁰ is independently selected from H and optionally substituted C₁₋₁₀alkyl, C₁₋₁₁heteroalkyl, C₃₋₁₀cycloalkyl, C₃₋₁₀heterocycloalkyl, C₅₋₁₀heterocycloalkenyl, C₆₋₁₄aryl or C₅₋₁₄heteroaryl; or R⁹ and R¹⁰ are taken together with the C or N atoms to which they are attached to form an optionally substituted C₆₋₁₄aryl or C₅₋₁₄heteroaryl moiety. In particular embodiments, each of R⁹ and R¹⁰ is independently selected from H, C₁₋₁₀alkyl, C₁₋₁₁heteroalkyl, C₃₋₁₀cycloalkyl, C₃₋₁₀heterocycloalkyl, C₅₋₁₀heterocycloalkenyl, C₆₋₁₄aryl and C₅₋₁₄heteroaryl, particularly H. For example, each of R⁹ and R¹⁰ is independently selected from C₁₋₁₀alkyl, C₆₋₁₄aryl and C₅₋₁₄heteroaryl.

Where present, each R⁹ and each R¹⁰ may, in particular, be independently selected from H and optionally substituted C₁₋₁₀alkyl, C₁₋₁₁heteroalkyl, C₆₋₁₄aryl and C₅₋₁₄heteroaryl. In other embodiments, each R⁹ and each R¹⁰ may, in particular, be independently selected from H and optionally substituted C₁₋₁₀alkyl and C₆₋₁₄aryl, particularly optionally substituted methyl, phenyl and benzyl, for example, methoxyphenyl. The optionally substituted C₁₋₁₀alkyl may, in particular, be optionally substituted C₁₋₄alkyl, particularly optionally substituted methyl. For example, the optionally substituted methyl may be —C(O)R^{f}, wherein R^{f} is C₁₋₄alkyl, C₁₋₄alkoxy or C₁₋₄alkylamino. For example, each R⁹ and each R¹⁰ may be independently selected from acetyl or methyl carboxylate. In other embodiments, the optionally substituted C₁₋₁₀alkyl is lactate.

In other embodiments, each R⁹ and each R¹⁰ may, in particular, be independently optionally substituted C₁₋₁₁heteroalkyl, particularly optionally substituted C₁₋₁₀alkoxy, C₁₋₁₀alkylthio or C₁₋₁₀alkylamino, particularly, optionally substituted C₁₋₁₀alkoxy. For example, the C₁₋₁₁heteroalkyl may be optionally substituted C₁₋₄alkoxy, particularly optionally substituted methoxy. In some embodiments, the optionally substituted methoxy is —OC(O)R^{f} wherein R^{f} is as defined above, e.g. acetate. In another example, the optionally substituted C₁₋₁₁heteroalkyl may be optionally substituted C₁₋₁₀alkylamino, particularly optionally substituted C₁₋₄alkylamino. For example, it may be optionally substituted methylamino. In some embodiments, the optionally substituted methylamino is —NHC(O)R^{f} wherein R^{f} is as defined above, e.g. acetamido.

In some embodiments each R⁹ and each R¹⁰ may, in particular, be C₅₋₁₄heteroaryl. In other embodiments, each R⁹ and each R¹⁰ is independently selected from C₁₋₁₁heteroalkyl, C₃₋₁₀cycloalkyl, C₃₋₁₀heterocycloalkyl and C₅₋₁₀heterocycloalkenyl. In yet further embodiments, each R⁹ and each R¹⁰ is independently selected from H and C₅₋₁₀alkyl. For example, each R⁹ and each R¹⁰ is C₁₋₆alkyl. In particular embodiments, each R⁹ and each R^{e} is independently selected from H, methyl, ethyl, propyl and butyl, including tent-butyl, particularly H.

In some embodiments, Y is selected from:

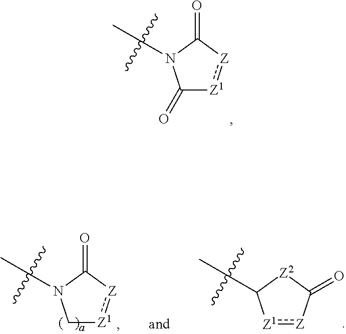

In other embodiments, Y is selected from:

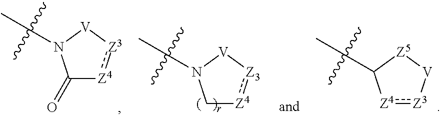

In some embodiments, Y is selected from the group consisting of:

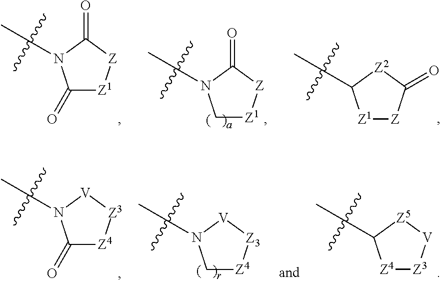

In particular embodiments, Y may be selected from the group consisting of:

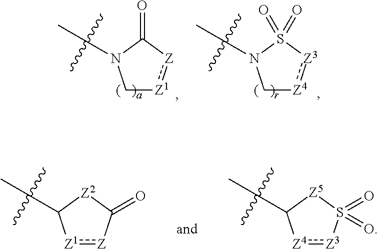

For example, Y may be

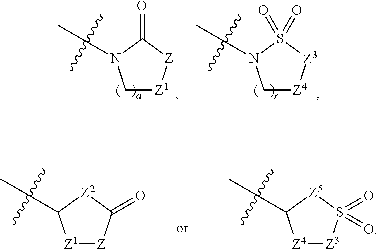

In further embodiments, Y may be selected from the group consisting of:

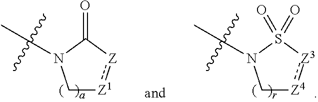

For example, Y may be

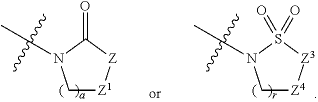

In some embodiments, Y may be selected from the group consisting of:

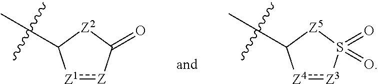

For example, Y may be

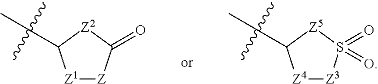

In other embodiments, Y is selected from the group consisting of:

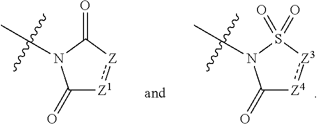

For example, Y may be

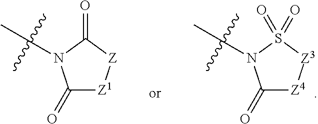

In some embodiments, Y is:

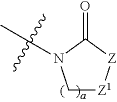

In other embodiments, Y is:

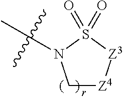

In further embodiments, Y is:

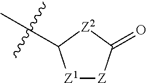

In yet further embodiments, Y is:

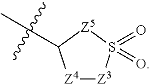

In some embodiments, Y is

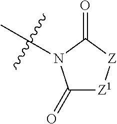

In other embodiments, Y is

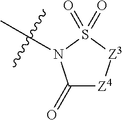

In the above embodiments,

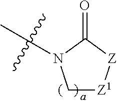

may in particular be

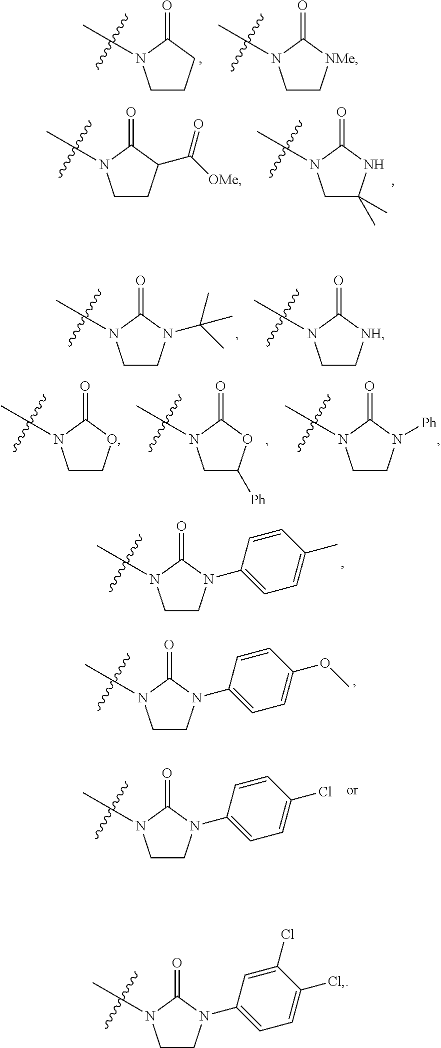

In particular embodiments,

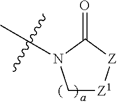

may be

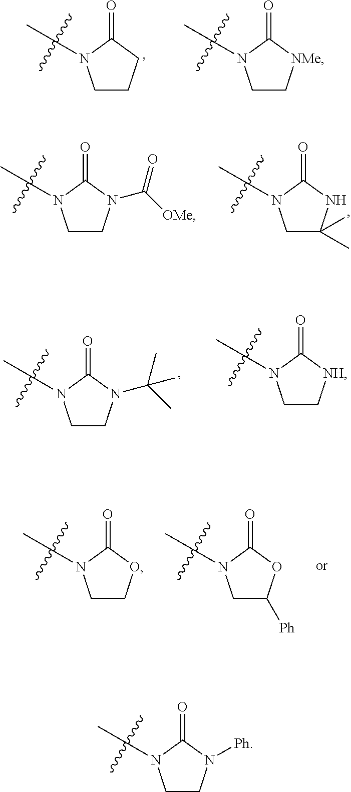

For instance, in the above embodiments

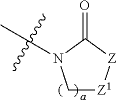

may in particular be

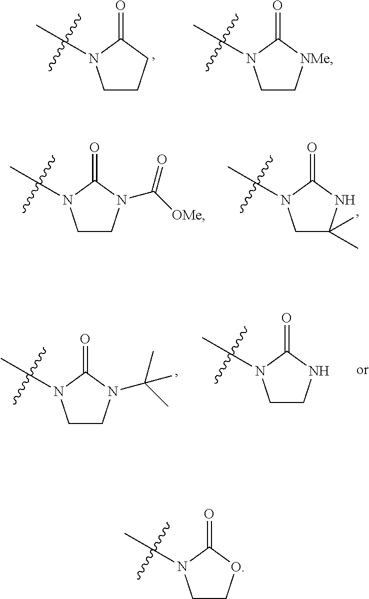

In the above embodiments,

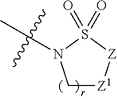

may in particular be

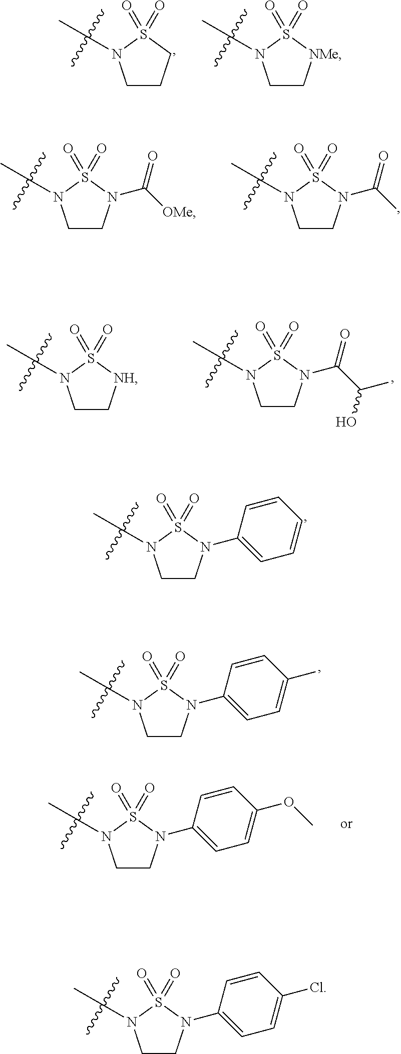

For example, in the above embodiments

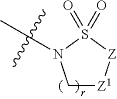

may in particular be

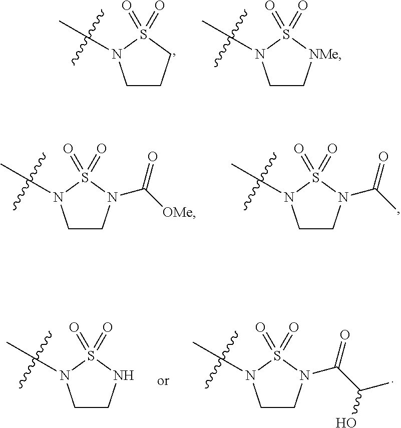

In the above embodiments,

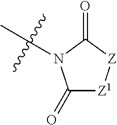

may in particular be

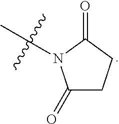 [SMILES: *N1C(=O)CCC1=O]

In the above embodiments,

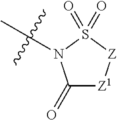

may in particular be

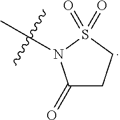 [SMILES: *N1C(=O)CCS1(=O)=O]

In the above embodiments,

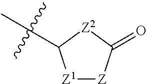

may in particular be

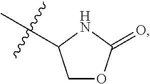 [SMILES: *C1COC(=O)N1]

for example,

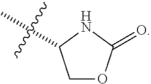 [SMILES: *[C@H]1COC(=O)N1]

For example, Y may in particular be selected from the group consisting of:

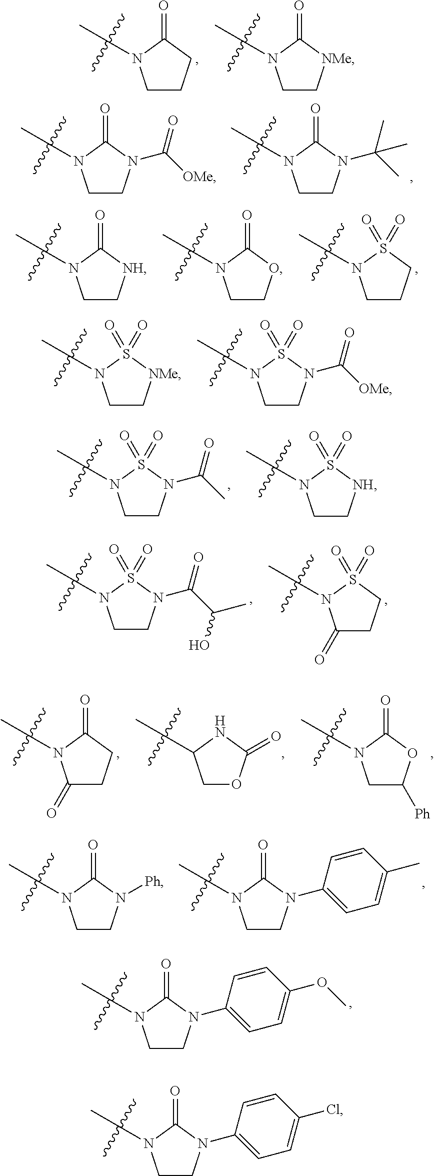

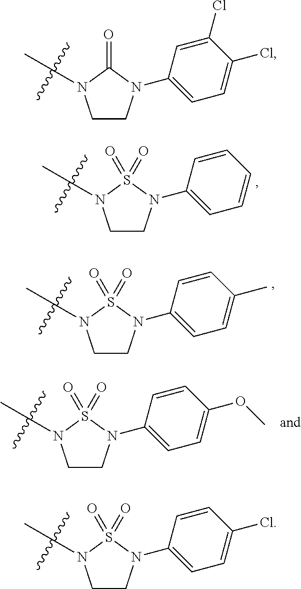

For example, Y may in particular be selected from the group consisting of:

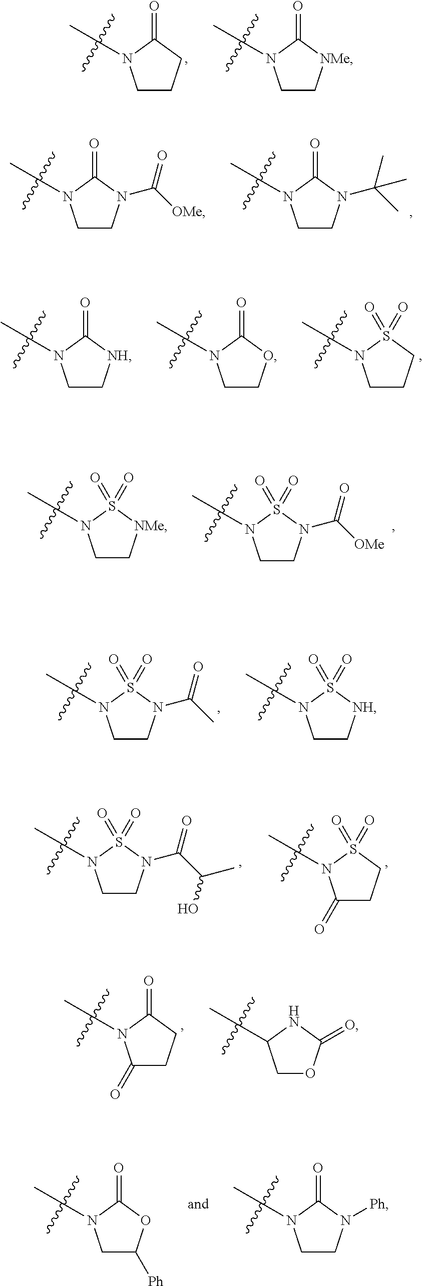

For instance, Y may in particular be selected from the group consisting of:

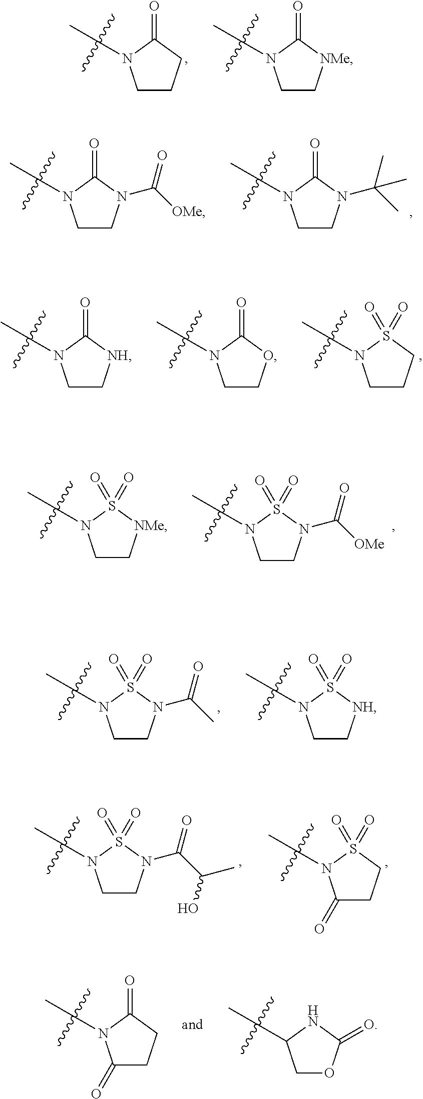

Stereochemistry

In some embodiments, the stereochemistry of the centre to which R² is bonded is S. In other embodiments, the stereochemistry of the centre to which R² is bonded is R.

Similarly, in some embodiments, the stereochemistry of the centre to which R³ is bonded is S. In other embodiments, the stereochemistry of the centre to which R³ is bonded is R.

In some embodiments, the relative stereochemistry between the centres to which R² and R³ are bonded is syn. In particular, the relative stereochemistry between the centres to which R² and R³ are bonded may be syn when R² and R³ are H; and R^{2′} and R³′ are independently C₁₋₁₀alkyl or C₃₋₁₀cycloalkyl. For example, the relative stereochemistry between the centres to which R² and R³ are bonded may be syn when R² and R³ are each H; and R²′ and R³′ are each methyl.

In other embodiments, the relative stereochemistry between the centres to which R² and R³ are bonded is anti. For example, the relative stereochemistry between the centres to which R² and R³ are bonded may be anti when R² and R³ are each H; and R²′ and R³′ are independently selected from C₁₋₁₀alkyl or C₃₋₁₀cycloalkyl.

Where present, the chiral centre(s) to which each R⁷ and each R⁸ is bonded may be independently selected from the R or S configurations.

Where present, the chiral centre(s) to which each R⁹ and each R¹⁰ is bonded may be independently selected from the R or S configurations. In some of the above embodiments, Y is:

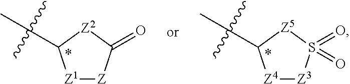

wherein the chiral centre indicated by an asterisk is of the R or S configuration, typically of the S configuration.

In further embodiments, p is 1 and Y is:

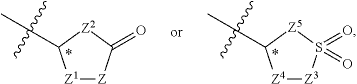

wherein the chiral centre indicated by an asterisk is of the R or S configuration, typically of the S configuration.

Specific Compounds

The invention provides the following specific compounds:

1-(3-((3R,5S)-3,5-dimethylpiperazin-1-yl)-4-methoxyphenyl)pyrrolidin-2-one;

1-(4-methoxy-3-(4-methylpiperazin-1-yl)phenyl)pyrrolidin-2-one;

1-(4-methoxy-3-(4-methylpiperazin-1-yl)phenyl)pyrrolidine-2,5-dione;

3-(34(3R,5S)-3,5-dimethylpiperazin-1-yl)-4-methoxyphenyl)oxazolidin-2-one;

3-(4-methoxy-3-(4-methylpiperazin-1-yl)phenyl)oxazolidin-2-one;

1-(4-methoxy-3-(4-methyl-1,4-diazepan-1-yl)phenyl)pyrrolidin-2-one;

2-(4-methoxy-3-(4-methyl-1,4-diazepan-1-yl)phenyl)-1,1-dioxoisothiazolidine;

2-(3-((3S,5R)-3,5-dimethylpiperazin-1-yl)-4-methoxyphenyl)-1,1-dioxoisothiazolidine;

1-(3-((3S,5R)-3,5-dimethylpiperazin-1-yl)-4-methoxybenzyl)pyrrolidin-2-one;

1-(7-((3S,5R)-3,5-dimethylpiperazin-1-yl)-2,3-dihydrobenzofuran-5-yl)pyrrolidin-2-one;

2-(3-((3S,5R)-3,5-dimethylpiperazin-1-yl)-4-fluorophenyl)-1,1-dioxoisothiazolidine;

2-(4-fluoro-3-(4-methyl-1,4-diazepan-1-yl)phenyl)-1,1-dioxoisothiazolidine;

2-(4-fluoro-3-(4-methylpiperazin-1-yl)phenyl)-1,1-dioxoisothiazolidine;

1-(7-(4-methylpiperazin-1-yl)-2,3-dihydrobenzofuran-5-yl)pyrrolidin-2-one;

1-(4-fluoro-3-(4-methylpiperazin-1-yl)phenyl)pyrrolidin-2-one;

(S)-4-(4-methoxy-3-(4-methylpiperazin-1-yl)benzyl)oxazolidin-2-one;

1-(7-(4-methyl-1,4-diazepan-1-yl)-2,3-dihydrobenzofuran-5-yl)pyrrolidin-2-one;

1-(7-(4-methylpiperazin-1-yl)benzofuran-5-yl)pyrrolidin-2-one;

3-(7-(4-methylpiperazin-1-yl)benzofuran-5-yl)oxazolidin-2-one;

methyl 5-(7-(4-methylpiperazin-1-yl)benzofuran-5-yl)-1,1-dioxo-1,2,5-thiadiazolidine-2-carboxylate;

3-(7-((3S,5R)-3,5-dimethylpiperazin-1-yl)benzofuran-5-yl)oxazolidin-2-one;

2-(7-(4-methylpiperazin-1-yl)benzofuran-5-y l)-1,1-dioxo-1,2,5-thiadiazolidine;

1-tert-butyl-3-(4-methoxy-3-(4-methylpiperazin-1-yl)phenyl)imidazolidin-2-one; and pharmaceutically acceptable derivatives thereof.

In another embodiment, the invention provides the following specific compounds:

3-(4-(4-methylpiperazin-1-yl)benzofuran-6-yl)oxazolidin-2-one;

3-(4-(4-methylpiperazin-1-yl)furo[3,2-c]pyridin-6-yl)oxazolidin-2-one;

3-(7-(4-methylpiperazin-1-yl)furo[2,3-c]pyridin-5-yl)oxazolidin-2-one;

2-methyl-5-[4-(4-methylpiperazin-1-yl)-1-benzofuran-6-yl]-1λ⁶,2,5-thiadiazolidine-1,1-dione;

2-(2-hydroxypropanoyl)-5-[7-(4-methylpiperazin-1-yl)-1-benzofuran-5-yl]-1λ⁶,2,5-thiadiazolidine-1,1-dione;

2-acetyl-5-[7-(4-methylpiperazin-1-yl)-1-benzofuran-5-yl]-1λ⁶,2,5-thiadiazolidine-1,1-dione;

3-(4-(4-methylpiperazin-1-yl)benzo[b]thiophen-6-yl)oxazolidin-2-one;

1-methyl-3-(4-(4-methyl piperazin-1-yl)benzo[b]thiophen-6-yl)imidazolidin-2-one;

2-methyl-5-[4-(4-methylpiperazin-1-yl)-1-benzothiophen-6-yl]-1λ⁶,2,5-thiadiazolidine-1,1-dione;

1-(4-methoxy-3-(4-methylpiperazin-1-yl)phenyl)-4,4-dimethylimidazolidin-2-one;

2-(7-(4-methylpiperazin-1-yl)-2,3-dihydrobenzofuran-5-yl)-1,1-dioxothiazolidine;

and pharmaceutically acceptable derivatives thereof.

In another embodiment, the invention provides the following specific compounds:

3-(7-(4-methylpiperazin-1-yl)furo[2,3-c]pyridin-5-yl)oxazolidin-2-one;

3-[7-(4-Methylpiperazin-1-yl)furo[2,3-c]pyridin-5-yl]-5-phenyl-1,3-oxazolidin-2-one

3-[7-(4-Methylpiperazin-1-yl)furo[2,3-c]pyridin-5-yl]-5-phenyl-1,3-oxazolidin-2-one hydrochloride

1-(7-(4-Methylpiperazin-1-yl)furo[2,3-c]pyridin-5-yl)-3-phenylimidazolidin-2-one

1-(7-(4-Methylpiperazin-1-yl)furo[2,3-c]pyridin-5-yl)-3-phenylimidazolidin-2-one hydrochloride

1-[7-(4-Methylpiperazin-l-yl)furo[2,3-c]pyridin-5-yl]pyrrolidin-2-one;

and pharmaceutically acceptable derivatives thereof.

In another embodiment, the invention provides the following specific compounds:

1-phenyl-3-(7-(piperazin-l-yl)furo[2,3-c]pyridin-5-yl)imidazolidin-2-one;

1-(7-((3R,5S)-3,5-dimethylpiperazin-1-yl)furo[2,3-c]pyridin-5-yl)-3-phenylimidazolidin-2-one;

1-(4-methoxyphenyl)-3-(7-(4-methylpiperazin-1-yl)furo[2,3-c]pyridin-5-yl)imidazolidin-2-one;

1-(7-(4-methylpiperazin-1-yl)furo[2,3-c]pyridin-5-yl)-3-(p-tolyl)imidazolidin-2-one;

1-(4-chlorophenyl)-3-(7-(4-methylpiperazin-1-yl)furo[2,3-c]pyridin-5-yl)imidazolidin-2-one;

1-(3,4-dichlorophenyl)-3-(7-(4-methylpiperazin-1-yl)furo[2,3-c]pyridin-5-yl)imidazolidin-2-one;

2-(7-(4-methylpiperazin-1-yl)furo[2,3-c]pyridin-5-yl)-5-phenyl-1,2,5-thiadiazolide 1,1-dioxide;

1-(5-methoxy-6-(4-methylpiperazin-1-yl)pyridin-2-yl)-3-phenylimidazolidin-2-one;

1-(5-methoxy-6-(4-methylpiperazin-1-yl)pyridin-2-yl)-3-(4-methoxyphenyl)imidazolidin-2-one;

1-(4-chlorophenyl)-3-(5-methoxy-6-(4-methylpiperazin-1-yl)pyridin-2-yl)imidazolidin-2-one;

1-(4-methoxy-3-(4-methylpiperazin-1-yl)phenyl)-3-phenylimidazolidin-2-one;

1-(4-chlorophenyl)-3-(4-methoxy-3-(4-methylpiperazin-1-yl)phenyl)imidazolidin-2-one;

2-(5-methoxy-6-(4-methylpiperazin-1-yl)pyridin-2-yl)-5-phenyl-1,2,5-thiadiazolidine 1,1-dioxide;

2-(4-chlorophenyl)-5-(5-methoxy-6-(4-methylpiperazin-1-yl)pyridin-2-yl)-1,2,5-thiadiazolidine 1,1-dioxide;

2-(5-methoxy-6-(4-methylpiperazin-1-yl)pyridin-2-yl)-5-(4-methoxyphenyl)-1,2,5-thiadiazolidine 1,1-dioxide;

and pharmaceutically acceptable derivatives thereof.

Chemical Groups

Halo

The term “halogen” (or “halo”) includes fluorine, chlorine, bromine and iodine.

Alkyl, alkylene, alkenyl, alkynyl, cycloalkyl etc.

The terms “alkyl”, “alkylene”, “alkenyl” or “alkynyl” are used herein to refer to both straight and branched chain acyclic forms. Cyclic analogues thereof are referred to as cycloalkyl, etc.

The term “alkyl” includes monovalent, straight or branched, saturated, acyclic hydrocarbyl groups. In one embodiment alkyl is C₁₋₁₀alkyl, in another embodiment C₁₋₆alkyl, in another embodiment C₁₋₄alkyl, such as methyl, ethyl, n-propyl, i-propyl or t-butyl groups.

The term “cycloalkyl” includes monovalent, saturated, cyclic hydrocarbyl groups. In one embodiment cycloalkyl is C₃₋₁₀cycloalkyl, in another embodiment C₃₋₆cycloalkyl such as cyclopentyl and cyclohexyl.

The term “alkoxy” means alkyl-O—.

The term “alkylamino” means alkyl-NH—.

The term “alkylthio” means alkyl-S(O)ₜ—, wherein t is defined below.

The term “alkenyl” includes monovalent, straight or branched, unsaturated, acyclic hydrocarbyl groups having at least one carbon-carbon double bond and, in one embodiment, no carbon-carbon triple bonds. In one embodiment alkenyl is C₂₋₁₀alkenyl, in another embodiment C₂₋₆alkenyl, in another embodiment C₂₋₄alkenyl.

The term “cycloalkenyl” includes monovalent, partially unsaturated, cyclic hydrocarbyl groups having at least one carbon-carbon double bond and, in one embodiment, no carbon-carbon triple bonds. In one embodiment cycloalkenyl is C₃₋₁₀cycloalkenyl, in another embodiment C₅₋₁₀cycloalkenyl, e.g. cyclohexenyl or benzocyclohexyl.

The term “alkynyl” includes monovalent, straight or branched, unsaturated, acyclic hydrocarbyl groups having at least one carbon-carbon triple bond and, in one embodiment, no carbon-carbon double bonds. In one embodiment, alkynyl is C₂₋₁₀alkynyl, in another embodiment C₂₋₆alkynyl, in another embodiment C₂₋₄alkynyl.

The term “alkylene” includes divalent, straight or branched, saturated, acyclic hydrocarbyl groups. In one embodiment alkylene is C₁₋₁₀alkylene, in another embodiment C₁₋₆alkylene, in another embodiment C₁₋₄alkylene, such as methylene, ethylene, n-propylene, i-propylene or t-butylene groups.

The term “alkenylene” includes divalent, straight or branched, unsaturated, acyclic hydrocarbyl groups having at least one carbon-carbon double bond and, in one embodiment, no carbon-carbon triple bonds. In one embodiment alkenylene is C₂₋₁₀alkenylene, in another embodiment C₂₋₆alkenylene, in another embodiment C₂₋₄alkenylene.

Heteroalkyl etc.

The term “heteroalkyl” includes alkyl groups in which up to three carbon atoms, in one embodiment up to two carbon atoms, in another embodiment one carbon atom, are each replaced independently by O, S(O)ₜ or N, provided at least one of the alkyl carbon atoms remains. The heteroalkyl group may be C-linked or hetero-linked, i.e. it may be linked to the remainder of the molecule through a carbon atom or through O, S(O)ₜ or N, wherein t is defined below.

The term “heterocycloalkyl” includes cycloalkyl groups in which up to three carbon atoms, in one embodiment up to two carbon atoms, in another embodiment one carbon atom, are each replaced independently by O, S(O)ₜ or N, provided at least one of the cycloalkyl carbon atoms remains. Examples of heterocycloalkyl groups include oxiranyl, thiaranyl, aziridinyl, oxetanyl, thiatanyl, azetidinyl, tetrahydrofuranyl, tetrahydrothiophenyl, pyrrolidinyl, tetrahydropyranyl, tetrahydrothiopyranyl, piperidinyl, 1,4-dioxanyl, 1,4-oxathianyl, morpholinyl, 1,4-dithianyl, piperazinyl, 1,4-azathianyl, oxepanyl, thiepanyl, azepanyl, 1,4-dioxepanyl, 1,4-oxathiepanyl, 1,4-oxaazepanyl, 1,4-dithiepanyl, 1,4-thieazepanyl and 1,4-diazepanyl. The heterocycloalkyl group may be C-linked or N-linked, i.e. it may be linked to the remainder of the molecule through a carbon atom or through a nitrogen atom.

The term “heteroalkenyl” includes alkenyl groups in which up to three carbon atoms, in one embodiment up to two carbon atoms, in another embodiment one carbon atom, are each replaced independently by O, S(O)ₜ or N, provided at least one of the alkenyl carbon atoms remains. The heteroalkenyl group may be C-linked or hetero-linked, i.e. it may be linked to the remainder of the molecule through a carbon atom or through O, S(O)ₜ or N.

The term “heterocycloalkenyl” includes cycloalkenyl groups in which up to three carbon atoms, in one embodiment up to two carbon atoms, in another embodiment one carbon atom, are each replaced independently by O, S(O)ₜ or N, provided at least one of the cycloalkenyl carbon atoms remains. Examples of heterocycloalkenyl groups include 3,4-dihydro-2H-pyranyl, 5-6-dihydro-2H-pyranyl, 2H-pyranyl, 1,2,3,4-tetrahydropyridinyl and 1,2,5,6-tetrahydropyridinyl. The heterocycloalkenyl group may be C-linked or N-linked, i.e. it may be linked to the remainder of the molecule through a carbon atom or through a nitrogen atom.

The term “heteroalkynyl” includes alkynyl groups in which up to three carbon atoms, in one embodiment up to two carbon atoms, in another embodiment one carbon atom, are each replaced independently by O, S(O)ₜ or N, provided at least one of the alkynyl carbon atoms remains. The heteroalkynyl group may be C-linked or hetero-linked, i.e. it may be linked to the remainder of the molecule through a carbon atom or through O, S(O)ₜ or N.

The term “heteroalkylene” includes alkylene groups in which up to three carbon atoms, in one embodiment up to two carbon atoms, in another embodiment one carbon atom, are each replaced independently by O, S(O), or N, provided at least one of the alkylene carbon atoms remains.

The term “heteroalkenylene” includes alkenylene groups in which up to three carbon atoms, in one embodiment up to two carbon atoms, in another embodiment one carbon atom, are each replaced independently by O, S(O), or N, provided at least one of the alkenylene carbon atoms remains.

Aryl

The term “aryl” includes monovalent, aromatic, cyclic hydrocarbyl groups, such as phenyl or naphthyl (e.g. 1-naphthyl or 2-naphthyl). In general, the aryl groups may be monocyclic or polycyclic fused ring aromatic groups. Preferred aryl are C₆-C₁₄aryl.

Other examples of aryl groups are monovalent derivatives of aceanthrylene, acenaphthylene, acephenanthrylene, anthracene, azulene, chrysene, coronene, fluoranthene, fluorene, as-indacene, s-indacene, indene, naphthalene, ovalene, perylene, phenalene, phenanthrene, picene, pleiadene, pyrene, pyranthrene and rubicene.

The term “arylalkyl” means alkyl substituted with an aryl group, e.g. benzyl.

Heteroaryl

The term “heteroaryl” includes aryl groups in which one or more carbon atoms are each replaced by heteroatoms independently selected from O, S, N and NR^{N}, where R^{N} is defined below (and in one embodiment is H or alkyl (e.g. C₁₋₁₀alkyl)).

In general, the heteroaryl groups may be monocyclic or polycyclic (e.g. bicyclic) fused ring heteroaromatic groups. Typically, heteroaryl groups contain 5-14 ring members (preferably 5-10 members) wherein 1, 2, 3 or 4 ring members are independently selected from O, S, N and NR^{N}. In one embodiment, a heteroaryl group may be 5, 6, 9 or 10 membered, e.g. 5-membered monocyclic, 6-membered monocyclic, 9-membered fused-ring bicyclic or 10-membered fused-ring bicyclic.

Monocyclic heteroaromatic groups include heteroaromatic groups containing 5-6 ring members wherein 1, 2, 3 or 4 ring members are independently selected from O, S , N or NR^{N}.

In one embodiment, 5-membered monocyclic heteroaryl groups contain 1 ring member which is an —NR^{N}— group, an —O— atom or an —S— atom and, optionally, 1-3 ring members (e.g. 1 or 2 ring members) which are ═N— atoms (where the remainder of the 5 ring members are carbon atoms).

Examples of 5-membered monocyclic heteroaryl groups are pyrrolyl, furanyl, thiophenyl, pyrazolyl, imidazolyl, isoxazolyl, oxazolyl, isothiazolyl, thiazolyl, 1,2,3 triazolyl, 1,2,4 triazolyl, 1,2,3 oxadiazolyl, 1,2,4 oxadiazolyl, 1,2,5 oxadiazolyl, 1,3,4 oxadiazolyl, 1,3,4 thiadiazolyl, pyridyl, pyrimidinyl, pyridazinyl, pyrazinyl, 1,3,5 triazinyl, 1,2,4 triazinyl, 1,2,3 triazinyl and tetrazolyl.

Examples of 6-membered monocyclic heteroaryl groups are pyridinyl, pyridazinyl, pyrimidinyl and pyrazinyl.

In one embodiment, 6-membered monocyclic heteroaryl groups contain 1 or 2 ring members which are ═N— atoms (where the remainder of the 6 ring members are carbon atoms).

Bicyclic heteroaromatic groups include fused-ring heteroaromatic groups containing 9-14 ring members wherein 1, 2, 3, 4 or more ring members are independently selected from O, S, N or NR^{N}.

In one embodiment, 9-membered bicyclic heteroaryl groups contain 1 ring member which is an —NR^{N}— group, an —O— atom or an —S— atom and, optionally, 1-3 ring members (e.g. 1 or 2 ring members) which are ═N— atoms (where the remainder of the 9 ring members are carbon atoms).

Examples of 9-membered fused-ring bicyclic heteroaryl groups are benzofuranyl, benzothiophenyl, indolyl, benzimidazolyl, indazolyl, benzotriazolyl, pyrrolo[2,3-b]pyridinyl, pyrrolo[2,3-c]pyridinyl, pyrrolo[3,2-c]pyridinyl, pyrrolo[3,2-b]pyridinyl, imidazo[4,5-b]pyridinyl, imidazo[4,5-c]pyridinyl, pyrazolo[4,3-d]pyridinyl, pyrazolo[4,3-c]pyridinyl, pyrazolo[3,4-c]pyridinyl, pyrazolo[3,4-b]pyridinyl, isoindolyl, indazolyl, purinyl, indolininyl, imidazo[1,2-a]pyridinyl, imidazo[1,5-a]pyridinyl, pyrazolo[1,2-a]pyridinyl, pyrrolo[1,2-b]pyridazinyl and imidazo[1,2-c]pyrimidinyl.

In one embodiment, 10-membered bicyclic heteroaryl groups contain 1-3 ring members which are ═N— atoms (where the remainder of the 10 ring members are carbon atoms).

Examples of 10-membered fused-ring bicyclic heteroaryl groups are quinolinyl, isoquinolinyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrazino[2,3-b]pyrazinyl and pyrimido[4,5-d]pyrimidinyl.

The term “heteroarylalkyl” means alkyl substituted with a heteroaryl group.

General

Unless indicated explicitly otherwise, where combinations of groups are referred to herein as one moiety, e.g. arylalkyl, the last mentioned group contains the atom by which the moiety is attached to the rest of the molecule.

Where reference is made to a carbon atom of an alkyl group or other group being replaced by O, S (O), or N, what is intended is that:

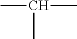

is replaced by

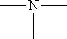

—CH′ is replaced by —N═;

═C—H is replaced by ≡EN; or

—CH₂— is replaced by —O—, —S(O)ₜ— or —NR^{N}—.

By way of clarification, in relation to the above mentioned heteroatom containing groups (such as heteroalkyl etc.), where a numerical of carbon atoms is given, for instance C₃₋₆heteroalkyl, what is intended is a group based on C₃₋₆alkyl in which one of more of the 3-6 chain carbon atoms is replaced by O, S (O), or N. Accordingly, a C₃₋₆heteroalkyl group, for example, will contain less than 3-6 chain carbon atoms.

Where mentioned above, R^{N} is H, alkyl, cycloalkyl, aryl, heteroaryl, —C(O)-alkyl, —C(O)-aryl, —C(O)-heteroaryl, —SO)ₜ-aryl or —S(O)ₜ-heteroaryl. R^{N} may, in particular, be H, alkyl (e.g. C₁₋₆alkyl) or cycloalkyl (e.g. C₃₋₆cycloalkyl).

Where mentioned above, t is independently 0, 1 or 2, for example 2. Typically, t is 0.

Where a group has at least 2 positions which may be substituted, the group may be substituted by both ends of an alkylene or heteroalkylene chain to form a cyclic moiety.

Substituents

Optionally substituted groups of the compounds of the invention (e.g. alkyl, cycloalkyl, alkoxy, alkenyl, cycloalkenyl, alkynyl, alkylene, alkenylene, heteroalkyl, heterocycloalkyl, heteroalkenyl, heterocycloalkenyl, heteroalkynyl, heteroalkylene, heteroalkenylene, aryl, arylalkyl, arylheteroalkyl, heteroaryl, heteroarylalkyl or heteroarylheteroalkyl groups etc.) may be substituted or unsubstituted, in one embodiment unsubstituted. Typically, substitution involves the notional replacement of a hydrogen atom with a substituent group, or two hydrogen atoms in the case of substitution by αO.

Where substituted, there will generally be 1 to 3 substituents, in one embodiment 1 or 2 substituents, in one embodiment 1 substituent.

The optional substituent(s) is/are independently halogen, trihalomethyl, trihaloethyl, —NO₂, —CN, —N⁺(C₁₋₆alkyl)₂O⁻, —CO₂H, —CO₂C₁₋₆alkyl, —SO₃H, —SOC₁₋₆alkyl, —SO₂C₁₋₆alkyl, —SO₃C₁₋₆alkyl, —OC(═O)OC₁₋₆alkyl, —C(═O)H, —C(═O)C₁₋₆alkyl, —OC(═O)C₁₋₆alkyl, ═O, —N(C₁₋₆alkyl)₂, —C(═O)NH₂, —C(═O)N(C₁₋₆alkyl)₂, —N(C₁₋₆alkyl)C(═O)O(C₁₋₆alkyl), —N(C₁₋₆alkyl)C(═O)N(C₁₋₆alkyl)₂, —OC(═O)N(C₁₋₆alkyl)₂, —N(C₁₋₆alkyl)C(═O)C₁₋₆alkyl, —C(═S)N(C₁₋₆alkyl)₂, —N(C₁₋₆alkyl)C(═S)C₁₋₆alkyl, —SO₂N(C₁₋₆alkyl)₂, —N(C₁₋₆alkyl)SO₂C₁₋₆alkyl, —N(C₁₋₆alkyl)C(═S)N(C₁₋₆alkyl)₂, —N(C₁₋₆alkyl)SO₂N (C₁₋₆alkyl)₂, —C₁₋₆alkyl, —C₁₋₆heteroalkyl, —C₃₋₆cycloalkyl, —C₃₋₆heterocycloalkyl, —C₂₋₆alkenyl, —C₂₋₆heteroalkenyl, —C₃₋₆cycloalkenyl, —C₃₋₆heterocycloalkenyl, —C₂₋₆alkynyl, —C₂₋₆heteroalkynyl, —Z^{u}—C₁₋₆alkyl, —Z^{u}—C₃₋₆cycloalkyl, —Z^{u}—C₂₋₆alkenyl, —Z^{u}—C₃₋₆cycloalkenyl or —Z^{u}—C₂₋₆alkynyl, wherein
Z^{u} is independently O, S, NH or N(C₁₋₆alkyl).

In another embodiment, the optional substituent(s) is/are independently halogen, trihalomethyl, trihaloethyl, —NO₂, —CN, —N⁺(C_{1,6}alkyl)₂O⁻, —CO₂H, —SO₃H, —SOC₁₋₆alkyl, —SO₂C₁₋₆alkyl, —C(═O)H, —C(═O)C₁₋₆alkyl, ═O, —N(C₁₋₆alkyl)₂, —C(═O)NH₂, —C₁₋₆alkyl, —C₃₋₆cycloalkyl, —C₃₋₆heterocycloalkyl, —Z^{u}C₁₋₆alkyl or —Z^{u}—C₃₋₆cycloalkyl, wherein Z^{u} is defined above.

In another embodiment, the optional substituent(s) is/are independently halogen, trihalomethyl, —NO₂, —CN, —CO₂H, —C(═O)C₁₋₆alkyl, ═O, —N(C₁₋₆alkyl)₂, —C(═O)NH₂, —C₁₋₆alkyl, —C₃₋₆cycloalkyl, —C₃₋₆heterocycloalkyl, —Z^{u}C₁₋₆alkyl or —Z—C₃₋₆cycloalkyl, wherein Z^{u} is defined above.

In another embodiment, the optional substituent(s) is/are independently halogen, —NO₂, —CN, —-CO₂H, ═O, —N(C₁₋₆alkyl)₂, —C₁₋₆alkyl, —C₃₋₆cycloalkyl or —C₃₋₆heterocycloalkyl.

In another embodiment, the optional substituent(s) is/are independently halogen, ═O, —C₁₋₆alkyl, —C₃₋₆cycloalkyl or —C₃₋₆heterocycloalkyl.

Compounds of Formula (I) and Derivatives Thereof

As used herein, the terms “compounds of the invention” and “compound of formula (I)” etc. include pharmaceutically acceptable derivatives thereof and polymorphs, isomers and isotopically labelled variants thereof. Furthermore, the term “compounds of the invention” and “compound of formula (I)” etc include compounds of formula (Ia) and (Ib) and the embodiments thereof disclosed herein.

Pharmaceutically Acceptable Derivatives

The term “pharmaceutically acceptable derivative” includes any pharmaceutically acceptable salt, solvate, hydrate or prodrug of a compound of formula (I). In one embodiment, the pharmaceutically acceptable derivatives are pharmaceutically acceptable salts, solvates or hydrates of a compound of formula (I).

Pharmaceutically Acceptable Salts

The term “pharmaceutically acceptable salt” includes a salt prepared from pharmaceutically acceptable non-toxic acids or bases including inorganic or organic acids and bases.

Compounds of formula (I) which contain basic, e.g. amino, groups are capable of forming pharmaceutically acceptable salts with acids. In one embodiment, pharmaceutically acceptable acid addition salts of the compounds of formula (I) include, but are not limited to, those of inorganic acids such as hydrohalic acids (e.g. hydrochloric, hydrobromic and hydroiodic acid), sulfuric acid, nitric acid and phosphoric acids. In one embodiment, pharmaceutically acceptable acid addition salts of the compounds of formula (I) include, but are not limited to, those of organic acids such as aliphatic, aromatic, carboxylic and sulfonic classes of organic acids, examples of which include: aliphatic monocarboxylic acids such as formic acid, acetic acid, propionic acid or butyric acid; aliphatic hydroxy acids such as lactic acid, citric acid, tartaric acid or malic acid; dicarboxylic acids such as maleic acid or succinic acid; aromatic carboxylic acids such as benzoic acid, p-chlorobenzoic acid, phenylacetic acid, diphenylacetic acid or triphenylacetic acid; aromatic hydroxyl acids such as o-hydroxybenzoic acid, p-hydroxybenzoic acid, 1-hydroxynaphthalene-2-carboxylic acid or 3-hydroxynaphthalene-2-carboxylic acid; and sulfonic acids such as methanesulfonic acid, ethanesulfonic acid or benzenesulfonic acid. Other pharmaceutically acceptable acid addition salts of the compounds of formula (I) include, but are not limited to, those of glycolic acid, glucuronic acid, furoic acid, glutamic acid, anthranilic acid, salicylic acid, mandelic acid, embonic (pamoic) acid, pantothenic acid, stearic acid, sulfanilic acid, algenic acid and galacturonic acid. Wherein the compound of formula (I) comprises a plurality of basic groups, multiple centres may be protonated to provide multiple salts, e.g. di- or tri-salts of compounds of formula (I). For example, a hydrohalic acid salt of a compound of formula (I) as described herein may be a monohydrohalide, dihydrohalide or trihydrohalide, etc. In one embodiment, the salts include, but are not limited to those resulting from addition of any of the acids disclosed above. In one embodiment of the compound of formula (I), two basic groups form acid addition salts. In a further embodiment, the two addition salt counterions are the same species, e.g. dihydrochloride, dihydrosulphide etc. Typically, the pharmaceutically acceptable salt is a hydrochloride salt, such as a dihydrochloride salt.

Compounds of formula (I) which contain acidic, e.g. carboxyl, groups are capable of forming pharmaceutically acceptable salts with bases. In one embodiment, pharmaceutically acceptable basic salts of the compounds of formula (I) include, but are not limited to, metal salts such as alkali metal or alkaline earth metal salts (e.g. sodium, potassium, magnesium or calcium salts) and zinc or aluminium salts. In one embodiment, pharmaceutically acceptable basic salts of the compounds of formula (I) include, but are not limited to, salts formed with ammonia or pharmaceutically acceptable organic amines or heterocyclic bases such as ethanolamines (e.g. diethanolamine), benzylamines, N-methyl-glucamine, amino acids (e.g, lysine) or pyridine.

Hemisalts of acids and bases may also be formed, e.g. hemisulphate salts.

Pharmaceutically acceptable salts of compounds of formula (I) may be prepared by methods well-known in the art.

For a review of pharmaceutically acceptable salts, see Stahl and Wermuth, Handbook of Pharmaceutical Salts: Properties, Selection and Use (Wiley-VCH, Weinheim, Germany, 2002).

Solvates & Hydrates

The compounds of the invention may exist in both unsolvated and solvated forms. The term “solvate” includes molecular complexes comprising a compound of the invention and one or more pharmaceutically acceptable solvent molecules such as water or C₁₋₆ alcohols, e.g. ethanol. The term “hydrate” means a “solvate” where the solvent is water.

Prodrugs

The invention includes prodrugs of the compounds of formula (I). Prodrugs are derivatives of compounds of formula (I) (which may have little or no pharmacological activity themselves), which can, when administered in vivo, be converted into compounds of formula (I).

Prodrugs can, for example, be produced by replacing functionalities present in the compounds of formula (I) with appropriate moieties which are metabolized in vivo to form a compound of formula (I). The design of prodrugs is well-known in the art, as discussed in Bundgaard, Design of Prodrugs 1985 (Elsevier), The Practice of Medicinal Chemistry 2003, 2^{nd} Ed, 561-585 and Leinweber, Drug Metab. Res. 1987, 18: 379.

Examples of prodrugs of compounds of formula (I) are esters and amides of the compounds of formula (I). For example, where the compound of formula (I) contains a carboxylic acid group (—COOH), the hydrogen atom of the carboxylic acid group may be replaced in order to form an ester (e.g. the hydrogen atom may be replaced by C₁₋₆alkyl). Where the compound of formula (I) contains an alcohol group (—OH), the hydrogen atom of the alcohol group may be replaced in order to form an ester (e.g. the hydrogen atom may be replaced by —C(O)C₁₋₆alkyl. Where the compound of formula (I) contains a primary or secondary amino group, one or more hydrogen atoms of the amino group may be replaced in order to form an amide (e.g. one or more hydrogen atoms may be replaced by —C(O)C₁₋₆alkyl).

Amorphous & Crystalline Forms

The compounds of the invention may exist in solid states from amorphous through to crystalline forms. All such solid forms are included within the invention.

Isomeric Forms

Compounds of the invention may exist in one or more geometrical, optical, enantiomeric, diastereomeric and tautomeric forms, including but not limited to cis- and trans-forms, E- and Z-forms, R-, S- and meso-forms, keto- and enol-forms. All such isomeric forms are included within the invention. The isomeric forms may be in isomerically pure or enriched form, as well as in mixtures of isomers (e.g. racemic or diastereomeric mixtures).

Accordingly, the invention provides:
stereoisomeric mixtures of compounds of formula (I);
a diastereomerically enriched or diastereomerically pure isomer of a compound of formula (I); or
an enantiomerically enriched or enantiomerically pure isomer of a compound of formula (I).

Where appropriate, isomers can be separated from their mixtures by the application or adaptation of known methods (e.g. chromatographic techniques, resolution techniques and recrystallization techniques). Where appropriate, isomers can be prepared by the application or adaptation of known methods (e.g. asymmetric synthesis).

Isotopic Labeling

The invention includes pharmaceutically acceptable isotopically-labelled compounds of formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labelled compounds of formula (I), for example, those incorporating a radioactive isotope, are useful in drug and/or substrate tissue distribution studies. The radioactive isotopes ³H and ¹⁴C are particularly useful for this purpose in view of their ease of incorporation and ready means of detection.

Substitution with positron emitting isotopes, such as ¹¹C, ⁸¹F, ¹⁵O and ¹³N, can be useful in Positron Emission Topography (PET) studies for examining substrate receptor occupancy.

Isotopically-labelled compounds of formula (I) can generally be prepared by conventional techniques known to those skilled in the art or by processes analogous to those described herein using an appropriate isotopically-labelled reagent in place of the non-labelled reagent previously employed.

Treatment of Diseases and Conditions

Compounds of formula (I) have been found by the inventors to be useful as 5-HT_{1B} receptor modulators, typically as antagonists. The invention provides a compound of formula (I) for use in therapy. The invention further provides a pharmaceutical composition comprising a compound of formula (I) in combination with a pharmaceutically acceptable excipient.

The invention further provides a method for the treatment of a disease or condition mediated by 5-HT_{1B} receptors, comprising the step of administering a therapeutically effective amount of a compound of formula (I) to a patient. The invention also provides the use of a compound of formula (I) in the manufacture of a medicament for the treatment of a disease or condition mediated by 5-HT_{1B} receptors. The invention also provides a compound of formula (I) for use in treating a disease or condition mediated by 5-HT_{1B} receptors.

The invention also provides a crystal of the 5-HT_{1B} receptor and a compound of formula (I). Such crystals can be used for X-ray diffraction studies of 5-HT_{1B} receptor binding, e.g. to provide atomic structural information in order to aid rational design of further 5-HT_{1B} receptor ligands.

Preferred compounds of the invention have an IC₅₀ in the rat, guinea pig or human 5-I-IT_{1B} receptor assays described below of <100 μM, in one embodiment <10 μM, in another embodiment <1 μM, in another embodiment <100 nM and in another embodiment <10 nM. In particular, compounds of the invention have an IC₅₀ of <50 μM in the rat 5-HT_{1B} receptor assay described below, <50 μM in the guinea pig 5-HT_{1B} receptor assay described below or <1 μM in the human 5-HT_{1B} receptor assay described below.

The invention is useful for the treatment of a disease or condition mediated by 5-HT_{1B} receptors.

Diseases and conditions mediated by 5-HT_{1B} receptors comprise vascular diseases, such as cardiovascular diseases, peripheral vascular diseases and cerebrovascular diseases.

In particular, the disease or condition mediated by 5-HT_{1B} receptors may be a vascular disease selected from:

a) cardiovascular diseases, such as angina pectoris, coronary arteriosclerosis (chronic ischemic heart disease, asymptomatic ischemic heart disease and arteriosclerotic cardiovascular disease); heart failure, congestive heart failure, painless ischemic heart disease, myocardial ischemia, myocardial infarction and diseases that arise from thrombotic states in which the coagulation cascade is activated;

b) peripheral vascular diseases, including peripheral arterial disease, such as chronic arterial occlusion including arteriosclerosis, arteriosclerosis obliterans and thromboangiitis obliterans (Buerger's disease), macroangiopathy, microangiopathy, thrombophlebitis, phlebemphraxis, Raynaud's disease, Raynaud's syndrome, CREST syndrome, vascular claudication, disturbance of peripheral circulation function, peripheral circulation disorder, erectile dysfunction, male impotence, female sexual dysfunction, retinopathy, maculopathy, occlusion of the retinal artery, obstruction of central artery of retina, occlusion of retinal vein, neovascular maculopathy, edema, vasculitis, frostbite (cold injury), chilblain, gangrene, hypertension, pulmonary hypertension, portal hypertension, diabetic nephropathy, renal failure, vasospasm, acrocyanosis, ateriovenous fistula, arteriovenous malformations, chronic venous insufficiency, deep vein thrombosis, erythromelalgia, fibromuscular dysplasia, Klippel-Trenauney syndrome, lymphedema, lipedemia, varicose veins and vascular birthmark; and

c) cerebrovascular diseases, such as, migraine, cerebral ischemia, cerebral infarction, cerebral vasospasm and thrombotic stroke.

More particularly, the disease or condition mediated by 5-HT_{1B} receptors may be a vascular disease selected from acrocyanosis, angina, ateriovenous fistula, arteriovenous malformations, Buerger's disease, chronic venous insufficiency, deep vein thrombosis, erythromelalgia, fibromuscular dysplasia, gangrene, Klippel-Trenauney syndrome, lymphedema, lipedemia, myocardial ischemia, myocardial infarction, pulmonary hypertension, portal hypertension, Raynaud's syndrome, thrombosis, thrombophlebitis, varicose veins, vascular birthmark and vasculitis.

Typically, the disease or condition mediated by 5-HT_{1B} receptors is a vascular disease selected from angina, peripheral vascular disease, pulmonary hypertension, portal hypertension and Raynaud's syndrome.

In particular, the pulmonary hypertension may be pulmonary arterial hypertension.

Diseases and conditions mediated by 5-HT_{1B} receptors also comprise cancer. It is particularly contemplated that the cancer be associated with formation of solid tumors, including carcinomas, such as adenocarcinomas and epithelial carcinomas. Such cancers can include, but are not limited to, lung cancer, including non-small cell lung cancer and large cell carcinoma types, as well as small cell lung cancer; colon cancer, including colon metastasized to liver and including colorectal cancers; breast cancer; and ovarian cancer, as mentioned above. Cancers that can be associated with solid tumors further include, but are not limited to, kidney or renal cancers, including, for example, renal cell carcinomas; cancer of the bladder; liver cancer, including, for example, hepatocellular carcinomas; cancer of the gastrointestinal tract, including rectal, esophageal, pancreatic and stomach cancer; gynecological cancers, including cervical, uterine and endometrial cancers; prostate cancer or testicular cancer; nasopharyngeal cancer; thyroid cancer, for example, thyroid papillary carcinoma; cancer of the head, neck or brain; nervous system cancers, including neuroblastomas; skin cancers, including melanomas; and sarcomas (including, for example, osteosarcomas and Ewing's sarcomas). Carcinomas include, but are not limited to, adenocarcinomas and epithelial carcinomas. It is also contemplated herein that the cancer is a hematological malignancy. Hematological malignancies include, but are not limited to, leukemias, including, but not limited to, acute lymphoblastic leukemia (ALL), acute myeloid leukemia (AML), chronic myelogenous leukemia (CML), acute lymphoblastic or precursor lymphoblastic leukemia, chronic lymphocytic leukemia (CLL) and hairy cell leukemia; lymphomas, e.g., mature B cell neoplasms, mature T cell and natural killer (NK) cell neoplasms, Hodgkin's lymphoma, non-Hodgkin lymphoma, immunodeficiency-associated lymphoproliferative disorders and histiocytic and dendritic cell neoplasms, etc.; and myelomas, such as multiple myelomas. The disease or condition mediated by 5-HT_{1B} receptors may, in particular, be cancer of the bladder or prostate, particularly cancer of the bladder. Any mammal, preferably a human, may be treated according to the present invention.

Diseases and conditions mediated by 5-HT_{1B} receptors also comprise central nervous system (CNS) disorders, comprising, for example, anxiety disorder; including anxiety disorders such as panic disorder, panic disorder without agoraphobia, panic disorder with agoraphobia, agoraphobia without history of panic disorder, specific phobia, social phobia, social anxiety disorder, obsessive-compulsive disorder, posttraumatic stress disorder, avoidant personality disorder, borderline personality disorders, acute stress disorder, generalized anxiety disorder and generalized anxiety disorder due to a general medical condition; cognitive disorder, including cognitive disorders such as Alzheimer's disease, dementia, dementia due to Alzheimer's disease, dementia due to Parkinson's disease and Huntington's disease; mood disorder, including mood disorders such as a depressive disorder, such as, for example, major depressive disorder, dysthymic disorder, bipolar depression and/or bipolar mania, cyclothymic disorder, mood disorder due to a general medical condition, manic episode associated with bipolar disorder, and mixed episode associated with bipolar disorder, bipolar disorder wherein the bipolar depression and/or bipolar mania is bipolar II, or bipolar I with or without manic, depressive or mixed episodes; eating disorders, such as anorexia, bulimia and obesity; gastrointestinal disorders, motor disorders; cardiovascular regulation, pulmonary vasoconstriction, endocrine disorders, such as hyperprolactinaemia; vasospasm, jet lag, seizures, attention deficit hyperactivity disorder (ADHD), Tourette's Syndrome, tardive dyskinesia, blocking carbohydrate cravings, late luteal phase dysphoric disorder, tobacco withdrawal-associated symptoms, chemical dependencies and addictions (e.g., dependencies on, or addictions to, nicotine [and/or tobacco products], alcohol, benzodiazepines, barbiturates, opioids or cocaine), headache, stroke, traumatic brain injury (TBI), psychosis, epilepsy, COPD, sexual dysfunction of an animal, particularly a mammal, most particularly a human. The disease or condition mediated by 5-HT_{1B} receptors may, in particular, be gastrointestinal disorders and COPD.

Particular diseases or conditions mediated by 5-HT_{1B} receptors include angina, pulmonary hypertension, portal hypertension, Raynaud's syndrome, bladder cancer, prostate cancer, gastrointestinal disorders and COPD.

In particular, the pulmonary hypertension may be pulmonary arterial hypertension.

Therapeutic Definitions

As used herein, “treatment” includes curative and prophylactic treatment. As used herein, a “patient” means an animal, preferably a mammal, preferably a human, in need of treatment.

The amount of the compound of the invention administered should be a therapeutically effective amount where the compound or derivative is used for the treatment of a disease or condition and a prophylactically effective amount where the compound or derivative is used for the prevention of a disease or condition.

The term “therapeutically effective amount” used herein refers to the amount of compound needed to treat or ameliorate a targeted disease or condition. The term “prophylactically effective amount” used herein refers to the amount of compound needed to prevent a targeted disease or condition. The exact dosage will generally be dependent on the patient's status at the time of administration. Factors that may be taken into consideration when determining dosage include the severity of the disease state in the patient, the general health of the patient, the age, weight, gender, diet, time, frequency and route of administration, drug combinations, reaction sensitivities and the patient's tolerance or response to therapy. The precise amount can be determined by routine experimentation, but may ultimately lie with the judgement of the clinician. Generally, an effective dose will be from 0.01 mg/kg/day (mass of drug compared to mass of patient) to 1000 mg/kg/day, e.g. 1 mg/kg/day to 100 mg/kg/day. Compositions may be administered individually to a patient or may be administered in combination with other agents, drugs or hormones.

Administration & Formulation

General

For pharmaceutical use, the compounds of the invention may be administered as a medicament by enteral or parenteral routes, including intravenous, intramuscular, subcutaneous, transdermal, airway (aerosol), oral, intranasal, rectal, vaginal, urethral and topical (including buccal and sublingual) administration. The compounds of formula (I) should be assessed for their biopharmaceutical properties, such as solubility and solution stability (across pH), permeability, etc., in order to select the most appropriate dosage form and route of administration for treatment of the proposed indication.

The compounds of the invention may be administered as crystalline or amorphous products. The compounds of the invention may be administered alone or in combination with one or more other compounds of the invention or in combination with one or more other drugs (or as any combination thereof). Generally, they will be administered as a formulation in association with one or more pharmaceutically acceptable excipients. The term “excipient” includes any ingredient other than the compound(s) of the invention which may impart either a functional (e.g drug release rate controlling) and/or a non-functional (e.g. processing aid or diluent) characteristic to the formulations. The choice of excipient will to a large extent depend on factors such as the particular mode of administration, the effect of the excipient on solubility and stability and the nature of the dosage form.

Typical pharmaceutically acceptable excipients include:
diluents, e.g. lactose, dextrose, sucrose, mannitol, sorbitol, cellulose and/or glycine;
lubricants, e.g. silica, talcum, stearic acid, its magnesium or calcium salt and/or polyethyleneglycol;
binders, e.g. magnesium aluminum silicate, starch paste, gelatin, tragacanth, methylcellulose, sodium carboxymethylcellulose and/or polyvinylpyrrolidone;
disintegrants, e.g. starches, agar, alginic acid or its sodium salt, or effervescent mixtures; and/or
absorbants, colorants, flavors and/or sweeteners.

A thorough discussion of pharmaceutically acceptable excipients is available in Gennaro, Remington: The Science and Practice of Pharmacy 2000, 20th edition (ISBN: 0683306472).

Accordingly, in one embodiment, the present invention provides a pharmaceutical composition comprising a compound of formula (I) and a pharmaceutically acceptable excipient.

Oral Administration

The compounds of the invention may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid plugs, solid microparticulates, semi-solid and liquid (including multiple phases or dispersed systems) such as tablets; soft or hard capsules containing multi- or nano-particulates, liquids (e.g. aqueous solutions), emulsions or powders; lozenges (including liquid-filled); chews; gels; fast dispersing dosage forms; films; ovules; sprays; and buccal/mucoadhesive patches.

Formulations suitable for oral administration may also be designed to deliver the compounds of formula (I) in an immediate release manner or in a rate-sustaining manner, wherein the release profile can be delayed, pulsed, controlled, sustained, or delayed and sustained or modified in such a manner which optimises the therapeutic efficacy of the said compounds. Means to deliver compounds in a rate-sustaining manner are known in the art and include slow release polymers that can be formulated with the said compounds to control their release.

Examples of rate-sustaining polymers include degradable and non-degradable polymers that can be used to release the said compounds by diffusion or a combination of diffusion and polymer erosion. Examples of rate-sustaining polymers include hydroxypropyl methylcellulose, hydroxypropyl cellulose, methyl cellulose, ethyl cellulose, sodium carboxymethyl cellulose, polyvinyl alcohol, polyvinyl pyrrolidone, xanthum gum, polymethacrylates, polyethylene oxide and polyethylene glycol.

Liquid (including multiple phases and dispersed systems) formulations include emulsions, suspensions, solutions, syrups and elixirs. Such formulations may be presented as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Liang and Chen, Expert Opinion in Therapeutic Patents 2001, 11(6): 981-986 .

The formulation of tablets is discussed in H. Lieberman and L. Lachman, Pharmaceutical Dosage Forms: Tablets 1980, vol. 1 (Marcel Dekker, New York).

Parenteral Administration

The compounds of the invention can be administered parenterally.

The compounds of the invention may be administered directly into the blood stream, into subcutaneous tissue, into muscle, or into an internal organ. Suitable means for administration include intravenous, intraarterial, intrathecal, intraventricular, intraurethral, intrasternal, intracranial, intramuscular, intrasynovial and subcutaneous. Suitable devices for administration include needle (including microneedle) injectors, needle-free injectors and infusion techniques.

Parenteral formulations are typically aqueous or oily solutions. Where the solution is aqueous, excipients such as sugars (including but not restricted to glucose, mannitol, sorbitol, etc.) salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water (WFI).

Parenteral formulations may include implants derived from degradable polymers such as polyesters (i.e. polylactic acid, polylactide, polylactide-co-glycolide, polycapro-lactone, polyhydroxybutyrate), polyorthoesters and polyanhydrides. These formulations may be administered via surgical incision into the subcutaneous tissue, muscular tissue or directly into specific organs.

The preparation of parenteral formulations under sterile conditions, for example, by lyophilization, may readily be accomplished using standard pharmaceutical techniques well known to those skilled in the art.

The solubility of compounds of formula (I) used in the preparation of parenteral solutions may be increased by the use of appropriate formulation techniques, such as the incorporation of co-solvents and/or solubility-enhancing agents such as surfactants, micelle structures and cyclodextrins.

Inhalation & Intranasal Administration

The compounds of the invention can be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler, as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol or magnesium stearate. The lactose may be anhydrous or in the form of the monohydrate, preferably the latter. Other suitable excipients include dextran, glucose, maltose, sorbitol, xylitol, fructose, sucrose and trehalose.

Formulations for inhaled/intranasal administration may be formulated to be immediate and/or modified release using, for example, poly(lactic-co-glycolic acid) (PGLA). Modified release formulations include delayed-, sustained-, pulsed-, controlled-, targeted and programmed release.

Transdermal Administration

Suitable formulations for transdermal application include a therapeutically effective amount of a compound of the invention with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Combination Therapy

The compound of formula (I) may be administered alone or may be administered in combination with another therapeutic agent (i.e. a different agent to the compound of formula (I)). Preferably, the compound of the invention and the other therapeutic agent are administered in a therapeutically effective amount.

The compound of the present invention may be administered either simultaneously with, or before or after, the other therapeutic agent. The compound of the present invention may be administered separately, by the same or different route of administration, or together in the same pharmaceutical composition.

In one embodiment, the invention provides a product comprising a compound of formula (I) and another therapeutic agent as a combined preparation for simultaneous, separate or sequential use in therapy. In one embodiment, the therapy is the treatment of a disease or condition mediated by 5-HT_{1B} receptors. Products provided as a combined preparation include a composition comprising the compound of formula (I) and the other therapeutic agent together in the same pharmaceutical composition, or the compound of formula (I) and the other therapeutic agent in separate form, e.g. in the form of a kit.

In one embodiment, the invention provides a pharmaceutical composition comprising a compound of formula (I) and another therapeutic agent. Optionally, the pharmaceutical composition may comprise a pharmaceutically acceptable excipient, as described above in “Administration & Formulation”.

In one embodiment, the invention provides a kit comprising two or more separate pharmaceutical compositions, at least one of which contains a compound of formula (I). In one embodiment, the kit comprises means for separately retaining said compositions, such as a container, divided bottle or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

Accordingly, the invention provides the use of a compound of formula (I) in the manufacture of a medicament for treating a disease or condition mediated by 5-HT_{1B} receptors, wherein the medicament is prepared for administration with another therapeutic agent. The invention also provides the use of another therapeutic agent in the manufacture of medicament for treating a disease or condition mediated by 5-HT_{1B} receptors, wherein the medicament is prepared for administration with a compound of formula (I).

The invention also provides a compound of formula (I) for use in a method of treating a disease or condition mediated by 5-HT_{1B} receptors, wherein the compound of formula (I) is prepared for administration with another therapeutic agent. The invention also provides another therapeutic agent for use in a method of treating a disease or condition mediated by 5-HT_{1B} receptors, wherein the other therapeutic agent is prepared for administration with a compound of formula (I). The invention also provides a compound of formula (I) for use in a method of treating a disease or condition mediated by 5-HT_{1B} receptors, wherein the compound of formula (I) is administered with another therapeutic agent. The invention also provides another therapeutic agent for use in a method of treating a disease or condition mediated by 5-HT_{1B} receptors, wherein the other therapeutic agent is administered with a compound of formula (I).

The invention also provides the use of a compound of formula (I) in the manufacture of a medicament for treating a disease or condition mediated by 5-HT_{1B} receptors, wherein the patient has previously (e.g. within 24 hours) been treated with another therapeutic agent. The invention also provides the use of another therapeutic agent in the manufacture of a medicament for treating a disease or condition mediated by 5-HT_{1B} receptors, wherein the patient has previously (e.g. within 24 hours) been treated with a compound of formula (I).

In one embodiment, the other therapeutic agent is selected from:
(i) blood pressure lowering therapies, comprising, for example, a) Angiotensin-converting enzyme (ACE) inhibitors, such as benazepril, captopril, cilazapril, enalapril, fosinopril, lisinopril, perindopril, quinapril, ramipril and trandolapril; b) Angiotensin Receptor Blockers, such as candesartan, eprosartan, irbesartan, losartan, olmesartan, telmisartan and valsartan; c) Calcium-channel blockers, such as amlodipine, diltiazem, felodipine, isradipine, lacidipine, lercanidipine, nicardipine, nifedipine, nisoldipine and verapamil; d) Diuretics, such as bendroflumethiazide (bendrofluazide), chlorothiazide, chlorthalidone, cyclopenthiazide, furosemide, hydrochlorothiazide indapamide, metolazone and torsemide; e) Beta-blockers, such as acebutolol, atenolol, betaxolol, bisoprolol, metoprolol, nadolol, oxprenolol, pindolol, propranolol, sotalol and timolol; f) methyldopa or alpha blockers; g) endothelin receptor antagonists such as bosentan, darusentan, enrasentan, tezosentan, atrasentan, ambrisentan sitaxsentan; h) smooth muscle relaxants such as PDE5 inhibitors (indirect-acting), minoxidil and diazoxide (direct-acting); i) alpha receptor blockers, such as doxazosin, terazosin, alfuzosin, tamsulosin; and j) central alpha agonists, such as clonidine.
(ii) Raynaud's syndrome therapies, comprising, for example, the above blood-pressure lowering drugs and a) Alpha-adrenoceptor-blocking drugs, such as Prazosin and Moxisylyte; b) Peripheral vasodilators, such as Cilostazol, Cinnarizine, Inositol nicotinate and Naftidrofuryl oxalate; c) vasodilators, such as Pentoxifylline (oxpentoxifylline), Sildenafil and Glyceryl trinitrate (GTN) as found in Coro-nitro, Glytrin, Nitromin, Minitram, Percutol, Nitrolingual, Nitro-Dur, Deponit, Transiderm Nitro, Sustac, Nitrocontin and Suscard; d) Prostaglandins, such as Beraprost, Alprostadil, Epoprostenol and Iloprost; and e) Selective serotonin re-uptake inhibitors, such as Fluoxetine;
(iii) angina therapies, comprising, for example, the above vasodilators and a) Isosorbide dinitrate (ISDN), as found in Angitac, Sorbid, Isoket, Sorbitrate, Sorbichew, Isordil and Cedocard; and b) Isosorbide mononitrate (ISMN), as found in Isotrate, Chemydur, Imdur, Isib, Isotard, MCR, Modisal, Monomax, Monosorb, Imazin, Elantan, Ismo, Monit and Mono-Cedocard;
(iv) cholesterol lowering therapies, comprising, for example, a) statins, such as atorvastatin, fluvastatin, lovastatin, pravastatin, rosuvastatin and simvastatin; b) Anion-exchange resins such as colestyramine (cholestyramine) and colestipol; c) Fibrates, such as bezafibrate, ciprofibrate, fenofibrate and gemfibrozil; d) cholesteryl ester transfer protein inhibitors, such as torcetrapib; and d) others, such as Nicotinic acid, Ezetimibe, cholesterol absorption inhibitors and Fish oils; and
(v) peripheral vascular disease therapies, comprising, for example, a) cilostazol (commercial name: Pletaal) and prostaglandin (PG) preparations (commercial names: Dorner, Opalmon, etc.) having a vasodilative effect as well as an antiplatelet effect; b) ticlopidine, mainly having an antiplatelet effect (commercial name: Panaldine); c) sarpogrelate (commercial name: Anplag) and ethyl icosapentate (commercial name: Epadel); d) injectable preparations including prostaglandin El preparations and antithrombin preparations (commercial name: Argatroban).

In another embodiment, the other therapeutic agent is selected from chemotherapeutic agents, for example:
(i) alkylating agents, comprising, for example, busulfan, cisplatin, carboplatin, chlorambucil, cyclophosphamide, ifosfamide, dacarbazine (DTIC), mechlorethamine (nitrogen mustard), melphalan and temozolomide;
(ii) nitrosoureas, comprising, for example, carmustine (BCNU) and lomustine (CCNU);
(iii) antimetabolites, comprising, for example, 5-fluorouracil, capecitabine, 6-mercaptopurine, methotrexate, gemcitabine, cytarabine (ara-C), fludarabine and pemetrexed;
(iv) anthracyclines and related drugs, comprising, for example, daunorubicin, doxorubicin (Adriamycin), epirubicin, idarubicin and mitoxantrone;
(v) topoisomerase H inhibitors, comprising, for example, topotecan, irinotecan, etoposide (VP-16) and teniposide;
(vi) mitotic inhibitors, comprising, for example, taxanes (paclitaxel, docetaxel) and the vinca alkaloids (vinblastine, vincristine and vinorelbine); and
(vii) corticosteroid hormones, comprising, for example, prednisone and dexamethasone.

The chemotherapeutics may also be selected from other known chemotherapeutics, e.g. L-asparaginase, dactinomycin, thalidomide, tretinoin, imatinib (Gleevec), gefitinib (Iressa), erlotinib (Tarceva), rituximab (Rituxan), bevacizumab (Avastin), anti-estrogens (tamoxifen, fulvestrant), aromatase inhibitors (anastrozole, exemestane, letrozole), progestins (megestrol acetate), anti-androgens (bicalutamide, flutamide) and LHRH agonists (leuprolide, goserelin).

It is particularly contemplated that the chemotherapeutic agent can be, for example, a microtubule poison, a DNA alkylating agent, etc. Suitable microtubule poisons include, but are not limited to, paclitaxel. Suitable DNA alkylating agents include, e.g., carboplatin, etc.

In another embodiment, the other therapeutic agent is selected from:
(i) antidepressants, comprising, for example, amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, mirtazapine, nortriptyline, nefazodone, paroxetine, phenelzine, protriptyline, reboxetine, sertraline, sibutramine, thionisoxetine, tranylcypromaine, trazodone, trimipramine and venlafaxine;
(ii) atypical antipsychotics, comprising, for example, quetiapine and lithium;
(iii) antipsychotics, comprising, for example, amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutlypiperidine, pimozide, prochlorperazine, risperidone, sertindole, sulpiride, suproclone, suriclone, thioridazine, trifluoperazine, trimetozine, valproate, valproic acid, zopiclone, zotepine and ziprasidone;
(iv) anxiolytics, comprising, for example, alnespirone, azapirones,benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam and equivalents and pharmaceutically active isomer(s) and metabolite(s) thereof;
(v) anticonvulsants, comprising, for example, carbamazepine, topiramate, valproate, lamotrigine and gabapentin;
(vi) Alzheimer's therapies, comprising, for example, donepezil, memantine and tacrine;
(vii) Parkinson's therapies, comprising, for example, deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase;
(viii) migraine therapies, comprising, for example, almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan and zomitriptan;
(ix) stroke therapies, comprising, for example, abciximab, activase, (NXY-059), citicoline, crobenetine, desmoteplase,repinotan and traxoprodil;
(x) urinary incontinence therapies, comprising, for example, darifenacin, falvoxate, oxybutynin, propiverine, robalzotan, solifenacin, trypium and tolterodine;
(xi) neuropathic pain therapies, comprising, for example, gabapentin, lidoderm and pregablin;
(xii) nociceptive pain therapies, comprising, for example, celecoxib, etoricoxib, lumiracoxib, rofecoxib, valdecoxib, diclofenac, loxoprofen, naproxen and paracetamol; and
(xiii) insomnia therapies, comprising, for example, allobarbital, alonimid, amobarbital, benzoctamine, butabarbital, capuride, chloral, cloperidone, clorethate, dexclamol, eszopiclone, ethchlorvynol, etomidate, glutethimide, halazepam, hydroxyzine, mecloqualone, melatonin, mephobarbital, methaqualone, midaflur, nisobamate, pentobarbital, phenobarbital, propofol, roletamide, triclofos3secobarbital, zaleplon and Zolpidem.

General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x±10%.

General Methods of Preparation

In general, compounds of formula (I) may be prepared according to reaction schemes 1-14 (FIGS. 1-10). Suitable reaction conditions are described below.

General Procedure for Goldberg Reaction

This protocol was performed according to conditions disclosed in Org. Lett. 2003, 5 (7), 963.

To a suspension of copper(I) iodide (0.354 g, 1.86 mmol), potassium carbonate (7.35 g, 53.2 mmol) and (±)-trans-1,2-diaminocyclohexane (0.328 ml, 2.67 mmol) in dioxane, (15 ml) 9 (5 g, 26.7 mmol) and 10b (2.352 g, 27.01 mmol) were added and the reaction mixture was stirred at 100° C. for 20 h. Reaction mixture was cooled and filtered through silica gel pad with the help of EtOAc (150 ml). Filtrate was concentrated in vacuo to 11b 4.7 g (91%)

Analogous coupling reactions performed according to the above procedure and utilising the appropriate coupling partners according to the schemes gave the following yields: 11a (81%), 11c (40%), 11d (55%), 24a (74%), 24b (64%)

*The above reaction also works under the microwave conditions at 110° C. in 4 h to give 11 (50-90%).

Specific Procedure for Goldberg Reaction

Prepared according to the method of P. B. Kapadnis, PhD Thesis, University of Cambridge, 2009

The aryl bromide (1 eq), cyclic coupling partner (1.1 eq), freshly recrystallised copper (I) iodide (10 mol %), K₂CO₃ (2 eq) and (1R, 2R)-(−)-diaminocyclohexane (1 eq) were combined in anhydrous 1,4-dioxane and refluxed for 21-24 hours. The reaction mixture was then allowed to cool to r.t., concentrated in vacuo and the residue then purified to afford the desired coupling product.

79 was prepared using 76 (207 mg, 0.70 mmol), 77 (125 mg, 0.77 mmol), freshly recrystallised copper (I) iodide (14 mg, 0.07 mmol), K₂CO₃ (192 mg, 1.39 mmol), (1R, 2R)-(−)-diaminocyclohexane (80 mg, 0.70 mmol) and dioxane (7.5 mL) for 21 hours. The crude compound was suspended in a small volume of MeOH and applied to a Biotagelsolute® SCX-2 column. This was then eluted with MeOH (approx. 2 column volumes) and then 2M NH₃ in MeOH (approx. 2 column volumes). The fractions resulting from the NH₃ in MeOH elution were combined, concentrated in vacuo and suspended in boiling EtOAc until no further solid would dissolve. The hot suspension was then filtered, the solid discarded and the supernatant concentrated in vacuo. The residue was purified by flash column chromatography (SiO₂, 10% MeOH in CHCl₃) to afford 79 as a yellow amorphous solid (250 mg, 0.66 mmol, 94%).

80 was prepared using 76 (83 mg, 0.28 mmol), 78 (50 mg, 0.31 mmol), freshly recrystallised copper (I) iodide (5 mg, 0.03 mmol), K₂CO₃ (77 mg, 0.56 mmol), (1R, 2R)-(−)-diaminocyclohexane (32 mg, 0.28 mmol) and dioxane (3 mL) for 24 hours. The crude compound was partially purified by flash column chromatography (SiO₂, 10% MeOH in CHCl₃). This was then suspended in boiling EtOAc until no further solid would dissolve and then the boiling suspension filtered. The supernatant was concentrated in vacuo to afford 80 as a yellow amorphous solid (72 mg, 0.19 mmol, 68%).

General Procedure for Aryl Bromination

To a solution of 11a (3 g, 15.69 mmol) in acetic acid (30 ml), bromine (0.970 ml, 18.83 mmol) was added dropwise at rt. After stirring at rt. for 16 h reaction mixture was poured in ice-water. Precipitated compound was filtered, washed with water and dried to 12a, 2.9 g (69%) Yields: 12b (81%), 25a (93%), 25b (82%) 37 (92%).

General Procedure for Buchwald-Hartwig Coupling

In a oven dried 20 mL round-bottomed flask 2,2′-bis(diphenylphosphino)-1,1′-binaphthyl (racemic) (0.07 g, 0.112 mmol) and palladium(II) acetate (0.02 g, 0.089 mmol) in dry toluene (3 ml) were added under N₂ atm. followed by addition of (2R,6S)-2,6-dimethylpiperazine (0.101 g, 0.888 mmol), 12a (0.2 g, 0.740 mmol) and cesium carbonate (0.338 g, 1.037 mmol). Reaction mixture was stirred at 100° C. for 16 h. After 16 h reaction mixture was cooled, diluted with EtOAc and filtered through celite pad, filtrate was concentrated and purified by column chromatography with a silica gel column and was eluted with 15% MeOH in CHCl₃ to obtain a pure product 13a, 0.1 g (45%)

Analogous coupling reactions performed according to the above procedure and utilising the appropriate piperazine/piperidine and bromide coupling partners according to the schemes gave the following yields: 13b (28%), 13c (22%), 13d (37%), 13e (24%), 13f (28%), 15a (44%), 15b (40%), 22 (71%), 26a (69%), 26b (87%), 26c (38%), 30 (97%), 33a (77%), 33b (87%), 33c (36%), 39 (64%), 45 (67%), 47a (84%), 47b (83%).

General Procedure for NO₂ Reduction

To a solution of compound 3a (0.8 g, 3.18 mmol) in MeOH (10 mL) Palladium 10% on carbon (0.339 g) was added and reaction mixture was stirred under an atmosphere of hydrogen (balloon) for 1 h. The resulting mixture was filtered through a plug of Celite and the filtrate was concentrated in vacuo to give 4a, 0.4 g (57%).

Yields: 16a (50%), 16b (57%).

General Procedure for DDQ Aromatization

To a stirred solution of 25 (0.1 g, 0.354 mmol) in dioxane (10 mL) added DDQ (0.121 g, 0.532 mmol) in portions. Reaction mixture was refluxed for 16 h, cooled and filtered. Filtrate was concentrated and purified by column chromatography using silica-gel column (40% EtOAc in Hexane) to obtain 44 0.082 g (83%).

Yield: 46 (81%)

General Procedure to Prepare Sultam Derivatives.

To a solution of 16a (0.1 g, 0.423 mmol) in of CH₂Cl₂ (2 mL), triethylamine (0.18 mL, 1.28 mmol) and 3-chloropropane-1-sulfonyl chloride (57 μL, 0.467 mmol) of was added. The mixture was stirred overnight at room temperature, washed with 1 N HCl, and evaporated to dryness. The resulting crude compound 17a was dissolved in 1 mL of DMF, and DBU (65 μL, 0.423 mmol) of was added. Reaction mixture was stirred for 4 h at it, then added in water and extracted with EtOAc. The organic layer was dried over MgSO₄ and evaporated to give crude product. Crude product was loaded on silica-gel column and was eluted with 30% MeOH in CHCl3 to 18a (35 mg, 24% overall yield).

yield 18b (32%) 32 (97%).

General Procedure to Prepare 1,1-dioxo-1,2,5-thiadiazolidine Derivatives.

In a microwave tube, compound 48a (0.25 g, 0.907, 1 eq) was taken in dry THF (5 mL), followed by addition of Burgess Reagent (2.2 eq). Reaction mixture was heated at 80° C. under microwave radiations for 17 min. Reaction mixture was cooled, added to water and extracted with EtOAc; organic layer was dried and evaporated to give analytically pure 49a (70%).

General Procedure for the Synthesis of Hydrochloride Salts

To a stirring solution of the amine (1 eq) in DCM, under nitrogen, was added HCl in Et₂O (2M, 10 eq). Further DCM was added and the resulting precipitate collected, washed with a small volume of Et₂O, and dried to afford the desired hydrochloride salt.

81 was prepared using 79 (50 mg, 0.13 mmol), DCM (2 mL) and HCl in Et₂O (0.65 mL, 1.30 mmol), to afford 81 as a yellow amorphous solid (31 mg).

82 was prepared using 80 (20 mg, 0.053 mmol), DCM (1 mL) and HCl in Et₂O (0.27 mL, 0.53 mmol) to afford 82 as a yellow amorphous solid (10 mg).

Preparation of 2

This protocol was performed according to conditions disclosed in J. Org. Chem. 1993, 58 (19), 5101.

In a 50 mL round-bottomed flask n-methyl piperazine (0.926 g, 9.25 mmol, 1 eq.) was taken in THF (14 ml). At 0° C. n-Butyllithium 1.6M hexanes (0.940 ml, 10.17 mmol) was added dropwise. Reaction mixture was stirred at 0° C. for 30 min and at rt. for 1 h. Veratrole (1.1 eq) was added to the reaction mixture and reaction mixture was refluxed for 16 h. Reaction mixture was cooled and poured into cold 2N HCl solution followed by extraction with EtOAc. Aqueous layer was collected, basified and extracted with EtOAc. Organic layer was dried (MgSO₄), filtered and concentrated to oily product 2a, 0.5 g (29%).

Yield 2b (45%).

Preparation of 3

In a 10 mL round-bottomed flask, compound 2 (0.1 g, 0.485 mmol) was taken in 5N H₂SO₄ solution (0.1 mL) and the resulting was concentrated to dryness in vacuo. Sulfuric acid (0.67 ml, 12.57 mmol) was added and the mixture was stirred for 10 min. Reaction mixture was cooled to 0° C. and KNO₃ (0.11 g, 1.035 mmol) was added portion-wise maintaining the temperature below 10° C. Reaction mixture was then allowed to warm at rt and then stirred at rt. for 16 h. Reaction mixture was poured on to ice water, neutralized by addition of Na₂CO₃ and extracted by EtOAc. Organic layer was dried (MgSO₄), filtered and concentrated to 3a 97 mg (80%).

Yield 3b (81%).

Preparation of 5

This protocol was performed according to conditions disclosed in Tetrahedron 2001, 57 (47), 9635.

To a suspension of disodium phosphate (0.257 g, 1.808 mmol) in chloroform (4 ml) compound 4a (0.2 g, 0.904 mmol) was added and stirred followed by dropwise addition of 4-bromobutanoyl chloride (0.105 ml, 0.904 mmol) at rt. Reaction mixture was stirred at rt. for 16 h and filter through Celite plug, filtrate was concentrated in vacuo and directly used for next step. The crude product was added to a solution of sodium methoxide (0.090 ml, 2.166 mmol) in MeOH (2 ml) and the resulting mixture was stirred at rt. for 16 h. Solvents were evaporated and the crude product was added to a silica gel column and was eluted with 15% MeOH in CHCl₃ to obtain pure product 5a, 0.11 g (42.1%).

Preparation of 6

This protocol was performed according to conditions disclosed in Synthesis 2002, 2, 221.

To a solution of compound 4a (0.1 g, 0.452 mmol) in dioxane (1 ml) and toluene (2 ml), succinic anhydride (0.045 g, 0.452 mmol) in Et20 was added dropwise over a period of 20 min at rt. Reaction mixture was stirred at rt. for 2 h. The precipitated solid was then filtered through Buchner funnel, washed with Et₂O and vacuum dried to the product 6, 0.11 g (76%).

Preparation of 7

To a solution of compound 6 (0.1 g, 0.311 mmol) in Acetic anhydride (0.25 ml), Sodium acetate (0.01 g, 0.122 mmol) was added. Resulting solution was heated to 60° C. for 2 h. The mixture was cooled to r.t. and poured into ice-cold water. Precipitated solid was filter, washed with water, dried to provide 7, 50 mg (53%).

Preparation of 20

To a solution of pyrrolidin-2-one (1 g, 11.75 mmol) in toluene added K₂CO₃ containing 16 wt. % water (3.25 g, 23.5 mmol), TBAB (0.38 g, 1.17 mmol) and 4-methoxybenzylchloride (1.84 g, 11.75 mmol). Reaction mixture was stirred at 80° C. After 24 h reaction mixture was cooled, filtered and evaporated. Crude product was purified by column chromatography. Crude product was loaded on silica-gel column and was eluted with 30% EtOAc in Hexane to provide 20 (1 g, 42%).

Preparation of 35

A solution of Boc-Tyr 34 (0.5 g, 101 mmol) in THF (337 mL) at 0° C. was treated with 1M BH₃. THF complex (4.3 mL) for 30min. The ice bath was removed and the solution was stirred at room temperature for 3 h. The reaction was cooled to 0° C. and quenched slowly with the dropwise addition of brine. The layers were separated, and the aqueous layer was extracted twice with EtOAc. The combined organic layers were dried (MgSO₄), filtered and concentrated to provide 35 (82%).

Preparation of 36

To a suspension of sodium hydride (1.45 g, 36.4 mmol) in THF (20 mL) was added a solution of 35 (0.4 g, 1.42 mmol) in THF (10 mL) over a period of 10 min. Reaction mixture was then refluxed for 3h, cooled and slowly quenched with a saturated solution of aqueous ammonium chloride followed by extraction with EtOAc. The organic layers combined, washed with aqueous hydrochloric acid, dried over magnesium sulfate and evaporated to 36 (0.292g, 99%).

Preparation of 48a

In a microwave tube compound 47a (0.5 g, 1.659 mmol) was taken in aq.10% NaOH solution. Reaction mixture was heated under microwave radiations at 100° C. for 20min (Caution! Controlled heating needed). Reaction mixture was cooled and extracted with EtOAc; organic layer was dried and evaporated. Crude compound was purified by column chromatography using a silica-gel column (20% MeOH in CH₂Cl₂) to obtain 48a, 0.3 g (66%).

Preparation of 50a

This protocol was performed according to conditions disclosed in Chem. Eur. 1 2004, 10(22), 5581.

10% aqueous NaOH (0.5 mL) was added to a solution of 49a (0.15 g, 0.38 mmol, 1.0 equiv) in MeOH/H₂O (2:1, 6 mL) at rt. After stirring this mixture for 2 h at ft., the reaction mixture was poured into saturated aqueous NH₄Cl (10 mL) and extracted with EtOAc. The combined organic layers were then dried (MgSO₄) and concentrated to give 50a (86%).

Preparation of 52

A suspension 51 (0.1 g, 0.43 mmol) in unstabilized 57% Hl (1.3 mL) was heated at 90° C. for 5 h. Reaction mixture was cooled, diluted with EtOAc (5 mL) and washed with saturated aq Na₂S₂O₃ and brine. The organic layer was dried over anhydrous MgSO₄, filtered and concentrated. The crude product was further purified by silica-gel column chromatography to 52 (0.07 g, 80%).

Preparation of 53

To a solution of 52 (0.08 g, 0.395 mmol) in CH₂Cl₂ (3 mL), added pyridine (74 μL) and methyl 3-(chlorosulfonyl)propanoate (0.1 g, 0.544 mmol). Reaction mixture was stirred for 16 h at rt. and then poured in 10% HCl solution followed by extraction with CH₂Cl₂. Organic layer was dried and evaporated to obtained 53 (52mg, 34%).

Preparation of 54

To a suspension of 53 (50 mg, 0.142 mmol) in water (1 mL) added a solution of KOH (25 mg, 0.426 mmol) in water (1 mL), reaction mixture was stirred at rt. for 1 h and then acidified with dilute HCl. Precipitated product was filtered and dried to 54 (25 mg, 52%).

Preparation of 55

Compound 54 (25 mg, 0.074 mmol) was added to SOCl₂ (0.2 mL, 2.74 mmol) and the resulting mixture was stirred at 80° C. for 2 h. Reaction mixture was neutralized with saturated sodium bicarbonate solution and extracted with CH₂Cl₂ Organic layer was dried and evaporated to 55 (15 mg, 63%).

Preparation of 59

To a solution of 58 (0.09 g, 0.342 mmol) in acetonitrile (2 mL) added N,N-diisopropylethylamine followed by addition of mechlorethamine hydrochloride. Reaction mixture was refluxed for 16 h, cooled and poured into water followed by extraction with EtOAc. Organic layer was dried and evaporated. Crude product was purified by column chromatography by using silica-gel column (10% MeOH in CH₂Cl₂) to obtain 59, 0.065 g (55%).

Preparation of 71

Prepared according to the method of Wishka et al, WO 2002/100857.

Bromine (21.6 mL, 421 mmol) was added dropwise to a stirred solution of sodium hydroxide (39.2 g, 976 mmol)) in water (800 mL) at 0° C. The resultant bromate solution was then added dropwise to a stirred solution of 3-hydroxypyridine (20.0 g, 210 mmol), and sodium hydroxide (8.4 g, 34.3 mmol) in water (50 mL) at 0° C. The reaction mixture was stirred at 0° C. for 90 minutes, acidified to pH 2 by addition of 12M HCl soln., and the resultant precipitate collected, washed with water and dried on the filter. The solid was dissolved in EtOAc (170 mL), the solution diluted with heptane (620 mL) and allowed to crystallise for 3 days. The solid was collected, to give 2-bromopyridin-3-ol, and the mother liquor concentrated in vacuo to give a pale yellow solid. The crude solid was recrystallised from EtOH/water and dried in vacuo to afford 71 as a pale yellow crystalline solid (10.8 g, 42.7 mmol, 20%).

Preparation of 72

Adapted from the method of Wishka et al, WO 2002/100857.

71 (10.0 g, 39.5 mmol), sodium bicarbonate (12.0 g, 142.8 mmol) and iodine (12.4 g, 48.9 mmol) were combined in water (200 mL) and stirred at r.t. for 5 days. Excess iodine was then quenched by addition of sodium thiosulfate (12.0 g) and the pH was adjusted to 2 by addition of conc. HCl. The resultant precipitate was collected and purified twice by flash column chromatography (SiO₂, gradient elution from 100% pet. ether to 100% EtOAc), ground to a fine powder and dried in vacuo to afford 72 as a pale pink amorphous solid ([98% purity by 1H NMR spectroscopy, where the remaining impurity was 71.Used without further purification.]14.0 g, 36.6 mmol, 92%).

Preparation of 73

Prepared according to the method of Walker et al. WO 2003/029252.

72 (98% purity, 10.0 g, 26.04 mmol), PdCl₂(PPh₃)₂ (555 mg, 0.79 mmol, 3.0 mol %), copper (I) iodide (75 mg, 0.39 mmol, 1.5 mol %) and trimethylsilylacetylene (2.64 mL, 37.84 mmol) were dissolved in CHCl₃ (43 mL) and THF (23 mL) under nitrogen. Triethylamine (11.2 mL, 80.22 mmol) was added, the reaction mixture stirred for 3 hours and then diluted with CHCl₃ (100 mL). This was then washed with 5% HCl soln. (2×100 mL), and the combined aqueous washings were then extracted with CHCl₃ (2×35 mL). All combined organic fractions were dried (MgSO₄), filtered through a pad of Celite® and concentrated in vacuo. The residue was then purified four times by flash column chromatography [SiO₂, a) 35% EtOAc in pet.ether, b) gradient elution from 100% pet. ether to 35% EtOAc in pet. ether, c) 20% EtOAc in pet. ether, d) gradient elution from 100% pet. ether to 100% EtOAc] to afford 73 as a pale yellow amorphous solid (3.8 g, 10.88 mmol, 42%).

Preparation of 74 and 75

Prepared according to the method of Walker et al. WO 2003/029252.

73 (0.44 g, 1.26 mmol), copper (I) iodide (12 mg, 0.06 mmol, 4.8 mol %) and triethylamine (2.5 mL, 17.94 mmol) were combined in ethanol (2.5 mL) and heated to 70° C. for 3.5 hours. The reaction mixture was then allowed to cool to r.t., concentrated in vacuo and partitioned between 5% HCl soln.

(10 mL) and DCM (5 mL). The aqueous layer was then further extracted with DCM (3×5 mL). The combined organic extracts were dried (MgSO₄) and concentrated in vacuo. The residue was then purified by flash column chromatography (SiO₂, 25% EtOAc in pet. ether) to afford 74 as a pale brown amorphous solid (29 mg, 0.10 mmol, 8%) and 75 as a pale brown amorphous solid (209 mg,0.60 mmol, 46%).

A mixture of 74 and 75 (1 : 4.4 molar ratio, respectively, 2.70 g, 1.48 mmol 74: 6.55 mmol 75 was dissolved in THF (60 mL) under nitrogen. TBAF (7.9 mL, 1M in THF) was added in one portion and the reaction mixture stirred at r.t. for 2.5 hours. The reaction mixture was then diluted with EtOAc (500 mL) and washed with 1M HCl soln. (2×250 mL), dried (MgSO₄) and evaporated to give a brown solid. The solid was suspended in boiling EtOAc until no further solid would dissolve, and the hot suspension filtered, the insoluble solid discarded, and the supernatant concentrated in vacuo to afford 74 as a pale brown amorphous solid (1.99 g, 7.17 mmol, 87% [yield calculated for second step of the reaction, taking into account initial presence of 74 in mixture]).

Preparation of 76

Method adapted from Tran et al. J. Med. Chem., 2007, 50, 6356-6366.

74 (1.00 g, 3.61 mmol), 1-methylpiperazine (0.36 mL, 3.25 mmol), DIPEA (0.57 mL, 3.27 mmol), and DMF (50 mL) were combined and heated to 100° C. under nitrogen for 5 hours. The reaction mixture was allowed to cool to r.t., poured into sat. NaHCO₃ solution (50 mL) and extracted with EtOAc (4×50 mL), then CHCl₃ (2×50 mL). The combined organic extracts were dried (Na₂SO₄) and concentrated in vacuo. The residue was then purified by flash column chromatography (SiO₂, 3% MeOH in DCM) to afford 76 as a yellow oil (0.64 g, 2.16 mmol, 60%).

Preparation of 77

2-Amino-1-phenylethanol (2.74 g, 20.0 mmol) and CDI (3.31 g, 20.4 mmol) were combined in DCM (25 mL) and stirred at r.t. under nitrogen for 2 hours. The reaction mixture was poured into EtOAc (100 mL) and washed with water (2×50 mL). On standing, precipitate formed in the organic layer, which was filtered off and discarded. The mother liquor was then dried (MgSO₄), concentrated in vacuo and the residue purified by flash column chromatography (SiO₂, gradient elution from 2% MeOH in EtOAc to 5% MeOH in EtOAc) to afford 77 as a white amorphous solid (1.52 g, 9.32 mmol, 47%).

Preparation of 78

Prepared according to the method of Samuel and Santini WO 2007/070433.

To a stirred solution of 2-chloroethylamine hydrochloride (270 mg, 2.33 mmol) in anhydrous DMF (3 mL) was added phenyl isocyanate (254 μL, 2.33 mmol), then Cs₂CO₃ (758 mg, 2.33 mmol). The vessel was then flushed with nitrogen and the mixture stirred under nitrogen for 6 hours. Potassium tert-butoxide (261 mg, 2.33 mmol) was then added, the vessel flushed with nitrogen and the mixture stirred under nitrogen overnight. Water was added and the resultant precipitate collected and partially purified by flash column chromatography (SiO₂, 2.5% MeOH in CHCl₃). The mixture was then suspended in DCM (5 mL), the insoluble solid filtered off, washed with a little DCM and discarded, and the supernatant concentrated in vacuo. This suspension-filtration-concentration process was repeated a further two times to afford 78 as a pale yellow amorphous solid ([86% purity by 1H NMR spectroscopy. Used in subsequent reactions without further purification] 67 mg, 0.35 mmol, 15%).

Preparation of 83

A suspension of 76 (92 mg, 0.311 mmol), 2-pyrrolidinone (0.03 mL, 0.342 mmol), copper (I) iodide (0.04 g), (1R, 2R)-(-)-diaminocyclohexane (50 mg) and K₂CO₃ (0.09 g, 0.622 mmol) in anhydrous dioxane (3 mL) under nitrogen was heated at 115° C. for 24 hours. Extra 2 pyrrolidinone (0.02 mL) was added and the mixture heated at 130° C. for a further 19 hours. TLC analysis indicated complete consumption of starting material. The mixture was cooled to room temperature, filtered through a pad of Celite® washing with EtOAc followed by CHCl₃:MeOH 1:1 volume/volume mix) and concentrated in vacuo. The crude product material was purified by column chromatography (SiO₂, gradient elution 4% MeOH in CHCl₃ to 8% MeOH inCHCl₃) to yield an off-white solid which spectroscopic analysis indicated was a mixture of 83 and unreacted 2 pyrrolidinone. The crude compound was suspended in a small volume of MeOH and applied to a Biotagelsolute® SCX-2 column. This was then eluted with MeOH (approx. 2 column volumes) and then 2M NH₃ in MeOH (approx. 2 column volumes). The fractions resulting from the NH₃ in MeOH elution were combined and concentrated in vacuo to yield 83 as an off-white solid (79 mg, 0.28 mmol, 89%).

Preparation of 84

A suspension of 76 (113 mg, 0.382 mmol), 2-oxazolidone (0.068 g, 0.342 mmol), copper (I) iodide (0.05 g), (1R, 2R)-(-)-diaminocyclohexane (50 mg) and K₂CO₃ (0.11 g, 0.76 mmol) in anhydrous dioxane (7 mL) under nitrogen was heated at 100° C. for 21 hours. Extra 2-oxazolidone (0.02 g) and copper (I) iodide (0.05 g) was added and the mixture heated at 115° C. for a further 24 hours. Additional 2-oxazolidone (0.07 g), copper (I) iodide (0.05 g), potassium carbonate (0/11 g) and (1R, 2R)-(−)-diaminocyclohexane (50 mg) was added and the reaction heated at 125° C. for a further 24 hours. TLC analysis indicated complete consumption of starting material. The mixture was cooled to room temperature, filtered through a pad of Celite® washing with CHCl₃:MeOH (1:1 volume/volume mix) and concentrated in vacuo. The crude compound was suspended in a small volume of MeOH and applied to a BiotageIsolutee SCX-2 column. This was then eluted with MeOH (approx. 2 column volumes) and then 2M NH₃ in MeOH (approx. 2 column volumes). The fractions resulting from the NH₃ in MeOH elution were combined and concentrated in vacuo yield a brown film. Purification by column chromatography (SiO₂, gradient elution 3% MeOH in CHCl₃ to 6% MeOH in CHCl₃) yielded 84 as an off-white solid (79 mg, 0.262 mmol, 68%).

Table 1 provides characterization data for intermediates prepared according to the above methods.

TABLE 1

| Compound | | | (M + H)⁺ or |
|---|---|---|---|
| No. | Structure | ¹H & ¹³C NMR | (M + Na)⁺ |
| 2b | 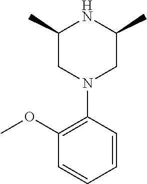 | ¹H NMR (400 MHz, CDCl3) δ 7.10-6.70 (m, 4H), 3.83 (s, 3H), 3.34 (d, J = 11.2, 2H), 3.24-2.99 (m, 2H), 2.19 (t, J = 10.3, 2H), 1.77 (s, 1H), 1.23-0.95 (d, 6H). ¹³C NMR (101 MHz, CDCl3) δ 152.92, 142.00, 123.44, 121.60, 119.08, 111.91, 58.56, 56.00, 51.41, 20.41. | found 221.1650 calculated 221.1654 C₁₃H₂₁N₂O |
| 3a | 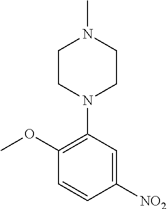 [SMILES: COc1ccc([N+](=O)[O-])cc1N1CCN(C)CC1] | ¹H NMR (400 MHz, CDCl3) δ 7.93 (dd, J = 2.7, 8.9, 1H), 7.78 (d, J = 2.7, 1H), 6.88 (d, J = 9.0, 1H), 3.96 (s, 3H), 3.14 (s, 4H), 2.62 (s, 4H), 2.36 (s, 3H). ¹³C NMR (101 MHz, CDCl3) δ 157.66, 141.96, 119.39, 113.89, 110.43, 110.34, 76.91, 56.40, 55.33, 50.59, 46.36. | Found 252.1350 Calculated 252.1348 C₁₂H₁₈N₃O₃ |
| 3b | 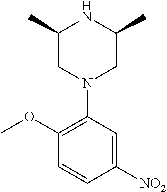 [SMILES: COc1ccc([N+](=O)[O-])cc1N1C[C@@H](C)N[C@@H](C)C1] | ¹H NMR (400 MHz, CDCl3) δ 7.90 (dd, J = 2.7, 9.0, 1H), 7.74 (d, J = 2.7, 1H), 6.86 (d, J = 9.0, 1H), 3.94 (s, 3H), 3.36 (d, J = 9.8, 2H), 3.21-3.00 (m, 2H), 2.23 (t, J = 10.8, 2H), 1.75 (s, 3H), 1.11 (d, J = 6.4, 7H). ¹³C NMR (101 MHz, CDCl3) δ 171.44, 157.62, 141.81, 141.59, 119.36, 113.89, 110.39, 60.53, 57.27, 56.29, 50.70, 21.04, 19.38, 14.19. | found 266.1496 calculated 266.1505 C₁₃H₂₀N₃O₃ |
| 4a | 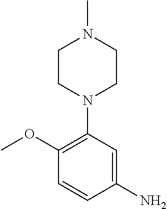 [SMILES: COc1ccc(N)cc1N1CCN(C)CC1] | ¹H NMR (400 MHz, MeOH) δ 6.65 (d, J = 8.3, 1H), 6.30 (dt, J = 2.7, 8.3, 2H), 3.76 (s, 3H), 3.07 (s, 5H), 2.61 (s, 5H), 2.33 (d, J = 5.9, 3H). ¹³C NMR (101 MHz, CDCl3) δ 146.13, 142.77, 140.98, 113.58, 109.37, 107.48, 56.54, 55.91, 50.95, 46.63. | Found 222.1608 Calculated 222.1606 C₁₂H₂₀N₃O |
| 11a | 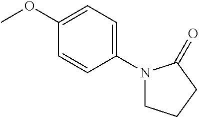 | ¹H NMR (400 MHz, CDCl3) δ 7.47 (d, J = 9.2, 2H), 6.88 (d, J = 9.1, 2H), 3.80 (t, J = 7.0, 2H), 3.78 (s, 3H), 2.57 (t, J = 8.1, 2H), 2.24-2.03 (m, 2H). ¹³C NMR (101 MHz, CDCl3) δ 174.14, 156.77, 132.81, 122.06, 114.24, 55.68, 49.42. 32.68, 18.26. | Found 192.1022 Calculated 192.1025 C₁₁H₁₄NO₂ |
| 11b | 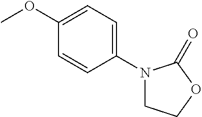 | ¹H NMR (400 MHz, CDCl3) δ 7.46-7.35 (m, 2H), 6.94-6.84 (m, 2H), 4.57-4.31 (m, 2H), 4.00 (dd, J = 7.2, 8.8, 2H), 3.78 (s, 3H). ¹³C NMR (101 MHz, CDCl3) δ 156.58, 155.79, 131.63, 120.49, 114.48, 61.47, 55.71, 45.92, | Found 194.0822 Calculated 194.0817 C₁₀H₁₂NO₃ |
| 12a | 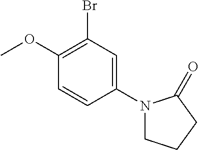 | ¹H NMR (400 MHz, CDCl3) δ 7.73 (d, J = 2.6, 1H), 7.53 (dd, J = 2.6, 8.9, 1H), 6.85 (d, J = 9.0, 1H), 3.84 (s, 3H), 3.76 (t, J = 7.0, 2H), 2.55 (t, J = 8.1, 2H), 2.20- 2.06 (m, 2H). ¹³C NMR (101 MHz, CDCl3) δ 174.17, 152.93, 133.51, 125.24, 120.60, 111.90, 111.52, 56.59, 49.09, 32.59, 18.04. | Found 270.0129 Calculated 270.0130 C₁₁H₁₃NO₂Br |
| 12b | 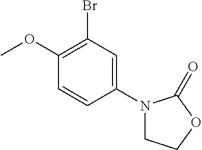 | ¹H NMR (400 MHz, CDCl3) δ 7.66 (d, J = 2.8, 1H), 7.43 (dd, J = 2.8, 9.0, 1H), 6.85 (d, J = 9.0, 1H), 4.43 (dd, J = 7.2, 8.8, 2H), 3.97 (dd, J = 7.2, 8.8, 2H), 3.84 (s, 3H). ¹³C NMR (101 MHz, CDCl3) δ 155.41, 152.76, 132.33, 123.72, 118.93, 112.16, 111.83, 61.46, 56.64. | Found 271.9923 Calculated 271.9925 C₁₀H₁₁NO₃Br |
| 20 | 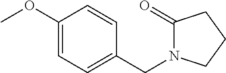 | ¹H NMR (400 MHz, CDCl3) δ 7.26 (d, J = 8.5, 2H), 7.11 (d, J = 8.6, 2H), 4.34 (s, 2H), 3.83 (s, 3H), 3.21 (app t, 2H), 2.41 (t, J = 8.1, 2H), 1.98-1.89 (m, 2H). ¹³C NMR (101 MHz, CDCl3) δ 175.31, 174.77, 219.46, 128.39, 114.02, 55.22, 46.60, 46.03, 30.94, 17.60. | found 206.1185 calculated 206.1181 C₁₂H₁₆NO₂ |
| 21 | 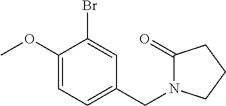 [SMILES: COc1ccc(CN2CCCC2=O)cc1Br] | ¹H NMR (400 MHz, CDCl3) δ 7.37 (d, J = 2.1, 1H), 7.11 (dd, J = 2.0, 8.4, 1H), 6.80 (d, J = 8.4, 1H), 4.31 (s, 2H), 3.83 (s, 3H), 3.26-3.15 (m, 2H), 2.38 (t, J = 8.1, 2H), 1.98-1.87 (m, 2H). ¹³C NMR (101 MHz, CDCl3) δ 174.98, 155.46, 133.13, 130.40, 128.54, 112.14, 111.87, 56.42, 46.64, 45.59, 31.01, 17.83. | found 284.0286 calculated 340.1695 C₁₂H₁₅BrNO₂ |
| 24a | 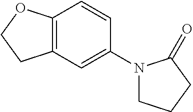 [SMILES: O=C1CCCN1c1ccc2c(c1)CCO2] | ¹H NMR (400 MHz, CDCl3) δ 7.47 (s, 1H), 7.08 (d, J = 8.6, 1H), 6.70 (d, J = 8.6, 1H), 4.50 (t, J = 8.7, 2H), 3.75 (t, J = 7.0, 2H), 3.16 (t, J = 8.6, 2H), 2.52 (t, J = 8.0, 2H), 2.16-2.02 (m, 2H). ¹³C NMR (101 MHz, CDCl3) δ 173.98, 157.35, 132.53, 127.65, 120.64, 118.75, 109.01, 71.55, 49.78, 32.49, 30.02, 18.17. | found 204.1027 calculated 204.1025 C₁₂H₁₄NO₂ |
| 24b | 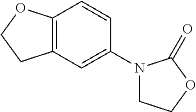 [SMILES: O=C1OCCN1c1ccc2c(c1)CCO2] | ¹H NMR (400 MHz, CDCl3) δ 7.53-7.45 (d, J = 2.5, 1H), 7.03 (dd, J = 2.5, 8.6, 1H), 6.74 (d, J = 8.6, 1H), 4.55 (t, J = 8.7, 2H), 4.43 (dd, J = 7.2, 8.8, 2H), 3.99 (dd, J = 7.2, 8.8, 2H), 3.20 (t, J = 8.7, 2H). ¹³C NMR (101 MHz, CDCl3) δ 157.31, 155.98, 131.47, 128.23, 119.23, 117.40, 109.28, 71.73, 61.49, 46.40, 30.15. | found 206.0814 calculated 206.0817 C₁₁H₁₂NO₃ |
| 25a | 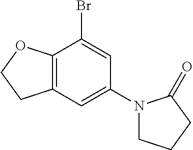 [SMILES: O=C1CCCN1c1cc(Br)c2c(c1)CCO2] | ¹H NMR (400 MHz, CDCl3) δ 7.52 (s, 1H), 7.29 (s, 1H), 4.63 (t, J = 8.7, 2H), 3.76 (t, J = 7.0, 2H), 3.29 (t, J = 8.7, 2H), 2.55 (t, J = 8.1, 2H), 2.20-2.05 (m, 2H). ¹³C NMR (101 MHz, CDCl3) δ 174.18, 154.71, 133.71, 128.72, 123.13, 117.64, 101.96, 71.99, 49.68, 32.60, 31.11, 18.19. | found 282.0131 calculated 282.0130 C₁₂H₁₃BrNO₂ |
| 25b | 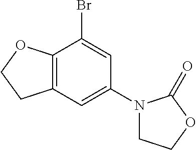 [SMILES: O=C1OCCN1c1cc(Br)c2c(c1)CCO2] | ¹H NMR (400 MHz, CDCl3) δ 7.52-7.44 (m, 1H), 7.20 (d, J = 2.2, 1H), 4.64 (t, J = 8.7, 2H), 4.43 (dd, J = 7.2, 8.8, 2H), 3.97 (dd, J = 7.2, 8.8, 2H), 3.30 (t, J = 8.7, 2H). ¹³C NMR (101 MHz, CDCl3) 155.47, 154.36, 132.30, 128.97, 121.41, 115.87, 101.89, 71.80, 61.27, 45.92, 30.88 | found 283.9923 calculated 283.9922 C₁₁H₁₁BrNO₃ |
| 29 | 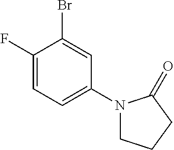 [SMILES: O=C1CCCN1c1ccc(F)c(Br)c1] | ¹H NMR (400 MHz, CDCl3) δ 7.82 (dd, J = 2.7, 6.1, 1H), 7.61- 7.47 (m, 1H), 7.08 (t, J = 8.5, 1H), 3.80 (t, J = 7.0, 2H), 2.59 (t, J = 8.1, 2H), 2.27-2.02 (m, 2H). ¹³C NMR (101 MHz, CDCl3) δ 174.35, 154.77, 136.62, 124.82, 120.49, 120.42, 116.54, 116.31, 109.21, 108.99, 48.99, 32.68, 18.04. | found 257.9932 calculated 257.9930 C₁₀H₁₀BrFNO |
| 32 | 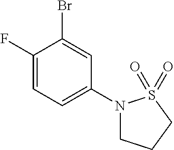 [SMILES: O=S1(=O)CCCN1c1ccc(F)c(Br)c1] | ¹H NMR (400 MHz, CDCl3) δ 7.41 (dd, J = 2.8, 5.8, 1H), 7.24- 7.17 (m, 1H), 7.10 (dd, J = 8.1, 8.8, 1H), 3.70 (t, J = 6.6, 2H), 3.43-3.28 (m, 2H), 2.58-2.44 (m, 2H). ¹³C NMR (101 MHz, CDCl3) δ 125.06, 121.00, 120.93, 116.94, 116.70, 47.89, 47.09, 18.61. | found 315.9413 calculated 315.9419 C₉H₉BrFNO₂SNa |
| 35 | 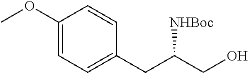 [SMILES: COc1ccc(C[C@@H](CO)NC(=O)OC(C)(C)C)cc1] | ¹H NMR (500 MHz, CDCl3) δ 7.10 (d, J = 8.5, 2H), 6.86-6.77 (m, 2H), 4.78 (s, 1H), 3.90-3.69 (m, 4H), 3.68-3.38 (m, 2H), 2.75 (d, J = 7.0, 2H), 1.39 (s, 9H). ¹³C NMR (126 MHz, CDCl3) δ 158.45, 156.40, 130.43, 129.97, 114.14, 79.87, 64.44, 55.44, 54.01, 36.69, 28.54. | found 304.1517 calculated 304.1511 C₁₃H₂₁N₄O₃Na |
| 36 | 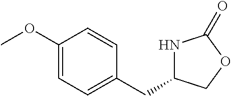 | ¹H NMR (400 MHz, CDCl3) δ 7.07 (d, J = 8.5, 2H), 6.89-6.76 (m, 2H), 5.61 (s, 1H), 4.41 (t, J = 8.3, 1H), 4.10 (dd, J = 5.7, 8.5, 1H), 4.06-3.96 (m, 1H), 3.76 (d, J = 6.3, 3H), 2.79 (d, J = 6.8, 2H). ¹³C NMR (101 MHz, CDCl3) δ 159.54, 159.00, 130.20, 128.06, 114.61, 69.79, 55.49, 54.11, 40.74. | found 208.0967 calculated 208.0974 C₁₁H₁₄NO₃ |
| 37 | 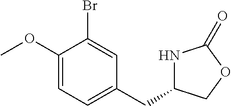 [SMILES: COc1ccc(C[C@H]2COC(=O)N2)cc1Br] | ¹H NMR (400 MHz, CDCl3) δ 7.34 (d, J = 1.8, 1H), 7.07 (d, J = 8.3, 1H), 6.84 (d, J = 8.3, 1H), 5.68 (s, 1H), 4.42 (t, J = 8.2, 1H), 4.16-3.95 (m, 2H), 3.86 (s, 3H), 2.77 (d, J = 6.6, 2H). ¹³C NMR (101 MHz, CDCl3) δ 159.48, 155.38, 133.89, 129.62, 129.29, 112.51, 112.33, 69.69, 56.51, 53.92, 40.36. | found 286.0082 calculated 286.0079 C₁₁H₁₄NO₃ |
| 44 | 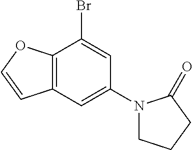 [SMILES: O=C1CCCN1c1cc(Br)c2occc2c1] | ¹H NMR (400 MHz, CDCl3) δ 7.73 (d, J = 2.0, 1H), 7.67 (d, J = 2.0, 1H), 7.64 (d, J = 2.1, 1H), 6.79 (d, J = 2.2, 1H), 3.85 (t, J = 7.0, 2H), 2.59 (t, J = 8.1, 2H), 2.23-2.08 (m, 2H). ¹³C NMR (101 MHz, CDCl3) δ 174.60, 149.65, 146.65, 135.75, 128.63, 120.74, 112.84, 107.94, 103.98, 49.90, 32.65, 18.19. | found 279.9974 calculated 279.9973 C₁₂H₁₁BrNO₂ |
| 46 | 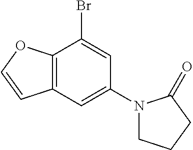 [SMILES: O=C1CCCN1c1cc(Br)c2occc2c1] | ¹H NMR (400 MHz, CDCl3) δ 7.70 (d, J = 2.1, 1H), 7.67 (d, J = 2.2, 1H), 7.65 (d, J = 2.1, 1H), 6.80 (d, J = 2.2, 1H), 4.48 (ddd, J = 2.8, 8.2, 11.0, 2H), 4.17-3.94 (m, 2H). ¹³C NMR (101 MHz, CDCl3) δ 155.62, 149.45, 146.94, 134.88, 128.89, 119.06, 110.90, 107.93, 104.36, 61.50, 46.19. | found 281.9777 calculated 281.9766 C₁₁H₉BrNO₃ |
| 48a | 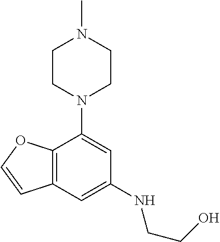 [SMILES: CN1CCN(c2cc(NCCO)cc3ccoc23)CC1] | ¹H NMR (500 MHz, CDCl3) δ 7.48 (d, J = 2.1, 1H), 6.58 (d, J = 2.1, 1H), 6.40 (d, J = 2.1, 1H), 6.13 (d, J = 2.1, 1H), 3.93-3.77 (m, 2H), 3.33 (s, 4H), 3.30-3.26 (m, 2H), 2.72-2.60 (m, 4H), 2.37 (s, 3H), 2.19 (s, 1H). ¹³C NMR (126 MHz, CDCl3) δ 145.27, 144.41, 141.54, 137.95, 129.43, 106.95, 101.06, 96.03, 61.53, 55.37, 49.78, 47.39, 46.38. | found 276.1707, calculated 276.1712 C₁₅H₂₂N₃O₂ |
| 53 | 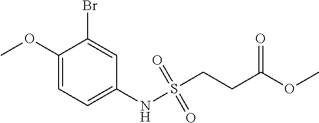 | ¹H NMR (400 MHz, CDCl3) δ 7.47 (d, J = 2.7, 1H), 7.22 (dd, J = 2.7, 8.8, 1H), 6.86 (d, J = 8.8, 1H), 6.49 (s, 1H), 3.87 (s, 3H), 3.71 (s, 3H), 3.35 (t, J = 7.2, 2H), 2.85 (t, J = 7.2, 2H). ¹³C NMR (101 MHz, CDCl3) δ 171.37, 154.76, 129.78, 128.41, 123.75, 112.52, 112.31, 56.73, 52.73, 46.92, 29.00. | found 351.9850, calculated 351.9854 C₁₁H₁₅BrNO₅S |
| 54 | 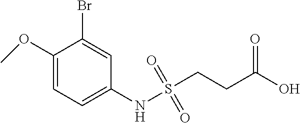 [SMILES: COc1ccc(NS(=O)(=O)CCC(=O)O)cc1Br] | ¹H NMR (400 MHz, CDCl3) δ 7.46 (d, J = 2.6, 1H), 7.21 (dd, J = 2.6, 8.8, 1H), 6.86 (d, J = 8.8, 1H), 6.39 (s, 1H), 3.87 (s, 3H), 3.36 (t, J = 7.2, 2H), 2.91 (t, J = 7.2, 2H). ¹³C NMR (126 MHz, CDCl3) δ 172.34, 154.88, 129.55, 128.55, 123.85, 112.56, 112.38, 56.75, 46.82, 28.36 | found 359.9519 calculated 359.9517 C₁₀H₁₂BrNO₅SNa |
| 55 | 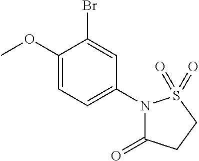 [SMILES: COc1ccc(N2C(=O)CCS2(=O)=O)cc1Br] | ¹H NMR (400 MHz, CDCl3) δ 7.55 (d, J = 2.5, 1H), 7.29 (dd, J = 2.5, 8.8, 1H), 6.97 (d, J = 8.8, 1H), 3.91 (s, 3H), 3.81-3.68 (m, 2H), 3.28-3.16 (m, 2H). ¹³C NMR (101 MHz, CDCl3) δ 165.54, 157.55, 133.67, 129.17, 121.97, 112.66, 112.38, 56.76, 47.08, 29.94. | |
| 57 | 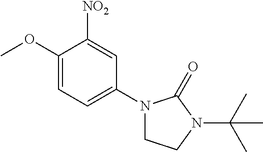 [SMILES: COc1ccc(N2CCN(C(C)(C)C)C2=O)cc1[N+](=O)[O-]] | ¹H NMR (500 MHz, CDCl3) δ 7.94 (dd, J = 2.9, 9.2, 1H), 7.82 (d, J = 2.9, 1H), 7.02 (d, J = 9.2, 1H), 3.91 (s, 3H), 3.68 (dd, J = 6.6, 8.9, 2H), 3.50 (dd, J = 6.6, 9.1, 2H), 1.40 (s, 9H). ¹³C NMR (126 MHz, CDCl3) δ 157.83, 148.37, 139.40, 134.35, 124.10, 114.33, 114.09, 57.07, 53.96, 42.33, 40.09, 27.68. | found 294.1458, calculated 294.1454 C₁₄H₂₀N₃O₄ |
| 58 | 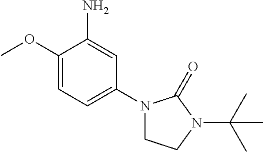 [SMILES: COc1ccc(N2CCN(C(C)(C)C)C2=O)cc1N] | ¹H NMR (500 MHz, CDCl3) δ 7.18 (d, J = 2.5, 1H), 6.69 (d, J = 8.7, 1H), 6.58 (dd, J = 2.6, 8.7, 1H), 3.79 (s, 3H), 3.65-3.56 (m, 2H), 3.46-3.36 (m, 2H), 1.38 (s, 9H). ¹³C NMR (126 MHz, CDCl3) δ 158.63, 143.42, 136.50, 134.94, 110.76, 107.57, 106.53, 56.02, 53.61, 42.84, 40.28, 27.69. | found 286.1532 calculated 286.5131 C₁₄H₂₁N₃O₂Na |
| 60 | 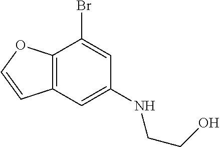 [SMILES: OCCNc1cc(Br)c2occc2c1] | ¹H NMR (400 MHz, CDCl3) δ 7.56 (d, J = 2.1, 1H), 6.83 (d, J = 2.1, 1H), 6.72 (d, J = 2.2, 1H), 6.66 (d, J = 2.2, 1H), 3.91-3.79 (m, 2H), 3.35-3.23 (m, 2H), 2.73 (bs, 1H). ¹³C NMR (101 MHz, CDCl3) δ 146.60, 146.22, 145.64, 129.46, 115.53, 107.35, 104.45, 103.01, 61.38, 47.24. | found 255.9975 calculated 255.9973 C₁₀H₁₁BrNO₂ |
| 61 | 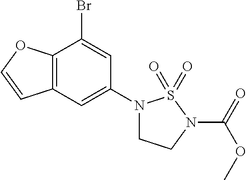 [SMILES: COC(=O)N1CCN(c2cc(Br)c3occc3c2)S1(=O)=O] | ¹H NMR (400 MHz, CDCl3) δ 7.72 (d, J = 2.1, 1H), 7.60 (d, J = 2.0, 1H), 7.50 (d, J = 2.0, 1H), 6.85 (d, J = 2.2, 1H), 4.02 (t, J = 6.3, 2H), 3.93 (s, 3H), 3.85 (t, J = 6.3, 2H). ¹³C NMR (101 MHz, CDCl3) δ 151.57, 151.30, 147.39, 132.26, 129.28, 123.72, 116.75, 107.97, 105.11, 54.77, 45.29, 42.71. | found 374.9664 calculated 374.9650 C₁₂H₁₂BrN₂O₅S |
| 62 | 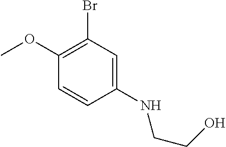 | ¹H NMR (400 MHz, CDCl3) δ 6.87 (d, J = 2.8, 1H), 6.77 (d, J = 8.8, 1H), 6.56 (dd, J = 2.8, 8.8, 1H), 3.80 (m, 6H, H1), 3.31- 3.14 (t, 2H). ¹³C NMR (101 MHz, CDCl3) δ 148.92, 143.51, 118.58, 114.20, 113.57, 112.93, 61.41, 57.32, 47.00. | found 246.0134 calculated 246.0130 C₉H₁₃BrNO₂ |
| 63 | 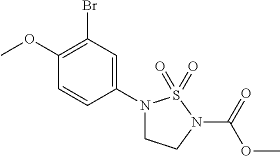 | ¹H NMR (500 MHz, CDCl3) δ 7.53 (d, J = 2.5, 1H), 7.33 (dd, J = 2.5, 8.8, 1H), 6.90 (d, J = 8.9, 1H), 3.97 (t, J = 6.3, 2H), 3.90 (s, 3H), 3.87 (s, 3H), 3.76 (dd, J = 5.7, 12.0, 2H). ¹³C NMR (126 MHz, DMSO) δ 195.85, 155.75, 151.28, 129.41, 129.18, 124.76, 112.59, 56.73, 54.64, 4 4.93, 42.68. | found 364.9820 calculated 364.9807 C₁₁H₁₄BrN₂O₅S |
| 64 | 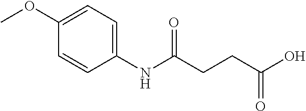 [SMILES: COc1ccc(NC(=O)CCC(=O)O)cc1] | ¹H NMR (400 MHz, MeOD) δ 7.56-7.29 (m, 2H), 6.96-6.75 (m, 2H), 4.90 (s, 1H), 3.74 (d, J = 2.2, 3H), 2.64 (d, J = 5.7, 4H). ¹³C NMR (101 MHz, CDC13) δ 176.42, 172.68, 157.92, 132.94, 123.13, 115.02, 55.96, 32.30, 30.22. | found 224.0922 calculated 224.0923 C₁₁H₁₄NO₄ |
| 65 | 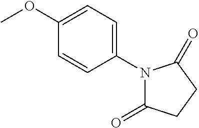 | ¹H NMR (500 MHz, CDCl3) δ 7.21-7.12 (m, 2H), 7.00-6.91 (m, 2H), 3.79 (s, 3H), 2.84 (s, 4H). ¹³C NMR (126 MHz, CDCl3) δ 176.90, 159.95, 128.11, 124.93, 114.96, 55.92, 28.79. | found 206.0809 calculated 206.0817 C₁₁H₁₂NO₃ |
| 66 | 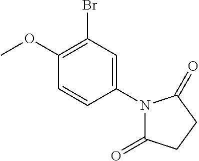 [SMILES: COc1ccc(N2C(=O)CCC2=O)cc1Br] | ¹H NMR (500 MHz, CDCl3) δ 7.49 (t, J = 10.1, 1H), 7.19 (dd, J = 2.5, 8.8, 1H), 6.95 (d, J = 8.8, 1H), 3.89 (s, 3H), 2.84 (s, 4H). ¹³C NMR (126 MHz, CDCl3) δ 176.90, 156.86, 132.23, 127.58, 126.00, 112.67, 78.17, 57.35, 29.21. | found 283.9931 calculated 283.9922 C₁₁H₁₁NO₃Br |
| 67 | 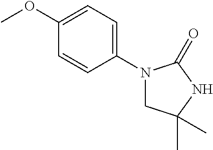 | ¹H NMR (400 MHz, CDCl3) δ 7.56-7.31 (m, 2H), 6.95-6.74 (m, 2H), 4.71 (s, 1H), 3.75 (d, J = 8.8, 3H), 3.57 (d, J = 8.8, 2H), 1.35 (d, J = 8.8, 6H). ¹³C NMR (101 MHz, CDCl3) δ 158.53, 155.65, 133.74, 120.04, 114.35, 110.30, 58.98, 55.74, 51.62, 28.86. | found 221.1294 calculated 221.1290 C₁₂H₁₇N₂O₂ |
| 68 | 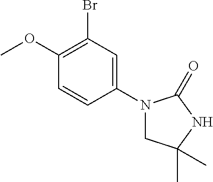 | ¹H NMR (400 MHz, CDCl3) δ 7.63 (d, J = 2.7, 1H), 7.50 (dd, J = 2.7, 8.9, 1H), 6.86 (d, J = 9.0, 1H), 4.79 (s, 1H), 3.85 (d, J = 7.8, 3H), 3.57 (s, 2H), 1.37 (s, 6H). ¹³C NMR (101 MHz, CDCl3) δ 158.12, 151.91, 134.65, 123.28, 118.66, 112.45, 111.85, 58.73, 56.80, 51.63, 28.89. | found 299.0394 calculated 299.0395 C₁₂H₁₆BrN₂O₂ |
| 69 | 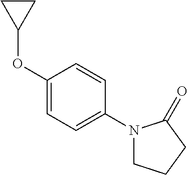 [SMILES: O=C1CCCN1c1ccc(OC2CC2)cc1] | ¹H NMR (500 MHz, CDCl3) δ 7.52-7.38 (d, J = 9.0, 2H), 7.09- 6.92 (d, J = 9.0, 2H), 3.87-3.75 (app. t, 2H), 3.73-3.57 (m, 1H), 2.67-2.46 (app. t, 1H), 2.21-2.03 (m, 1H), 0.75 (m, 2H). ¹³C NMR (126 MHz, CDCl3) δ 174.12, 156.09, 133.03, 121.98, 115.29, 51.13, 49.42, 32.66, 18.25, 6.41. | found 218.1180 calculated 218.1181 C₁₃H₁₆NO₂ |
| 70 | 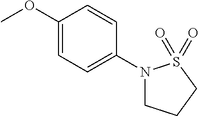 | ¹H NMR (400 MHz, CDCl3) δ 7.24 (d, J = 9.0, 2H), 6.89 (d, J = 9.0, 2H), 3.78 (s, 3H), 3.69 (t, J = 6.6, 2H), 3.33 (t, J = 7.6, 2H), 2.56-2.39 (m, 2H). ¹³C NMR (101 MHz, CDCl3) δ 157.91, 130.08, 124.05, 114.85, 55.67, 47.88, 47.73, 18.88. | found 250.0510 calculated 250.0514 C₁₀H₁₃NO₃SNa |
| 71 | 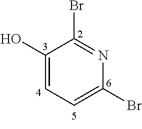 | 1H NMR: δH (500 MHz, MeOD) 7.36 (1H, d, J 8.5 Hz, H4), 7.14 (1H, d, J 8.5 Hz, H5); 13C NMR: δC (125 MHz, MeOD) 153.0 (C3), 130.3 (C), 129.0 (C4), 128. 7(C), 127.1 (C5); | found 251.8659 calculated 251.8660 C₅H₄⁷⁹Br₂NO |
| 72 | 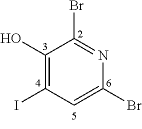 | 1H NMR: δH (500 MHz, CDCl₃) 7.78 (1H, s, H5), 5.93 (1H, s, OH); 13C NMR: δC (125 MHz, CDCl₃) 149.6 (C3), 137.0 (C5), 130.1 (C), 126.5 (C), 94.9 (C4); | found 377.7624 calculated 377.7626 C₅H₃⁷⁹Br₂INO |
| 73 | 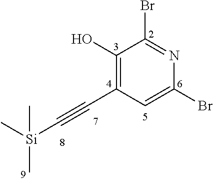 | 1H NMR: δH (500 MHz, CDCl₃) 7.36 (1H, s, H5), 5.93 (1H, s, OH), 0.28 (9H, s, H9); 13C NMR: δC (125 MHz, CDCl₃) 149.8 (C3), 129.2 (C), 129.1 (C5), 128.6 (C), 120.8 (C), 108.9 (C7 or C8), 94.8 (C7 or C8), −0.5 (C9); | found 347.9041 calculated 347.9049 C₁₀H₁₂⁷⁹Br₂NOSi |
| 74 | 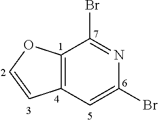 [SMILES: Brc1cc2ccoc2c(Br)n1] | 1H NMR: δH (500 MHz, CDCl₃) 7.81 (1H, d, J 2.0 Hz, H2), 7.24 (1H, s, H5), 6.83 (1H, d, J 2.0 Hz, H3); 13C NMR: δC (125 MHz, CDCl₃) 150.0 (C2), 149.7 (C), 137.7 (C), 131.4 (C), 122.7 (C), 119.5 (C5), 106.7 (C3); | found 275.8656 calculated 275.8660 C₇H₄NO⁷⁹Br₂ |
| 75 | 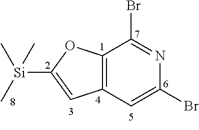 | 1H NMR: δH (500 MHz, CDCl₃) 7.60 (1H, s, H5), 6.95 (1H, s, H3), 0.39 (9H, s, H8); 13C NMR: δC (125 MHz, CDCl₃) 171.6 (C), 152.6 (C), 138.3 (C), 130.6 (C), 122.5 (C), 118.9 (C5), 115.1 (C3), −2.1 (C8); | found 347.9034, calculated 347.9049 C₁₀H₁₂⁷⁹Br₂NOSi |
| 76 | 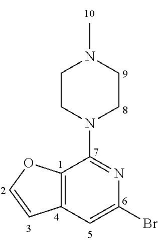 [SMILES: CN1CCN(c2nc(Br)cc3ccoc23)CC1] | 1H NMR: δH (500 MHz, CDCl₃), 7.57 (1H, d, J 2.0 Hz, H2), 7.00 (1H, s, H5), 6.61 (1H, d, J 2.0 Hz, H3), 3.88 (4H, t, J 5.0 Hz, H8), 2.53 (4H, t, J 5.0 Hz, H9), 2.33 (3H, s, H10); 13C NMR: δC (125 MHz, CDCl₃) 146.5 (C2), 145.1 (C), 139.8 (C), 137.6 (C), 130.7 (C), 109.3 (C5), 106.1 (C3), 55.0 (C8), 46.2 (C10), 45.9 (C9); | found 296.0400 calculated 296.0398 C₁₂H₁₅N₃O⁷⁹Br |
| 77 | 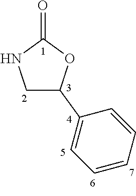 [SMILES: O=C1NCC(c2ccccc2)O1] | 1H NMR: δH (500 MHz, CDCl₃) 7.41-7.32 (5H, m, H5, H6, H7), 5.98 (1H, br s, NH), 5.60 (1H, app t, J 8.0 Hz, H3), 3.96 (1H, app t, J 8.5 Hz, H2a), 3.53 (1H, app t, J 8.5 Hz, H2b); 13C NMR: δC (125 MHz, CDCl₃) 159.8 (C1), 138.4 (C4), 128.92 (CH), 128.90 (CH), 125.7 (CH), 77.9 (C3), 48.3 (C2). | |
| 78 | 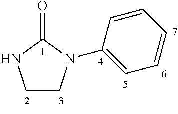 [SMILES: O=C1NCCN1c1ccccc1] | 1H NMR: δH (500 MHz, MeOD) 7.51-7.47 (2H, m, H5), 7.32-7.26 (2H, m, H6), 7.02 (1H, tt, J 7.5, 1.0 Hz, H7), 3.92 (2H, dd, J 9.0, 7.0 Hz, H2 or H3), 3.54-3.43 (2H, m, H2 or H3); 13C NMR: δC (125 MHz, MeOD) 162.2 (C1), 141.6 (C4), 129.7 (CH), 123.9 (CH), 119.5 (CH), 46.7 (C2 or C3), 38.5 (C2 or C3); | found 163.0874 calculated 63.0871 C₉H₁₁N₂O |

Compounds of formula (I) may also be prepared from other compounds of formula (I) by well-known methods.

### BRIEF DESCRIPTION OF FIGURES

FIG. 1 includes scheme 1, describing the synthesis of 5a.

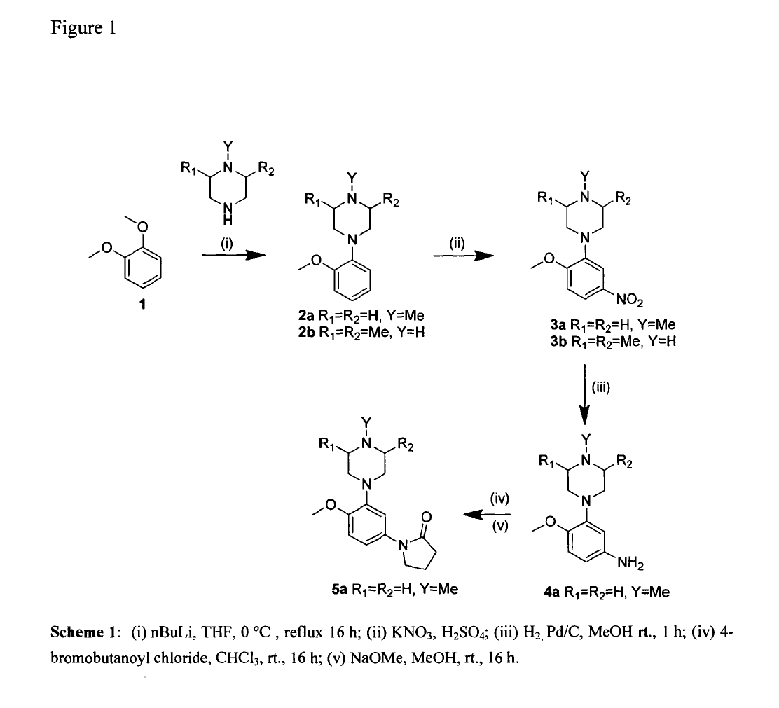

FIG. 2 includes schemes 2 and 3, describing the synthesis of 7 and 13a-e, respectively.

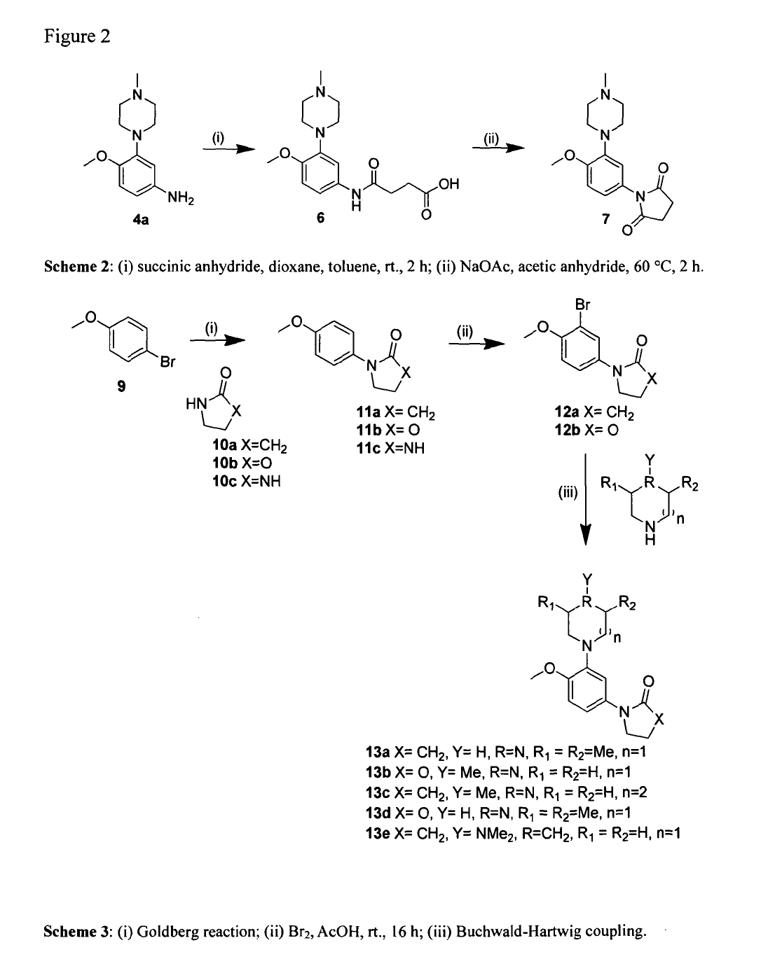

FIG. 3 includes scheme 4 and 5, describing the synthesis of 13f and 18a-b, respectively.

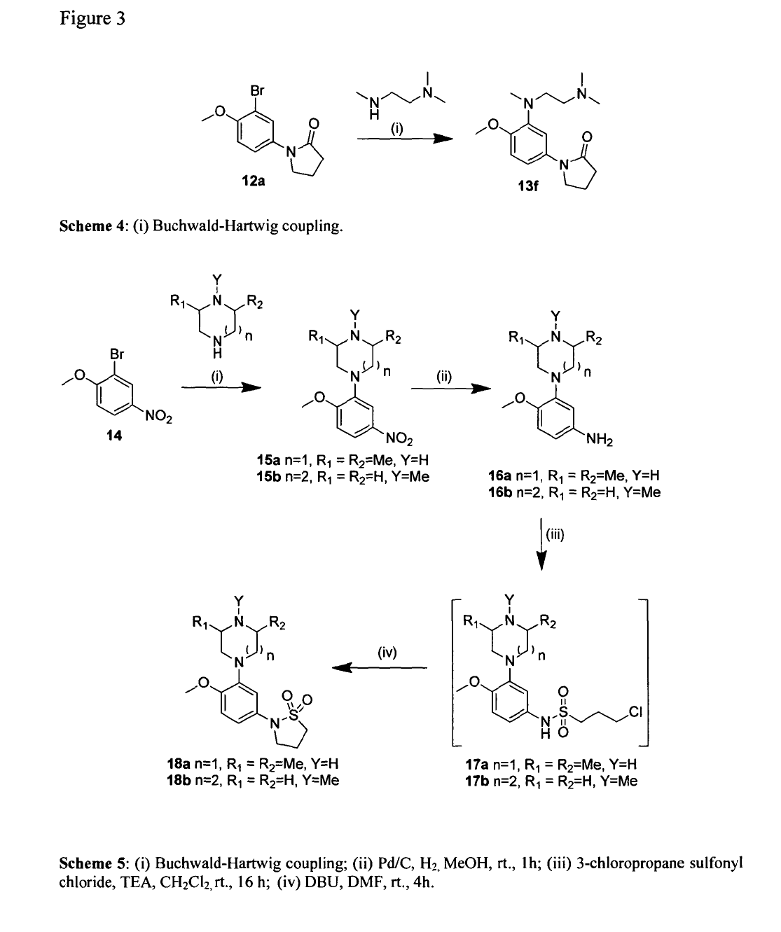

FIG. 4 includes scheme 6 describing the synthesis of 22.

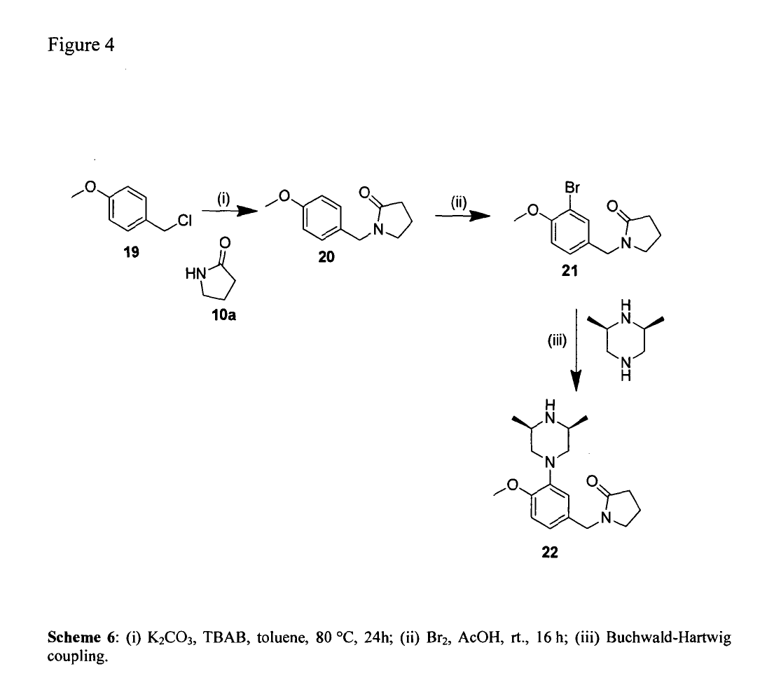

FIG. 5 includes scheme 7 describing the synthesis of 26a-c.

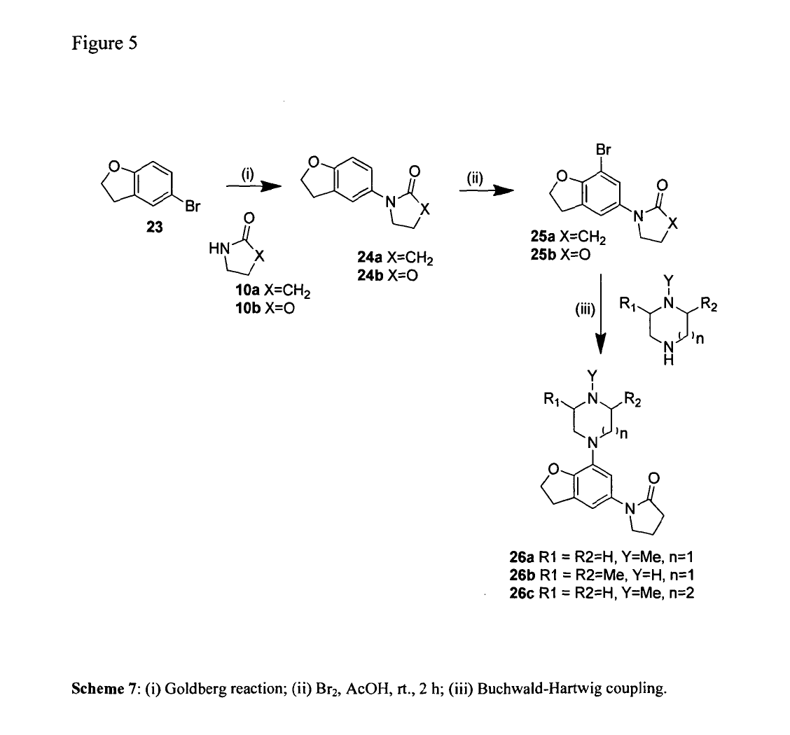

FIG. 6 includes scheme 8 describing the synthesis of 30 and 33a-c.

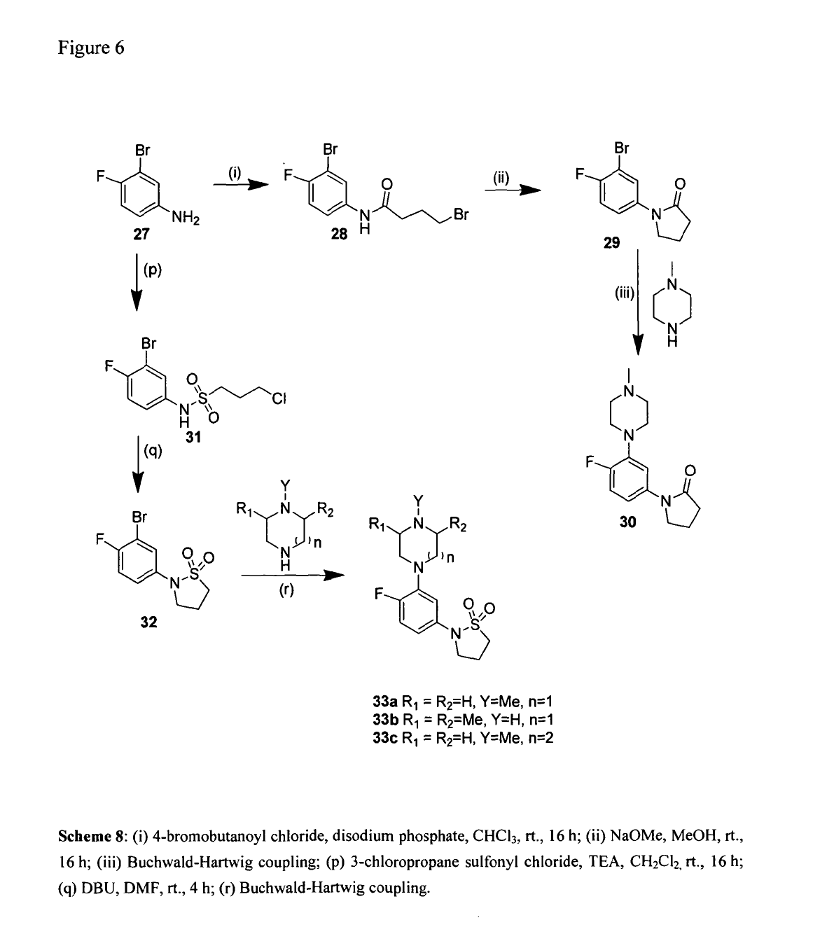

FIG. 7 includes scheme 9 describing the synthesis of 40.

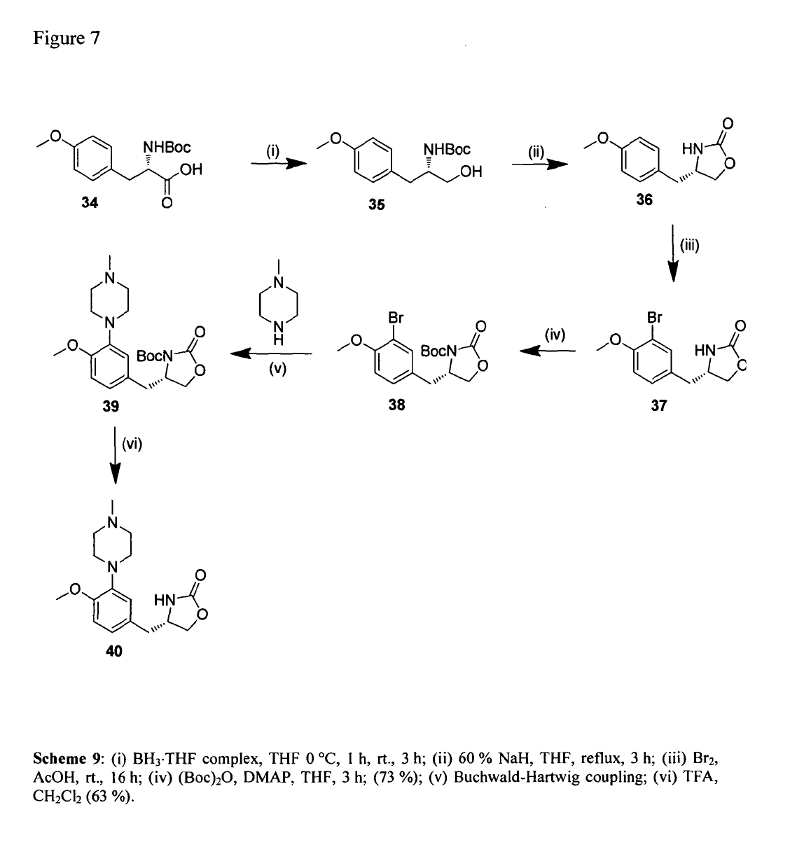

FIG. 8 includes scheme 10 and 11, describing the synthesis of 45 and 47a-b, 49a and 50a respectively.

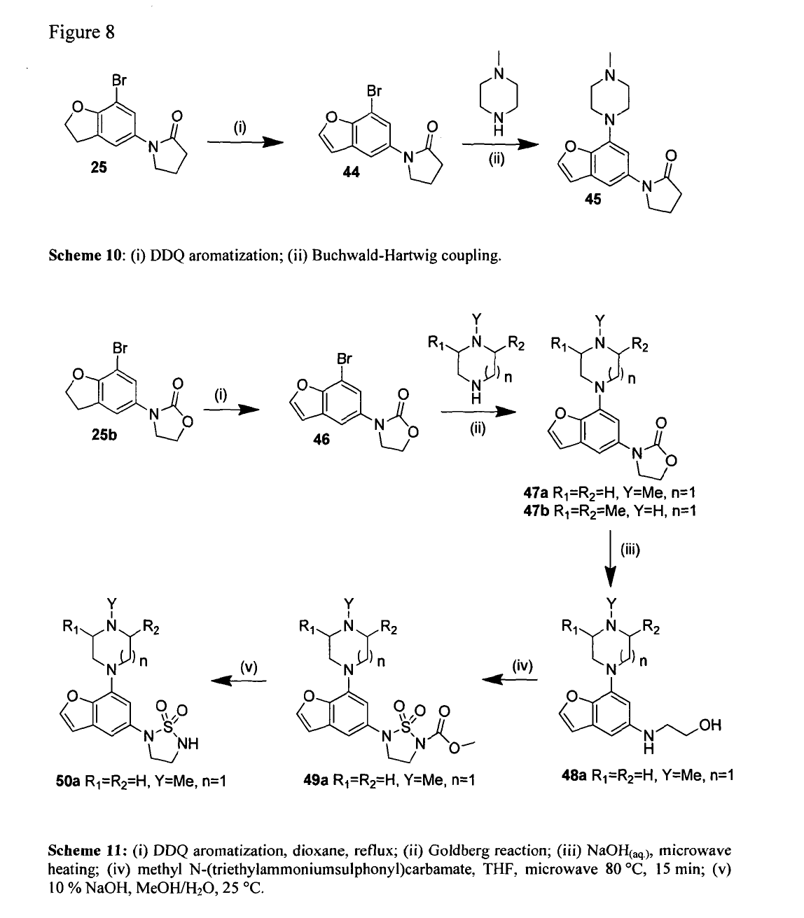

FIG. 9 includes schemes 12 and 13, describing the synthesis of 55 and 59, respectively.

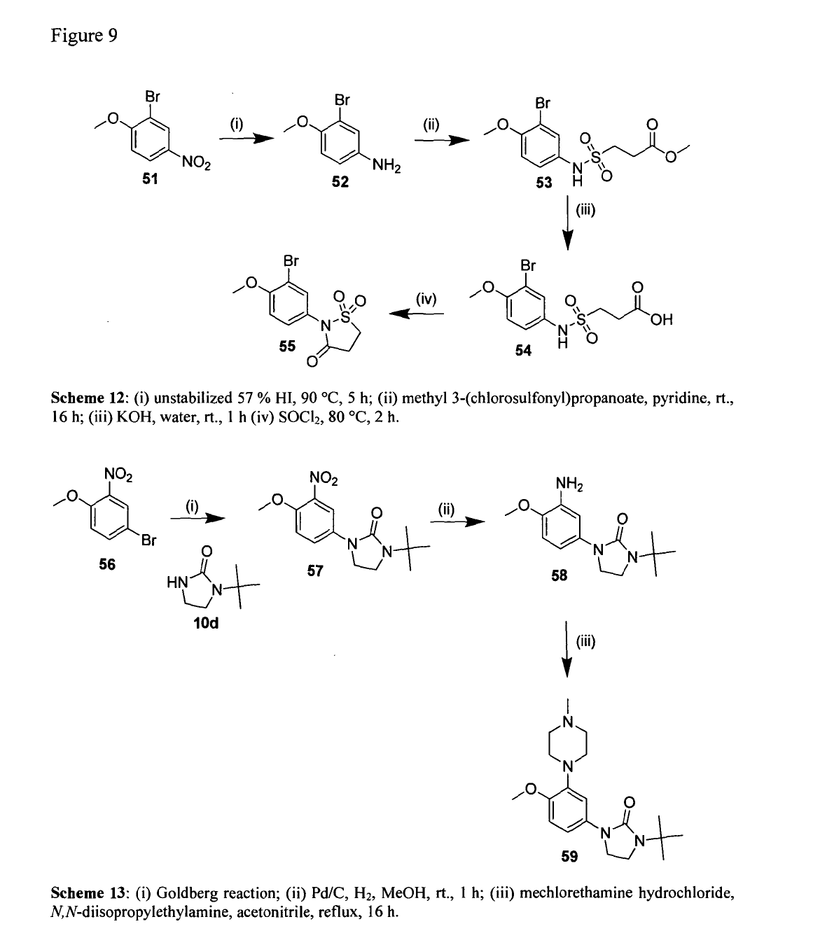

FIG. 10 includes scheme 14, describing the synthesis of 79, 80, 81, 82, 83 & 84 respectively.

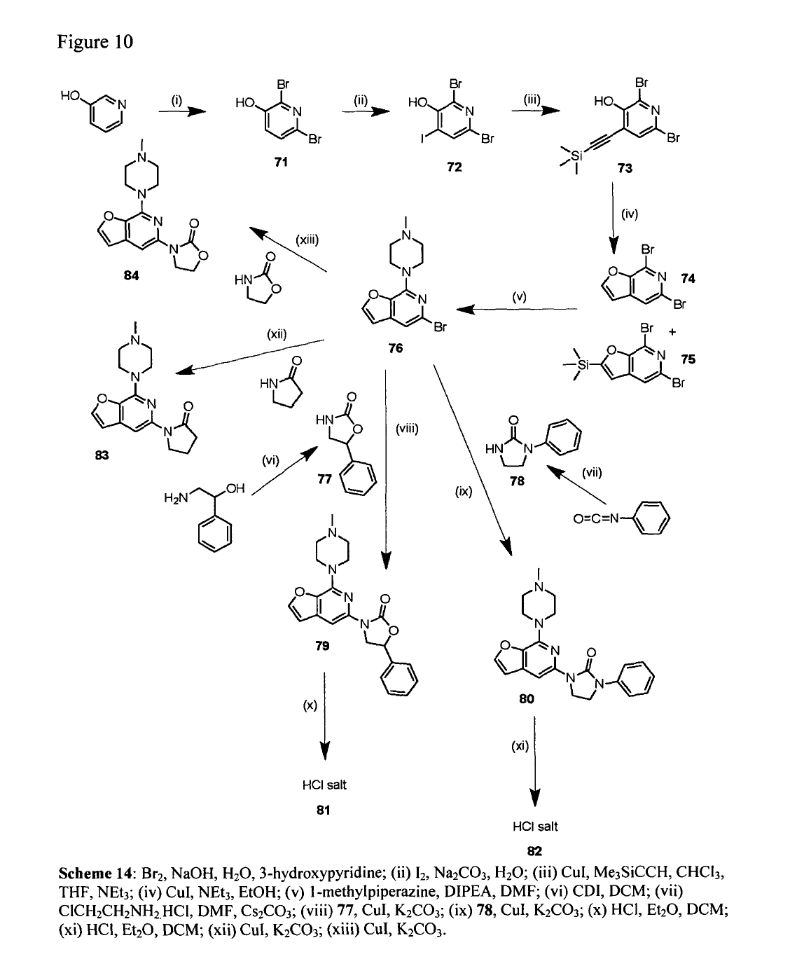

FIG. 11 illustrates representative guinea-pig functional assay data for 13a.

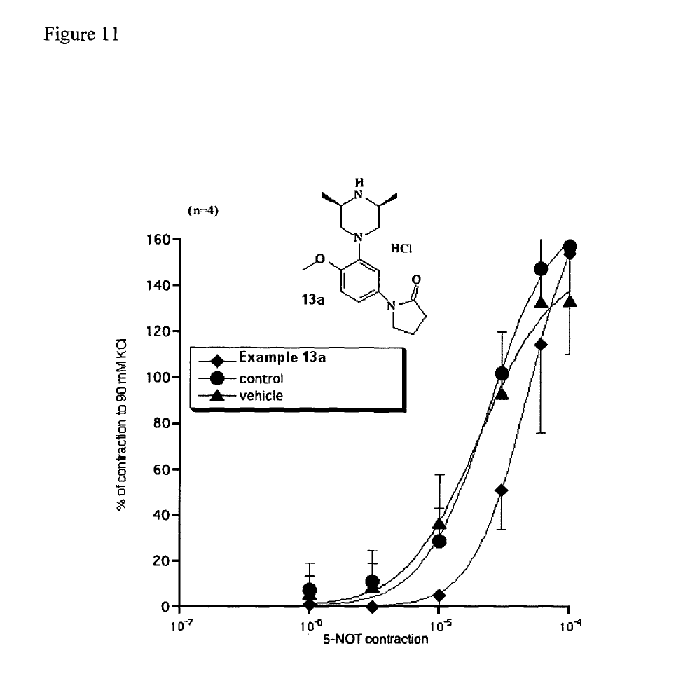

FIG. 12 illustrates the crystal structures of 47a and 49a obtained by single crystal X-ray diffraction.

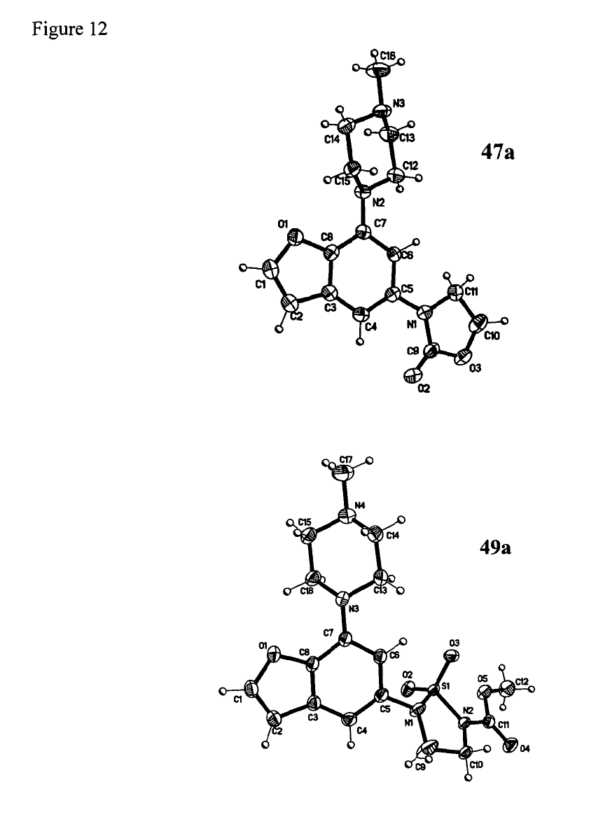

FIG. 13 illustrates representative functional assay data showing the effect of 82 (GMH029) (15 mg/kg/day) on chronic hypoxia-induced increases in systolic right ventricular pressure (sRVP).

FIG. 14 illustrates representative functional assay data showing the effect of 82 (GMH029) (15 mg/kg/day) on chronic hypoxia-induced right ventricular hypertrophy (RVH).

FIG. 15 illustrates representative functional assay data showing the effect of 82 (GMH029) (15 mg/kg/day) on mean systemic arterial pressure (mSAP).

FIG. 16 illustrates representative functional assay data showing the effect of 82 (GMH029) (15 mg/kg/day) on heart rate (HR).

FIG. 17 illustrates representative functional assay data showing the effect of 82 (GMH029) (15 mg/kg/day) on chronic hypoxia-induced increases in vasoreactivity to 5-HT.

### MODES FOR CARRYING OUT THE INVENTION

The following Examples are intended to illustrate the invention and are not to be construed as being limitations thereon. If not mentioned otherwise, all evaporations are performed under reduced pressure, between about 50 mmHg and 100 mmHg. The structure of final products, intermediates and starting materials is confirmed by standard analytical methods, e.g., microanalysis, melting point (m.p.) and spectroscopic characteristics, e.g. MS, IR and NMR. Abbreviations used are those conventional in the art.

Table 2 provides comparative compounds that have been prepared by the synthetic methods described above.

TABLE 2

| Comparative | | | |
|---|---|---|---|
| Example | | | (M + H)⁺ or |
| Structure No. | Structure and name | ¹H & ¹³C NMR | (M + Na)⁺ |
| 13e | 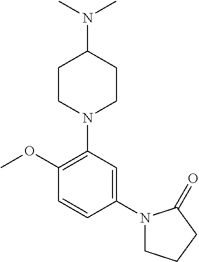 [SMILES: COc1ccc(N2CCCC2=O)cc1N1CCC(N(C)C)CC1] 1-(3-(4-(dimethylamino)piperidin- 1-yl)-4-methoxyphenyl)pyrrolidin- 2-one | ¹H NMR (400 MHz, CDCl₃) δ ppm 7.32 (d, J = 2.56 Hz, 1H), 6.97 (dd, J = 8.71, 2.57 Hz, 1H), 6.77 (d, J = 8.75 Hz, 1H), 3.90- 3.69 (m, 5H), 3.52 (d, J = 11.83 Hz, 2H), 2.62-2.46 (m, 4H), 2.30 (s, 7H), 2.15-2.02 (m, 2H), 1.86 (d, J = 12.03 Hz, 2H), 1.72 (ddd, J = 12.10, 3.66 Hz, 2H) ¹³C NMR (101 MHz, CDCl₃) δ 173.88, 149.38, 141.84, 132.96, 114.31, 111.86, 111.10, 62.20, 55.61, 50.55, 49.27, 41.38, 32.57, 28.22, 17.98 | Found 318.218 Calculated 318.2182 C₁₈H₂₈N₃O₂ |
| 13f | 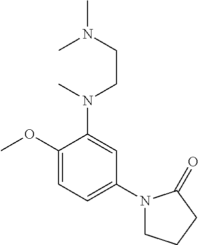 [SMILES: COc1ccc(N2CCCC2=O)cc1N(C)CCN(C)C] 1-(3-((2-(dimethylamino)ethyl) (methyl)amino)-4- methoxyphenyl)pyrrolidin- 2-one | ¹H NMR (400 MHz, CDCl3) δ 7.28 (d, J = 2.6, 1H), 6.98 (dd, J = 2.6, 8.7, 1H), 6.77 (d, J = 8.7, 1H), 3.84-3.76 (m, 5H), 3.21- 3.10 (m, 2H), 2.78 (s, 3H), 2.55 (t, J = 8.1, 2H), 2.47 (dd, J = 6.6, 8.4, 2H), 2.21 (s, 6H), 2.10 (dt, J = 7.5, 15.3, 2H). ¹³C NMR (101 MHz, CDCl3) δ 174.14, 149.72, 141.93, 133.00, 114.15, 112.49, 111.28, 57.17, 55.73, 53.41, 49.53, 45.98, 40.68, 32.76, 18.21. | Found 292.2027 Calculated 292.2025 C₁₆H₂₆N₃O₂ |

Table 3 provides a list of compounds of formula (I) that have been prepared by the synthetic methods described above.

TABLE 3

| Example | | | (M + H)⁺ or |
|---|---|---|---|
| No. | Structure | ¹H & ¹³C NMR | (M + Na)⁺ |
| 5a | 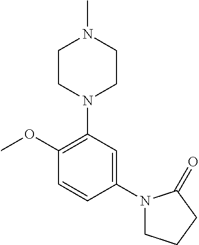 [SMILES: COc1ccc(N2CCCC2=O)cc1N1CCN(C)CC1] 1-(4-methoxy-3-(4-methylpiperazin-1- yl)phenyl)pyrrolidin-2-one | ¹H NMR (400 MHz, CDCl3) δ 7.40 (d, J = 3.3, 1H), 7.22 (dd, J = 8.7, 26.5, 1H), 6.92 (dd, J = 8.5, 19.2, 1H), 4.08-3.89 (m, 4H), 3.76 (t, J = 6.0, 1H), 3.24 (s, 4H), 2.91-2.55 (m, 7H), 2.48 (s, 3H), 2.36-2.17 (m, 2H). ¹³C NMR (101 MHz, CDCl3) δ 173.93, 149.27, 133.03, 115.01, 111.80, 111.27, 110.06, 55.62, 54.92, 49.67, 49.27, 45.50, 32.51, 18.00 | Found 290.1881 Calculated 290.1869 C₁₆H₂₄N₃O₂ |
| 7 | 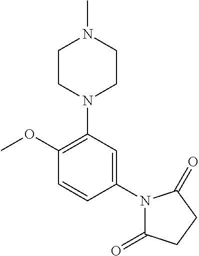 [SMILES: COc1ccc(N2C(=O)CCC2=O)cc1N1CCN(C)CC1] 1-(4-methoxy-3-(4-methylpiperazin-1- yl)phenyl)pyrrolidine-2,5-dione | ¹H NMR (500 MHz, CDCl3) δ 6.87 (d, J = 8.6, 1H), 6.83 (dd, J = 2.3, 8.6, 1H), 6.74 (d, J = 2.3, 1H), 3.83 (s, 3H), 3.07 (s, 4H), 2.81 (s, 5H), 2.59 (s, 4H), 2.29 (d, J = 21.0, 3H). ¹³C NMR (126 MHz, CDCl3) δ 176.70, 152.41, 141.96, 124.94, 120.94, 117.00, 111.49, 55.82, 55.20, 50.33, 46.08, 28.50. | Found 304.1659 Calculated 304.1661 C₁₆H₂₂N₃O₃ |
| 13a | 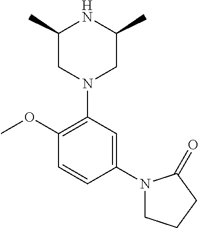 [SMILES: COc1ccc(N2CCCC2=O)cc1N1C[C@@H](C)N[C@@H](C)C1] 1-(3-((3R,5S)-3,5-dimethylpiperazin-1- yl)-4-methoxyphenyl)pyrrolidin-2-one | ¹H NMR (500 MHz, CDCl3) δ 7.19 (d, J = 2.5, 1H), 7.08 (dd, J = 2.5, 8.8, 1H), 6.79 (d, J = 8.8, 1H), 3.85-3.70 (m, 5H), 3.40 (dd, J = 8.7, 15.0, 4H), 2.79 (t, J = 11.6, 2H), 2.54 (t, J = 8.1, 2H), 2.19-2.02 (m, 2H), 1.42 (d, J = 6.4, 6H). ¹³C NMR (126 MHz, CDCl3) δ 173.95, 149.23, 139.73, 132.89, 115.61, 112.26, 111.46, 55.71, 54.52, 52.25, 49.26, 32.40, 17.91, 16.84. | found 304.2027 calculated 304.2025 C₁₇H₂₆N₃O₂ |
| 13b | 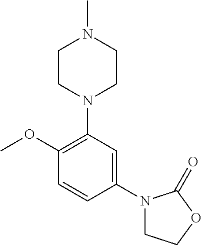 [SMILES: COc1ccc(N2CCOC2=O)cc1N1CCN(C)CC1] 3-(4-methoxy-3-(4-methylpiperazin-1- yl)phenyl)oxazolidin-2-one | ¹H NMR (500 MHz, MeOD) δ 7.46 (d, J = 2.6, 1H), 7.12 (dd, J = 2.6, 8.8, 1H), 7.03 (d, J = 8.9, 1H), 4.62-4.36 (m, 2H), 4.18- 4.00 (m, 2H), 3.88 (s, 3H), 3.76- 3.46 (m, 4H), 3.36 (d, J = 12.1, 2H), 3.25-3.07 (m, 2H), 2.97 (s, 3H). ¹³C NMR (126 MHz, MeOD) δ 158.29, 150.90, 139.81, 133.57, 116.75, 113.55, 112.72, 63.44, 56.59, 54.96, 49.22, 47.32, 43.83, 31.93. | Found 292.1628 Calculated 292.1661 C₁₅H₂₂N₃O₃ |
| 13c | 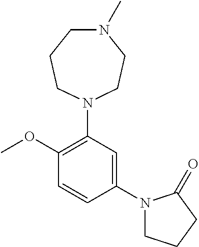 [SMILES: COc1ccc(N2CCCC2=O)cc1N1CCCN(C)CC1] 1-(4-methoxy-3-(4-methyl-1,4- diazepan-1-yl)phenyl)pyrrolidin-2-one | ¹H NMR (400 MHz, CDCl3) δ 7.27 (d, J = 2.5, 1H), 6.89 (dd, J = 2.5, 8.7, 1H), 6.76 (d, J = 8.8, 1H), 3.83-3.68 (m, 5H), 3.42 (d, J = 4.4, 5H), 3.25 (s, 2H), 2.82 (s, 3H), 2.50 (t, J = 8.1, 4H), 2.15-2.01 (m, 2H). ¹³C NMR (101 MHz, CDCl3) δ 174.20, 148.75, 140.98, 133.04, 114.04, 111.84, 111.29, 59.28, 55.71, 54.77, 49.42, 49.12, 48.36, 44.57, 32.60, 24.04, 18.05. | Found 304.2032 Calculated 304.2025 C₇H₂₇N₃O₂ |
| 13d | 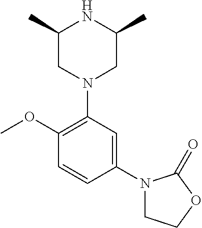 [SMILES: COc1ccc(N2CCOC2=O)cc1N1C[C@@H](C)N[C@@H](C)C1] 3-(3-((3R,5S)-3,5-dimethylpiperazin-1- yl)-4-methoxyphenyl)oxazolidin-2-one | ¹H NMR (500 MHz, CDCl3) δ 7.21 (d, J = 2.7, 1H), 6.94 (dd, J = 2.7, 8.7, 1H), 6.80 (d, J = 8.8, 1H), 4.42 (dd, J = 7.2, 8.8, 2H), 4.00 (dd, J = 7.2, 8.8, 2H), 3.82 (s, 3H), 3.36 (d, J = 9.7, 2H), 3.19-3.04 (m, 2H), 2.22 (t, J = 10.8, 2H), 2.01 (bs, 1H), l.10 (d, J = 6.4, 6H). ¹³C NMR (126 MHz, CDCl3) δ 155.77, 149.38, 141.94, 132.04, 113.06, 111.61, 110.64, 61.43, 57.75, 55.91, 51.03, 46.04, 19.88. | Found 306.1815 Calculated 306.1818 C₁₆H₂₄N₃O₃ |
| 18a | 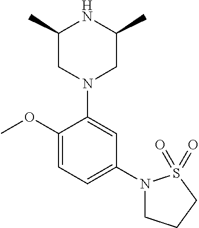 [SMILES: COc1ccc(N2CCCS2(=O)=O)cc1N1C[C@@H](C)N[C@@H](C)C1] 2-(3-((3S,5R)-3,5-dimethylpiperazin-1- yl)-4-methoxyphenyl)-1,1- dioxoisothiazolidine | ¹H NMR (500 MHz, CDCl3) δ 6.93 (dd, J = 2.6, 8.7, 1H), 6.85 (d, J = 2.6, 1H), 6.81 (d, J = 8.7, 1H), 5.72 (bs, 1H), 3.81 (s, 3H), 3.69 (t, J = 6.6, 2H), 3.49-3.25 (m, 7H), 2.62 (t, J = 11.3, 2H), 2.53-2.39 (m, 2H), 1.28 (d, J = 6.4, 6H). ¹³C NMR (126 MHz, CDCl3) δ 151.22, 141.54, 130.93, 118.03, 114.65, 112.67, 56.47, 55.73, 52.15, 48.42, 48.24, 19.33, 17.97. | found 340.1695 calculated 340.1695 C₁₆H₂₆N₃O₃S |
| 18b | 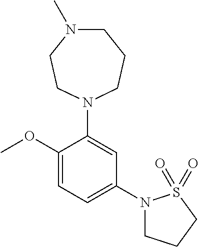 [SMILES: COc1ccc(N2CCCS2(=O)=O)cc1N1CCCN(C)CC1] 2-(4-methoxy-3-(4-methyl-1,4- diazepan-1-yl)phenyl)-1,1- dioxoisothiazolidine | ¹H NMR (400 MHz, CDCl3) δ 6.85 (d, J = 2.4, 1H), 6.77 (d, J = 8.6, 1H), 6.73 (dd, J = 2.4, 8.6, 1H), 3.78 (s, 3H), 3.68 (t, J = 6.6, 2H), 3.40-3.24 (m, J = 4.2, 8.0, 9.6, 6H), 2.77 (dd, J = 3.7, 5.9, 2H), 2.73-2.63 (m, 2H), 2.51-2.40 (m, 2H), 2.39 (s, 3H), 2.06-1.91 (m, 2H). ¹³C NMR (101 MHz, CDCl3) δ 150.04, 143.50, 130.37, 114.45, 112.96, 112.23, 59.29, 57.01, 55.91, 52.38, 51.56, 47.95, 47.85, 46.91, 28.17, 18.91. | found 340.1707 calculated 340.1695 C₁₆H₂₆N₃O₃S |
| 22 | 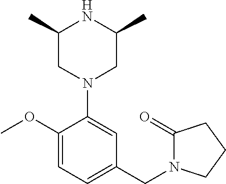 [SMILES: COc1ccc(CN2CCCC2=O)cc1N1C[C@@H](C)N[C@@H](C)C1] 1-(3-((3S,5R)-3,5-dimethylpiperazin-1- yl)-4-methoxybenzyl)pyrrolidin-2-one | ¹H NMR (500 MHz, CDCl3) δ 6.79 (dd, J = 2.0, 8.2, 1H), 6.76- 6.69 (m, 2H), 4.31 (s, 2H). 3.79 (s, 3H), 3.28 (d, J = 9.6, 2H), 3.21-3.14 (m, 2H), 3.14- 3.01 (m, 2H), 2.37 (t, J = 8.1, 2H), 2.12 (t, J = 10.7, 2H), 1.91 (dt, J = 7.5, 15.4, 2H), 1.75 (bs, 1H), 1.05 (d, J = 6.4, 6H). ¹³C NMR (126 MHz, CDCl3) δ 174.84, 151.75, 141.63, 129.03, 122.61, 118.48, 111.26, 57.95, 55.59, 50.83, 50.33, 46.59, 46.41, 31.16, 19.92, 17.79. | found 318.2195 calculated 318.2182 C₁₈H₂₆N₃O₂ |
| 26a | 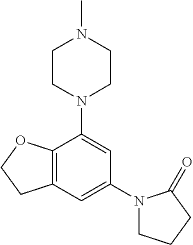 [SMILES: CN1CCN(c2cc(N3CCCC3=O)cc3c2OCC3)CC1] 1-(7-(4-methylpiperazin-1-yl)-2,3- dihydrobenzofuran-5-yl)pyrrolidin-2-one | ¹H NMR (500 MHz, CDCl3) δ 6.99 (s, 1H), 6.88 (d, J = 2.0, 1H), 4.57 (t, J = 8.8, 2H), 3.78 (t, J = 7.0, 2H), 3.24-3.08 (m, J = 8.8, 6H), 2.64-2.48 (m, 6H), 2.32 (s, 3H), 2.11 (dt, J = 7.5, 15.3, 2H). ¹³C NMR (126 MHz, CDCl3) δ 174.15, 148.52, 136.18, 133.26, 127.82, 111.56, 109.58, 71.50, 55.30, 50.11, 49.56, 46.39, 32.71, 30.50, 18.34. | found 302.1869 calculated 302.1869 C₁₇H₂₄N₃O₂ |
| 26b | 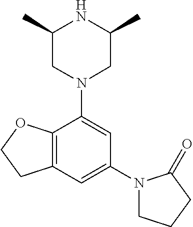 [SMILES: C[C@@H]1CN(c2cc(N3CCCC3=O)cc3c2OCC3)C[C@H](C)N1] 1-(7-((3S,5R)-3,5-dimethylpiperazin-1- yl)-2,3-dihydrobenzofuran-5- yl)pyrrolidin-2-one | ¹H NMR (500 MHz, CDCl3) δ 6.96 (d, J = 1.3, 1H), 6.86 (d, J = 1.9, 1H), 4.56 (t, J = 8.8, 2H), 3.78 (t, J = 7.0, 2H), 3.46 (d, J = 9.5, 2H), 3.16 (t, J = 8.7, 2H), 3.12-3.03 (m, 2H), 2.54 (t, J = 8.1, 2H), 2.19 (t, J = 10.8, 2H), 2.16-2.03 (m, 2H), 1.88 (s, 1H), 1.09 (d, J = 6.4, 6H). ¹³C NMR (126 MHz, CDCl3) δ 174.14, 148.54, 136.17, 133.20, 127.79, 111.49, 109.81, 71.47, 56.58, 50.86, 50.79, 50.12, 32.68, 30.46, 19.89, 18.30. | found 316.2025 calculated 316.2025 C₁₈H₂₆N₃O₂ |
| 26c | 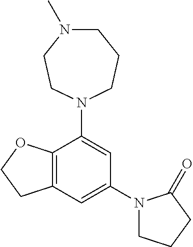 [SMILES: CN1CCCN(c2cc(N3CCCC3=O)cc3c2OCC3)CC1] 1-(7-(4-methyl-1,4-diazepan-1-yl)-2,3- dihydrobenzofuran-5-yl)pyrrolidin-2-one | ¹H NMR (500 MHz, CDCl3) δ 6.80 (d, J = 2.1, 1H), 6.78-6.68 (m, 1H), 4.48 (t, J = 8.7, 2H), 3.76 (t, J = 7.0, 2H), 3.56-3.48 (m, 2H), 3.40 (t, J = 6.3, 2H), 3.12 (t, J = 8.7, 2H), 2.71 (dd, J = 3.8, 5.7, 2H), 2.64-2.58 (m, 2H), 2.53 (t, J = 8.1, 2H), 2.36 (s, 3H), 2.13-2.04 (m, 2H), 2.00-1.92 (m, 2H). ¹³C NMR (126 MHz, CDCl3) δ 174.12, 146.44, 136.53, 133.28, 127.69, 108.50, 108.28, 71.01, 59.59, 57.09, 50.89, 50.27, 50.16, 46.97, 32.70, 30.70, 28.21, 18.32. | found 316.2025 calculated 316.2025 C₁₈H₂₆N₃O₂ |
| 30 | 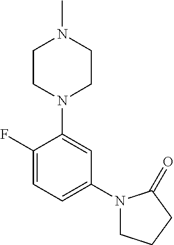 [SMILES: CN1CCN(c2cc(N3CCCC3=O)ccc2F)CC1] 1-(4-fluoro-3-(4-methylpiperazin-1- yl)phenyl)pyrollidin-2-one | ¹H NMR (500 MHz, CDCl3) δ 7.38 (dd, J = 2.5, 7.9, 1H), 7.00- 6.88 (m, 2H), 3.80 (t, J = 7, 0, 2H), 3.23-3.02 (m, 4H), 2.57 (t, J = 8.1, 6H), 2.32 (s, 3H), 2.23-2.04 (m, 2H). ¹³C NMR (126 MHz, CDCl3) δ 174.30, 153.60, 151.65, 140.33, 140.26, 136.10, 136.08, 116.18, 116.00, 113.68, 113.62, 111.90, 111.88, 55.33, 50.54, 50.51, 49.36, 46.35, 32.83, 18.17. | found 278.1675 calculated 278.1669 C₁₅H₂₁FN₃O |
| 33a | 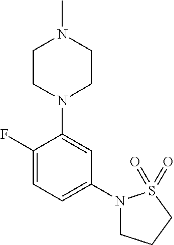 [SMILES: CN1CCN(c2cc(N3CCCS3(=O)=O)ccc2F)CC1] 2-(4-fluoro-3-(4-methylpiperazin-1- yl)phenyl)-1,1-dioxoisothiazolidine | ¹H NMR (500 MHz, CDCl3) δ 6.97 (dd, J = 8.7, 12.2, 1H), 6.87 (dd, J = 2.7, 7.6, 1H), 6.80- 6.71 (m, 1H), 3.70 (t, J = 6.6, 2H), 3.40-3.28 (m, 2H), 3.18- 3.06 (m, 4H), 2.56 (s, 4H), 2.52- 2.43 (m, 2H), 2.33 (s, 3H). ¹³C NMR (126 MHz, CDCl3) δ 154.43, 152.48, 140.94, 140.87, 133.86, 133.84, 116.86, 116.68, 114.58, 114.52, 112.70, 112.68, 55.28, 50.39, 50.36, 48.10, 47.62, 46.35, 18.92. | found 314.1336, calculated 314.1339 C₁₄H₂₁FN₃O₂S |
| 33b | 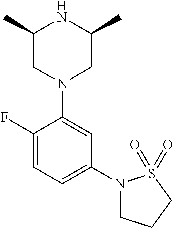 [SMILES: C[C@@H]1CN(c2cc(N3CCCS3(=O)=O)ccc2F)C[C@H](C)N1] 2-(3-((3S,5R)-3,5-dimethylpiperazin-1- yl)-4-fluorophenyl)-1,1- dioxoisothiazolidine | ¹H NMR (500 MHz, CDCl3) δ 6.96 (dd, J = 8.7, 12.2, 1H), 6.85 (dd, J = 2.7, 7.6, 1H), 6.79- 6.72 (m, 1H), 3.70 (t, J = 6.6, 2H), 3.40-3.24 (m, 4H), 3.15- 3.00 (m, 2H), 2.55-2.40 (m, 2H), 2.30 (t, J = 10.8, 2H), 1.76 (s, 1H), 1.09 (d, J = 6.4, 6H). ¹³C NMR (126 MHz, CDCl3) δ 154.44, 152.50, 140.96, 140.89, 133.80, 133.78, 116.82, 116.64, 114.72, 114.66, 113.01, 112.98, 57.42, 57.39, 50.94, 48.07, 47.67, 19.80, 18.90. | Found 328.1506 calculated 328.1495 C₁₅H₂₃FN₃O₂S |
| 33c | 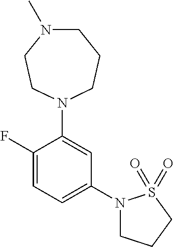 [SMILES: CN1CCCN(c2cc(N3CCCS3(=O)=O)ccc2F)CC1] 2-(4-fluoro-3-(4-methyl-1,4-diazepan-1- yl)phenyl)-1,1-dioxoisothiazolidine | ¹H NMR (500 MHz, CDCl3) δ 6.91 (dd, J = 8.6, 13.3, 1H), 6.78 (dd, J = 2.7, 8.0, 1H), 6.53 (dt, J = 3.1, 8.6, 1H), 3.67 (t, J = 6.6, 2H), 3.45-3.39 (m, 2H), 3.36 (t, J = 6.3, 2H), 3.34-3.29 (m, 2H), 2.80-2.69 (m, 2H), 2.66- 2.59 (m, 2H), 2.51-2.40 (m, 2H), 2.37 (s, 3H), 2.05-1.94 (m, 2H). ¹³C NMR (126 MHz, CDCl3) δ 152.48, 150.56, 140.86, 140.79, 133.67, 133.66, 116.93, 116.75, 111.21, 111.15, 110.84, 110.80, 59.34, 59.33, 57.00, 51.57, 51.53, 50.65, 50.63, 48.08, 47.60, 46.85, 28.26, 18.89. | found 328.1499 calculated 328.1495 C₁₅H₂₃FN₃O₂S |
| 40 | 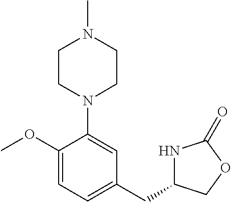 [SMILES: COc1ccc(C[C@H]2COC(=O)N2)cc1N1CCN(C)CC1] (S)-4-(4-methoxy-3-(4-methylpiperazin- 1-yl)benzyl)oxazolidin-2-one | ¹H NMR (500 MHz, CDCl3) δ 6.83-6.72 (m, 2H), 6.67 (s, 1H), 5.16 (s, 1H), 4.44 (t, J = 8.3, 1H), 4.11 (dd, J = 5.6, 8.5, 1H), 4.07-3.96 (m, 1H), 3.83 (s, 3H), 3.07 (s, 4H), 2.86-2.69 (m, 2H), 2.60 (s, 4H), 2.34 (s, 3H). ¹³C NMR (126 MHz, CDCl3) δ 159.18, 151.64, 141.99, 128.62, 123.12, 118.85, 111.78, 69.92, 55.74, 55.49, 54.15, 50.72, 46.34, 41.29. | found 306.1815 calculated 306.1818 C₁₆H₂₄N₃O₃ |
| 45 | 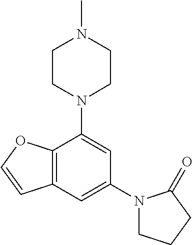 [SMILES: CN1CCN(c2cc(N3CCCC3=O)cc3ccoc23)CC1] 1-(7-(4-methylpiperazin-1- yl)benzofuran-5-yl)pyrrolidin-2-one | ¹H NMR (400 MHz, CDCl3) δ 7.55 (d, J = 2.1, 1H), 7.16 (d, J = 2.0, 1H), 7.13 (d, J = 2.0, 1H), 6.67 (d, J = 2.1, 1H), 3.85 (t, J = 7.0, 2H), 3.48-3.28 (m, 4H), 2.68-2.60 (m, 4H), 2.57 (t, J = 8.1, 2H), 2.34 (s, 3H), 2.12 (dt, J = 7.5, 15.3, 2H). ¹³C NMR (101 MHz, CDCl3) δ 174.26, 144.68, 144.20, 137.38, 135.69, 128.54, 107.33, 106.09, 105.77, 55.30, 50.12, 49.69, 46.37, 32.82, 18.30. | found 300.1716 calculated 300.1712 C₁₇H₂₂N₃O₂ |
| 47a | 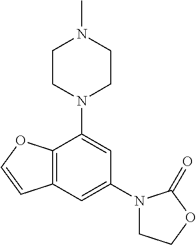 [SMILES: CN1CCN(c2cc(N3CCOC3=O)cc3ccoc23)CC1] 3-(7-(4-methylpiperazin-1- yl)benzofuran-5-yl)oxazolidin-2-one | ¹H NMR (500 MHz, CDCl3) δ 7.57 (d, J = 2.1, 1H), 7.10 (dd, J = 2.0, 15.7, 2H), 6.68 (d, J = 2.1, 1H), 5.26 (s, 1H), 4.53- 4.35 (m, 2H), 4.12-3.99 (m, 2H), 3.38 (s, 4H), 2.70-2.55 (m, 4H), 2.35 (s, 3H). ¹³C NMR (126 MHz, CDCl3) δ 155.91, 144.93, 143.91, 137.62, 134.67, 128.72, 107.28, 104.14, 104.13, 61.44, 55.27, 49.64, 46.51, 46.37. | found 302.1520 calculated 302.1505 C₁₆H₂₀N₃O₃ |
| 47b | 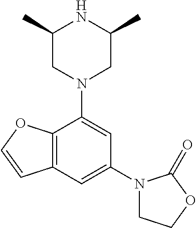 [SMILES: C[C@@H]1CN(c2cc(N3CCOC3=O)cc3ccoc23)C[C@H](C)N1] 3-(7-((3S,5R)-3,5-dimethylpiperazin-1- yl)benzofuran-5-yl)oxazolidin-2-one | ¹H NMR (400 MHz, CDCl3) δ 7.55 (d, J = 2.0, 1H), 7.05 (d, J = 2.6, 2H), 6.66 (d, J = 2.1, 1H), 4.41 (dd, J = 7.2, 8.6, 2H), 4.03 (t, J = 8.0, 2H), 3.71 (d, J = 12.0, 2H), 3.22-3.01 (m, 2H), 2.35 (t, J = 10.9, 2H), 2.12 (s, 1H), 1.10 (d, J = 6.3, 6H). ¹³C NMR (101 MHz, CDCl3) δ 155.91, 144.75, 143.80, 137.44, 134.41, 128.56, 107.10, 104.24, 104.13, 61.34, 56.40, 50.66 (C13), 46.41, 19.55. | found 316.1675, calculated 316.1661 C₁₇H₂₂N₃O₃ |
| 49a | 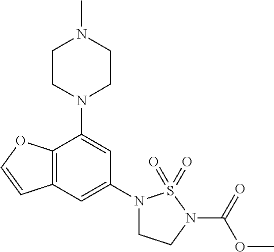 [SMILES: COC(=O)N1CCN(c2cc(N3CCN(C)CC3)c3occc3c2)S1(=O)=O] methyl 5-(7-(4-methylpiperazin-1- yl)benzofuran-5-yl)-1,1-dioxo-1,2,5- thiadiazolidine-2-carboxylate | ¹H NMR (400 MHz, CDCl3) δ 7.60 (d, J = 2.1, 1H), 7.13 (d, J = 2.1, 1H), 6.79 (d, J = 2.0, 1H), 6.72 (d, J = 2.2, 1H), 3.99 (t, J = 6.4, 2H), 3.91 (s, 3H), 3.84 (t, J = 6.4, 2H), 3.46-3.35 (m, 4H), 2.72-2.56 (m, 4H), 2.36 (d, J = 8.3, 3H). ¹³C NMR (101 MHz, CDCl3) δ 151.47, 145.73, 145.26, 138.14, 132.18, 129.25, 108.98, 108.13, 107.40, 55.18, 54.63, 49.38, 46.29, 45.23, 42.71. | found 395.1405, calculated 395.1389 C₁₇H₂₃N₄O₅S |
| 50a | 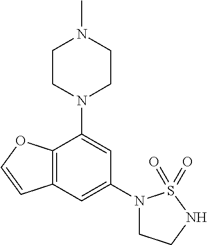 [SMILES: CN1CCN(c2cc(N3CCNS3(=O)=O)cc3ccoc23)CC1] 2-(7-(4-methylpiperazin-1- yl)benzofuran-5-yl)-1,1-dioxo-1,2,5- thiadiazolidine | ¹H NMR (500 MHz, CDCl3) δ 7.58 (d, J = 2.1, 1H), 7.05 (d, J = 2.1, 1H), 6.76 (d, J = 2.1, 1H), 6.69 (d, J = 2.2, 1H), 3.92 (t, J = 6.4, 2H), 3.65 (t, J = 6.4, 2H), 3.48-3.28 (m, 4H), 2.76-2.55 (m, 4H), 2.37 (s, 3H), 1.79 (s, 1H,). ¹³C NMR (126 MHz, CDCl3) δ 145.05, 144.76, 138.00, 133.72, 129.18, 107.35, 106.11, 105.82, 55.23, 49.87, 49.49, 46.32, 39.97. | found 337.1349 calculated 337.1334 C₁₇H₂₂N₃O₃ |
| 59 | 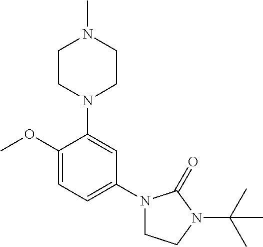 [SMILES: COc1ccc(N2CCN(C(C)(C)C)C2=O)cc1N1CCN(C)CC1] 1-tert-butyl-3-(4-methoxy-3-(4- methylpiperazin-1- yl)phenyl)imidazolidin-2-one | ¹H NMR (500 MHz, CDCl3) δ 7.28 (d, J = 2.6, 1H), 6.91 (dd, J = 2.6, 8.8, 1H), 6.76 (d, J = 8.8, 1H), 3.80 (s, 3H), 3.63 (dd, J = 6.7, 8.7, 2H), 3.43 (dd, J = 6.6, 9.0, 2H), 3.16 (s, 4H), 2.71 (s, 4H), 2.40 (s, 3H), 1.38 (s, 9H). ¹³C NMR (126 MHz, CDCl3) δ 158.55, 148.03, 141.15, 134.85, 112.29, 111.55, 110.08, 55.85, 55.27, 53.62, 50.19, 45.91, 42.88, 40.30, 27.71. | found 347.2445, calculated 347.2447 C₁₉H₃₁N₄O₂ |
| 79 | 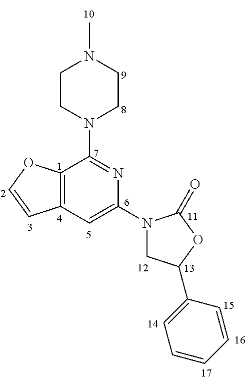 [SMILES: CN1CCN(c2nc(N3CC(c4ccccc4)OC3=O)cc3ccoc23)CC1] 3-[7-(4-Methylpiperazin-1-yl)furo[2,3- c]pyridin-5-yl]-5-phenyl-1,3-oxazolidin- 2-one | 1H NMR: δH (500 MHz, CDCl₃) 7.77 (1H, s, H5), 7.60 (1H, d, J 2.0 Hz, H2), 7.48-7.36 (5H, m, H15, H16, H17), 6.72 (1H, d, J 2.0 Hz, H3), 5.61 (1H, app t, J 8.5 Hz, H13), 4.66 (1H, dd, J 10.5, 8.5 Hz, H12a), 4.15 (1H, dd, J 10.5, 8.0 Hz, H12b), 3.88 (4H, app s, H8), 2.61 (4H, app s, H9), 2.39 (3H, s, H10); 13C NMR: δC (125 MHz, CDCl₃) 154.6 (C11), 146.6 (C2), 143.2 (C), 142.8 (C), 138.5 (C), 137.5 (C), 137.4 (C), 128.9 (C15 and C17), 125.9 (C16) 107.1 (C3), 95.2 (C5), 74.5 (C13), 54.8 (C9), 52.3 (C8), 46.0 (C10), 45.7 (C12); | found 379.1776 calculated 379.1770 C₂₁H₂₃N₄O₃ |
| 80 | 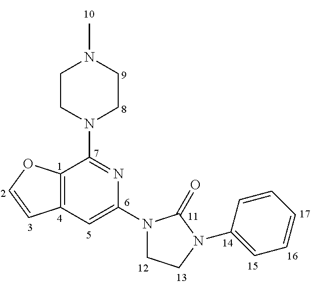 [SMILES: CN1CCN(c2nc(N3CCN(c4ccccc4)C3=O)cc3ccoc23)CC1] 1-(7-(4-Methylpiperazin-1-yl)furo[2,3- c]pyridin-5-yl)-3-phenylimidazolidin- 2-one | 1H NMR: δH (500 MHz, CDCl₃) 7.86 (1H, s, H5), 7.62 (2H, dd, J 8.5, 1.0 Hz, H15), 7.58 (1H, d, J 2.0 Hz, H2), 7.35 (2H, dd, J 8.5, 7.5 Hz, H16), 7.09 (1H, tt, J 7.5, 1.0 Hz, H17), 6.71 (1H, d, J 2.0 Hz, H3), 4.24- 4.18 (2H, m, H12 or H13), 3.98 (4H, br s, H9), 3.96-3.90 (2H, m, H12 or H13), 2.75 (4H, br s, H8), 2.49 (3H, s, H10); 13C NMR: δC (125 MHz, CDCl₃) 154.9 (C11), 146.4 (C2), 144.5 (C), 140.2 (C), 137.6 (C), 137.1 (C), 128.8 (C16), 122.9 (C17), 118.1 (C15), 107.1 (C3), 95.6 (C5), 54.6 (C9), 45.6 (C8), 41.9 (C12 or C13), 41.2 (C12 or C13), 29.7 (C10); | found 378.1941 calculated 378.1930 C₂₁H₂₄N₅O₂ |
| 81 | 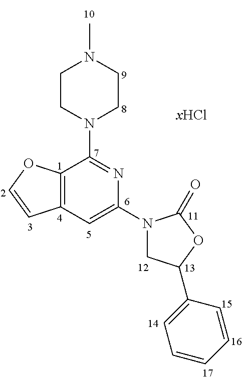 [SMILES: CN1CCN(c2nc(N3CC(c4ccccc4)OC3=O)cc3ccoc23)CC1] 3-[7-(4-Methylpiperazin-1-yl)furo[2,3- c]pyridin-5-yl]-5-phenyl-1,3-oxazolidin- 2-one hydrochloride | 1H NMR*: δH (500 MHz, d6- DMSO) 10.22 (1H, br s, NH), 8.09 (1H, d, J 2.0 Hz, H2), 7.71 (1H, s, H5), 7.50-7.36 (5H, m, H15, H16, H17), 7.02 (1H, d, J 2.0 Hz, H3), 5.73 (1H, app t, J 8.0 Hz, H13), 4.64 (1H, dd, J 10.5, 9.0 Hz, H12a), 4.60 (2H, app br d, J 15.0 Hz, H8a), 4.04 (1H, app dd, J 10.5, 7.5 Hz, H12b), 3.45 (2H, app br d, J 13.0 Hz, H8b) 3.14-3.04 (2H, m, H9a), 2.77 (3H, app d, J 5.0 Hz, H10); 13C NMR: δC (125 MHz, d6- DMSO) 154.0 (C11), 148.8 (C2), 142.9 (C), 141.8 (C), 138.6 (C), 137.8 (C), 136.5 (C), 129.0 (C15, C16 or C17), 128.9 (C15, C16 or C17), 126.4 (C15, C16 or C17), 107.2 (C3), 95.1 (C5), 74.2 (C13), 52.3, 51.9 (C12), 43.4, 43.3, 42.7 (C10). *Note: 1H NMR signal for H9b (approx. 2.51 ppm) obscured by signal for DMSO and is not reported. | |
| 82 | 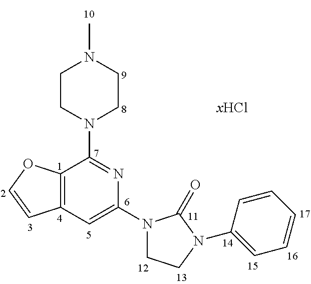 [SMILES: CN1CCN(c2nc(N3CCN(c4ccccc4)C3=O)cc3ccoc23)CC1] 1-(7-(4-Methylpiperazin-1-yl)furo[2,3- c]pyridin-5-yl)-3-phenylimidazolidin-2- one hydrochloride | 1H NMR: δH (500 MHz, d6- DMSO) 10.58 (2H, br s, NH), 8.06 (1H, d, J 2.0 Hz, H2), 7.81 (1H, s, H5), 7.62 (2H, d, J 8.5 Hz, H15), 7.35 (2H, dd, J 8.5, 7.5 Hz, H16), 7.05 (1H, t, J 7.5 Hz, H17), 6.97 (1H, d, J 2.0 Hz, H3), 4.65 (2H, app d, J 14.0 Hz, H8a), 4.15 (2H, t, J 8.0 Hz, H12 or H13), 3.96 (2H, t, J 8.0 Hz, H12 or H13), 3.53 (2H, app d, J 11.5 Hz, H8b), 3.42 (2H, app t, J 13.0 Hz, H9a), 3.14 (2H, app ddd, J 14.0, 12.0, 3.0 Hz, H9b), 2.79 (3H, app d, J 4.5 Hz, H10); 13C NMR: δC (125 MHz, d6- DMSO) 154.0 (C11), 148.4 (C2), 144.3(C), 141.5(C), 140.0 (C), 137.5 (C), 136.0 (C), 128.6 (C5), 122.5 (C3), 117.7 (C15), 107.0 (C16), 95.1 (C17), 51.8, 42.9, 42.1, 41.2 (C12 or C13), 40.8(C12 or C13). | |
| 83 | 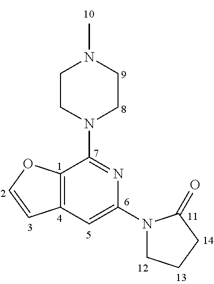 [SMILES: CN1CCN(c2nc(N3CCCC3=O)cc3ccoc23)CC1] 1-[7-(4-Methylpiperazin-1-yl)furo[2,3- c]pyridin-5-yl]pyrrolidin-2-one | 1H NMR: δH (500 MHz, CDCl₃) 7.88 (1H, s, H5), 7.53 (1H, d, J 2.0 Hz, H2), 6.65 (1H, d, J 2.0 Hz, H3), 4.07 (2H, app t, J 7.0 Hz, H12), 3.81-3.79 (4H, m, H8), 2.58 (2H, app t, J 8.0 Hz, H14), 2.52-2.49 (4H, m, H9), 2.29 (3H, s, H10), 2.03 (2H, app quintet, J 7.5 Hz, H13) 13C NMR: δC (126 MHz, CDCl₃) 174.1 (C11), 146.2 (C2) 143.9 (C), 143.3 (C), 137.6 (C), 137.03 (C), 107.1 (C3), 96.9 (C5), 55.0 (C9), 48.0 (C8), 46.3 (C10), 46.1 (C12), 33.8 (C14), 176 (C13); | found 301.1678 calculated 301.1665 C₁₆H₂₁N₄O₂ |
| 84 | 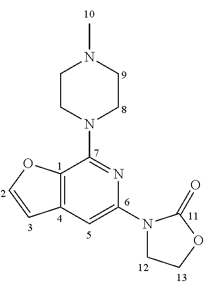 [SMILES: CN1CCN(c2nc(N3CCOC3=O)cc3ccoc23)CC1] 3-[7-(4-Methylpiperazin-1-yl)furo[2,3- c]pyridin-5-yl]oxazolidin-2-one | 1H NMR: δH (500 MHz, CDCl₃) 7.72 (1H, app s, H5), 7.59 (1H, d, J 2.0 Hz, H2), 6.71 (1H, d, J 2.0 Hz, H3), 4.46-4.43 (2H, m, H13), 4.31-4.28 (2H, m, H12), 3.90-3.88 (4H, m, H8), 2.65-2.63 (4H, m, H9), 2.41 (3H, s, H10); 13C NMR: δC (126 MHz, CDCl₃) 155.2 (C11), 146.6 (C2), 143.2 (C), 142.9 (C), 137.5 (C), 137.4 (C), 107.1 (C3), 95.1 (C5), 61.7 (C13), 54.9 (C9), 46.0 (C10), 45.8 (C8), 44.7 (C12); | found 303.1468 calculated 303.1457 C₁₅H₁₉N₄O₃ |

Further Compounds of Formula (I)—Examples a-k

The compounds of Formula (I) listed below may be prepared according to synthetic procedures analogous to those described above.

TABLE 4

| Ex- | | |
|---|---|---|
| ample | Name | Structure |
| a | 3-(4-(4- methylpiperazin- 1-yl) benzofuran- 6-yl) oxazolidin- 2-one | 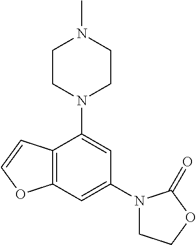 [SMILES: CN1CCN(c2cc(N3CCOC3=O)cc3occc23)CC1] |
| b | 3-(4-(4- methylpiperazin- 1-yl)furo[3,2-c] pyridin-6-yl) oxazolidin- 2-one | 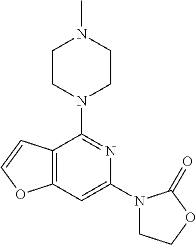 [SMILES: CN1CCN(c2nc(N3CCOC3=O)cc3occc23)CC1] |
| c | 2-methyl-5- [4-(4- methylpiperazin- 1-yl)-1- benzofuran- 6-yl]-1λ⁶,2,5- thiadiazolidine- 1,1-dione | 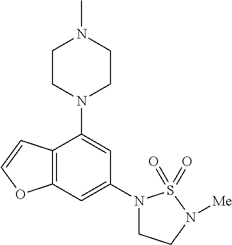 [SMILES: CN1CCN(c2cc(N3CCN(C)S3(=O)=O)cc3occc23)CC1] |
| d | 2-(2- hydroxy- propanoyl)- 5-[7-(4- methylpiperazin- 1-yl)-1- benzofuran-5- yl]-1λ⁶,2,5- thiadiazolidine- 1,1-dione | 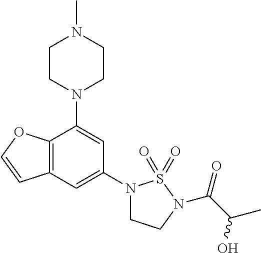 [SMILES: CC(O)C(=O)N1CCN(c2cc(N3CCN(C)CC3)c3occc3c2)S1(=O)=O] |
| e | 2-acetyl-5- [7-(4- methylpiperazin- 1-yl)- 1-benzofuran- 5-yl]-1λ⁶,2,5- thiadiazolidine- 1,1-dione | 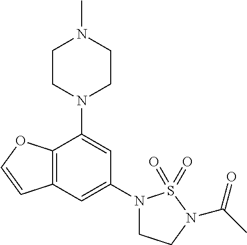 [SMILES: CC(=O)N1CCN(c2cc(N3CCN(C)CC3)c3occc3c2)S1(=O)=O] |
| f | 3-(4-(4- methylpiperazin- 1-yl)benzo[b] thiophen-6-yl) oxazolidin- 2-one | 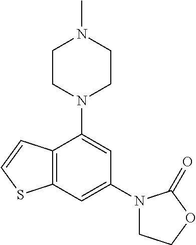 [SMILES: CN1CCN(c2cc(N3CCOC3=O)cc3sccc23)CC1] |
| g | 1-methyl- 3-(4-(4- methylpiperazin- 1-yl)benzo[b] thiophen-6-yl) imidazolidin 2-one | 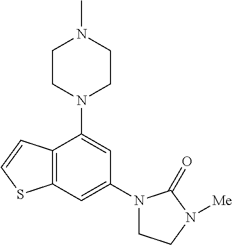 [SMILES: CN1CCN(c2cc(N3CCN(C)C3=O)cc3sccc23)CC1] |
| h | 2-methyl-5- [4-(4- methylpiperazin- 1-yl)-1- benzothiophen- 6-yl]-1λ⁶,2,5- thiadiazolidine- 1,1-dione | 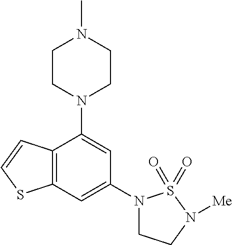 [SMILES: CN1CCN(c2cc(N3CCN(C)S3(=O)=O)cc3sccc23)CC1] |
| i | 1-(4-methoxy- 3-(4- methylpiperazin- 1-yl)phenyl)-4,4- dimethyl- imidazolidin- 2-one | 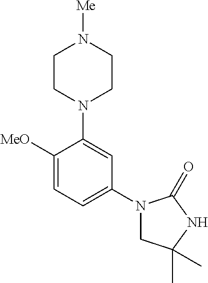 [SMILES: COc1ccc(N2CC(C)(C)NC2=O)cc1N1CCN(C)CC1] |
| j | 2-(7-(4- methylpiperazin- 1-yl)-2,3- dihydro- benzofuran- 5-yl)-1,1- dioxothiazolidine | 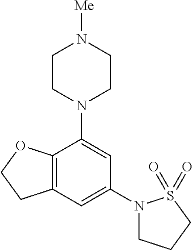 [SMILES: CN1CCN(c2cc(N3CCCS3(=O)=O)cc3c2OCC3)CC1] |
| k | 1-phenyl-3- (7-(piperazin- 1-yl)furo[2,3 c]pyridin-5-yl) imidazolidin- 2-one | 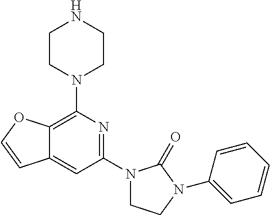 [SMILES: O=C1N(c2ccccc2)CCN1c1cc2ccoc2c(N2CCNCC2)n1] |
| l | 1-(7-((3R,5S)- 3,5- dimethyl- piperazin- 1-yl)furo [2,3-c]pyridin- 5-yl)-3- phenyl- imidazolidin- 2-one | 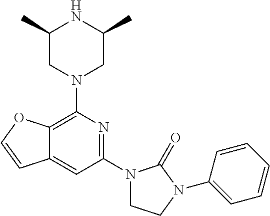 [SMILES: C[C@@H]1CN(c2nc(N3CCN(c4ccccc4)C3=O)cc3ccoc23)C[C@H](C)N1] |
| m | 1-(4- methoxyphenyl)- 3-(7-(4- methylpiperazin- 1-yl)furo[2,3- c]pyridin-5-yl) imidazolidin- 2-one | 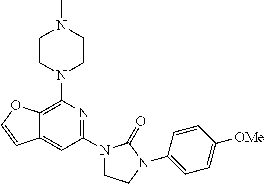 [SMILES: COc1ccc(N2CCN(c3cc4ccoc4c(N4CCN(C)CC4)n3)C2=O)cc1] |
| n | 1-(7-(4- methylpiperazin- 1-yl)furo[2,3- c]pyridin-5-yl)-3- (p-tolyl imidazolidin- 2-one | 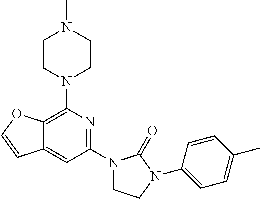 [SMILES: Cc1ccc(N2CCN(c3cc4ccoc4c(N4CCN(C)CC4)n3)C2=O)cc1] |
| o | 1-(4- chlorophenyl)- 3-(7-(4- methylpiperazin- 1-yl)furo[2,3- c]pyridin-5-yl) imidazolidin- 2-one | 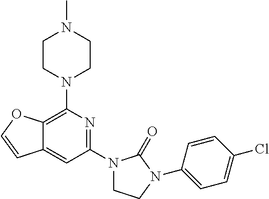 [SMILES: CN1CCN(c2nc(N3CCN(c4ccc(Cl)cc4)C3=O)cc3ccoc23)CC1] |
| p | 1-(3,4- dichlorophenyl)- 3-(7-(4- methylpiperazin- 1-yl)furo[2,3- c]pyridin-5-yl) imidazolidin- 2-one | 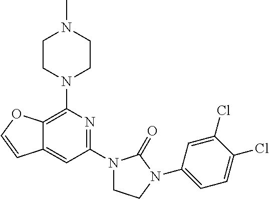 [SMILES: CN1CCN(c2nc(N3CCN(c4ccc(Cl)c(Cl)c4)C3=O)cc3ccoc23)CC1] |
| q | 2-(7-(4- methylpiperazin- 1-yl)furo[2,3- c]pyridin-5-yl)- 5-phenyl-1,2,5- thiadiazolidine 1,1-dioxide | 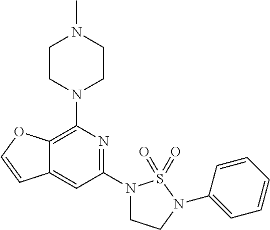 [SMILES: CN1CCN(c2nc(N3CCN(c4ccccc4)S3(=O)=O)cc3ccoc23)CC1] |
| r | 1-(5-methoxy- 6-(4- methylpiperazin- 1-yl)pyridin-2- yl)-3-phenyl- imidazolidin- 2-one | 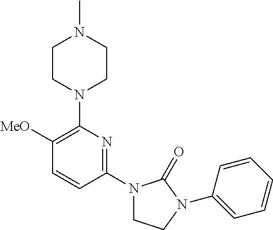 [SMILES: COc1ccc(N2CCN(c3ccccc3)C2=O)nc1N1CCN(C)CC1] |
| s | 1-(5-methoxy- 6-(4- methylpiperazin- 1-yl)pyridin- 2-yl)-3-(4- methoxyphenyl) imidazolidin- 2-one | 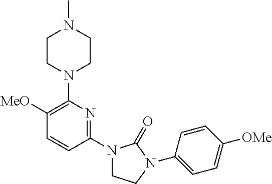 [SMILES: COc1ccc(N2CCN(c3ccc(OC)c(N4CCN(C)CC4)n3)C2=O)cc1] |
| t | 1-(4- chlorophenyl)- 3-(5-methoxy 6-(4- methylpiperazin- 1-yl)pyridin-2-yl) imidazolidin- 2-one | 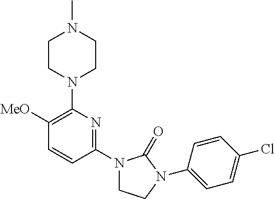 [SMILES: COc1ccc(N2CCN(c3ccc(Cl)cc3)C2=O)nc1N1CCN(C)CC1] |
| u | 1-(4-methoxy- 3-(4-methyl- piperazin-1-yl) phenyl)-3- phenyl- imidazolidin- 2-one | 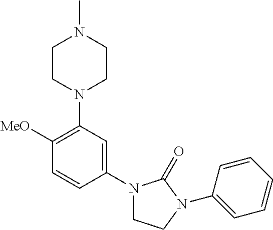 [SMILES: COc1ccc(N2CCN(c3ccccc3)C2=O)cc1N1CCN(C)CC1] |
| v | 1-(4- chlorophenyl)- 3-(4-methoxy- 3-(4- methylpiperazin- 1-yl)phenyl) imidazolidin- 2-one | 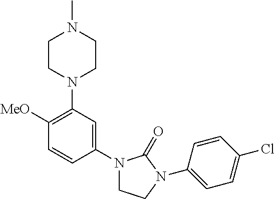 [SMILES: COc1ccc(N2CCN(c3ccc(Cl)cc3)C2=O)cc1N1CCN(C)CC1] |
| w | 2-(5-methoxy- 6-(4- methylpiperazin- 1-yl)pyridin-2- yl)-5-phenyl- 1,2,5- thiadiazolidine 1,1-dioxide | 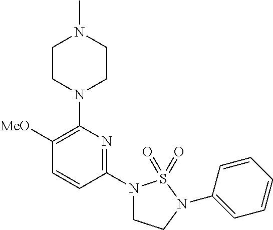 [SMILES: COc1ccc(N2CCN(c3ccccc3)S2(=O)=O)nc1N1CCN(C)CC1] |
| x | 2-(4- chlorophenyl)- 5-(5-methoxy- 6-(4- methylpiperazin- 1-yl)pyridin- 2-yl)-1,2,5- thiadiazolidine 1,1-dioxide | 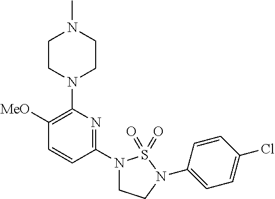 [SMILES: COc1ccc(N2CCN(c3ccc(Cl)cc3)S2(=O)=O)nc1N1CCN(C)CC1] |
| y | 2-(5-methoxy- 6-(4- methylpiperazin- 1-yl)pyridin- 2-yl)-5-(4- methoxyphenyl)- 1,2,5- thiadiazolidine 1,1-dioxide | 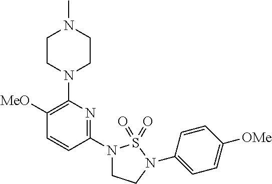 [SMILES: COc1ccc(N2CCN(c3ccc(OC)c(N4CCN(C)CC4)n3)S2(=O)=O)cc1] |

Biological Assays

The activity of compounds according to the invention can be assessed by the following assays:

Binding Protocol for Determination of Binding Affinity at Human h5-H7′₁₈ Receptors (HBA)

Membrane preparations (5 μg in a volume of 100 μl per sample) expressing the human h5-HT_{1B} receptor were preincubated at 27° C. in buffer (50 mM Tris HCl, 10 mM MgCl₂ and 1 mM EDTA; pH 7.4) with or without 10 μM SB214461 (N-(3-(2-(dimethylamino)ethoxy)-4-methoxyphenyl)-2′-methyl-4′-(5-methyl-1,2,4-oxadiazol-3-yl)biphenyl-4-carboxamide, Eur. J. Pharmacol. 1997, 331, 169-174) (to determine non-specific binding). Receptor binding was determined by incubation at 27° C. with 3.5 nM [N-methyl-³H] GR125743 (GE Life Science Products) for 90 min. The incubations were terminated by rapid vacuum filtration through GF/B glass fibre filters that had been presoaked in 3% polyethylenimine. Samples were washed 3 times with 1.5 ml ice-cold buffer (50 mM Tris-HCl, pH 7.4) and bound radioactivity determined by liquid scintillation counting after leaving the filters in contact with the scintillation fluid (4 ml Quicksafe ‘A’, Zinsser, Maidenhead, UK) for at least 4 h before counting for 5 min in a liquid scintillation analyzer.

Specific binding was determined as B—B_{NS}/(B_{Tot}—B_{NS}) where B is the binding in the presence of a given competing ligand, B_{NS} is the non-specific binding of radioligand (i.e. the binding in the presence of 10 μM SB214461), and B_{Tot} is the amount of binding of radioligand in the absence of a competing ligand. Data for specific binding as a function of the concentration of competing ligand were fitted to a single-site model to obtain a value for 10₅₀. Kd values were derived from IC₅₀ by the Cheng & Prusoff equation (Cheng Y, Prusoff W H (1973). Biochem Pharmacol 22, 3099-3108).

Binding Protocol for Determination of Affinity at rat r5-HT _{1B} Receptors (RBA)

This binding affinity protocol was performed according to standard conditions disclosed in Eur. J. Pharmacol. 1985, 118, 1-12.

Protocol for Determination of Efficacy at gp5-HT_{1B} Receptors in the Guinea-Pig Iliac Artery. (GPI)

Guinea-pig common iliac artery segments (1.0-1.5 mm long) from Dunkin-Harley guinea-pigs (250 g-500 g) were mounted under normalized tension in oxygenated (95% O₂; 5% CO₂) Krebs-Henseleit solution (NaCl, 118 mM; KCl, 4.7 mM; MgSO₄, 1.2 mM; KH₂PO₄, 1.2 mM; NaHCO₃, 25 mM; CaCl₂, 2.5 mM; D-glucose, 11 mM; and with indomethacin, 10 μM). After 30 min equilibration, the vessels were precontracted with 5-HT (10 μM) and tested for endothelial integrity by administration of carbachol (10 μM), endothelium-intact vessels (relaxation ≧90% to carbachol) were used for the experiments. Concentration/contraction curves to cumulative addition of 5-nonyloxytryptamine (5-NOT; a 5-HT_{1B} receptor-selective agonist) were constructed in 25 mM KCl Krebs-Henseleit solution (standard Krebs-Henseleit solution in which the KCl concentration was increased to 25 mM by equimolar substitution of NaCl). Vessels were incubated with putative antagonists for 30 min before construction of a concentration/contraction curve for 5-NOT. Contractile responses were expressed as a percentage of the tone induced by Krebs-Henseleit solution containing 90 mM KCl after substitution of an equivalent amount of NaCl with KCl in standard Krebs-Henseleit solution. Data were analysed using non-linear procedures by fitting to a logistic equation: E=(Rₘₐₓ.[A]^{nH})/EC₅₀^{nH}+[A]^{nH}), where E is the contraction induced, [A] the concentration of the agonist, Rₘₐₓ the maximal increase in tension induced, n_{H} the slope function and EC₅₀ the concentration of producing half the maximal contractile tone. Potency was assessed using the Gaddum equation: Kₐ=(concentration ratio −1)/[A] where [A] is the concentration of the putative antagonist and Kₐ its affinity constant at the 5-HT_{1B} receptor. Compounds are classified as having antagonism, agonism or no effect at a dose concentration of 10 μM.

Binding Protocol for Determination of Affinity in gp5-HY_{1B} in Guinea-Pig Frontal Cortex Membranes. (GPF)

Guinea-pig frontal cortex membranes were resuspended in a buffer (50 mM Tris-HCl, 4 mM MgCl, 2.5 mM CaCl₂, 1 mM EDTA and 120 mM NaCl pH 7.4) to a final concentration of 5-6 μg protein μl⁻¹. Receptor binding was initiated by the addition of membranes and carried out in a volume of 0.5 ml at 27° C. Non-specific binding was determined by pre-incubation for 15 min with 10 μM SB214461 (N-(3-(2-(dimethylamino)ethoxy)-4-methoxyphenyl)-2′-methyl-4′-(5-methyl-1,2,4-oxadiazol-3-yl)biphenyl-4-carboxamide, Eur. J. Pharmacol. 1997, 331, 169-174). The amount of binding in the presence or absence of a competing ligand was determined by incubation at 27° C. for 60 min with 0.6 nM [N-methyl ³H] GR125743 (GE Life Science Products). Incubation was terminated by rapid vacuum filtration through GF/B glass fibre filters that had been presoaked in 3% polyethylenimine. Samples were washed 3 times with 1.5 ml ice-cold buffer (50 mM Tris-HCl, pH 7.4) and bound radioactivity determined by liquid scintillation counting after leaving the filters in contact with the scintillation fluid for at least 4 h before counting for 5 min in a liquid scintillation analyzer.

Specific binding was determined as B—B_{NS}/(B_{Tot}—B_{NS}) where B is the binding in the presence of a given competing ligand, B_{NS} is the non-specific binding of radioligand (i.e. the binding in the presence of 10 μM SB214461), and B_{Tot} is the amount of binding of radioligand in the absence of a competing ligand. Data for specific binding as a function of the concentration of competing ligand were fitted to a single-site model to obtain a value for IC₅₀. Kd values were derived from IC₅₀ by the Cheng & Prusoff equation (Cheng Y, Prusoff W H (1973). Biochem Pharmacol 22, 3099-3108).

Protocol for the Determination of the Effect on the Development of Hypoxia-Induced Pulmonary Hypertension.

Mice (C57B/6J, male, 2 months) were exposed to 14 days of hypobaric hypoxia (equivalent to 10% O₂) or normoxia, as described in MacLean, M. R. et al. Circulation 2008, 117, 2928-2937 and MacLean, M. R. et al. Circulation 2004,109, 2150-2155. Mice were dosed either with vehicle (dH₂O) or 82 (15 mg/kg/day) for 14 days. Haemodynamic Measurements: Heart rate, right ventricular pressure and systemic arterial pressure were measured and analysed as described in MacLean, M. R. et al. Circulation 2008, 117, 2928-2937 and MacLean, M. R. et al. Circulation 2004,109, 2150-2155. Briefly, right ventricular pressure was measured via transdiaphragmatic right heart catheterisation and systemic arterial pressure was measured via cannulation of the left common carotid artery. Lung Histology: Sagittal sections of lung were elastica-Van Gieson stained and microscopically assessed for the muscularisation of pulmonary arteries (<80 μm external diameter) in a blinded fashion as described in MacLean, M. R. et al. Circulation 2008, 117, 2928-2937 and MacLean, M. R. et al. Circulation 2004,109, 2150-2155. Remodelled arteries were confirmed by the presence of a double elastic laminae.

Lung sections from 5 mice for each group were studied. Approximately 150 arteries from each lung section (˜750 arteries in total for each group) were assessed. Right Ventricular Hypertrophy: Right ventricular hypertrophy (RVH) was assessed by weight measurement of the right ventricular free wall (RV) and left ventricle plus septum (LV+S). The ratio expressed is RV/LV+S. RVH measurements from 6 to 8 mice for each group were assessed. Myography: Small pulmonary arteries (PAs) of ˜350 μm internal diameter (i.d.) were set up on wire myographs as described in MacLean M. R. et al. J Pharmacol Exp Ther. 2005, 313, 539-548. Briefly, PAs from normoxic mice were set up at tensions equivalent to their mean in vivo right ventricular pressure (RVP) (12-15 mmHg), whereas PAs from hypoxic mice were set up at tensions equivalent to the elevated in vivo mean pressures observed after exposure to hypoxia (25-30 mmHg). After a 45-min equilibration period, the response to 50 mM KCl was determined. Cumulative response curves were constructed in the presence and absence of the antagonist which was allowed a 45-min equilibrium period before constructing the curves.

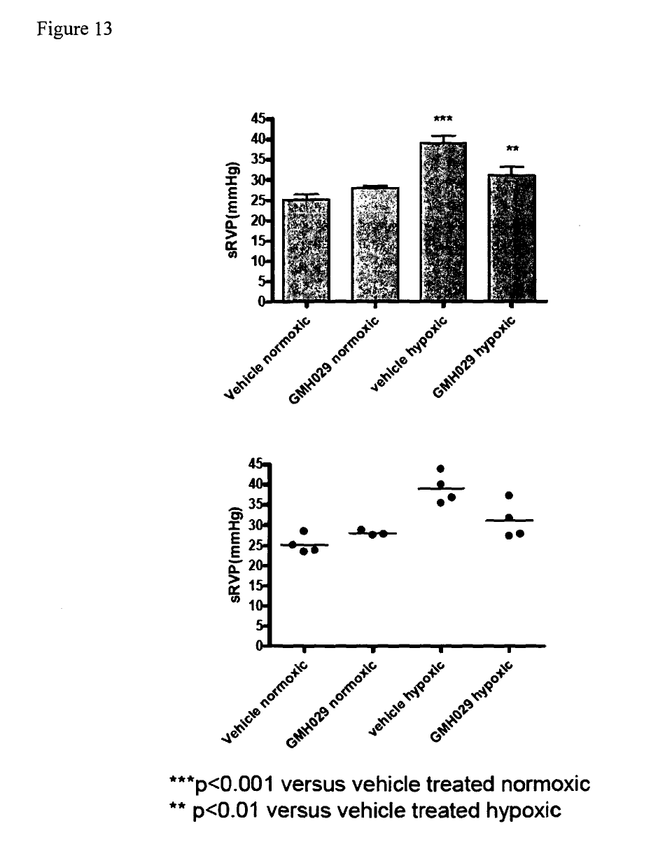

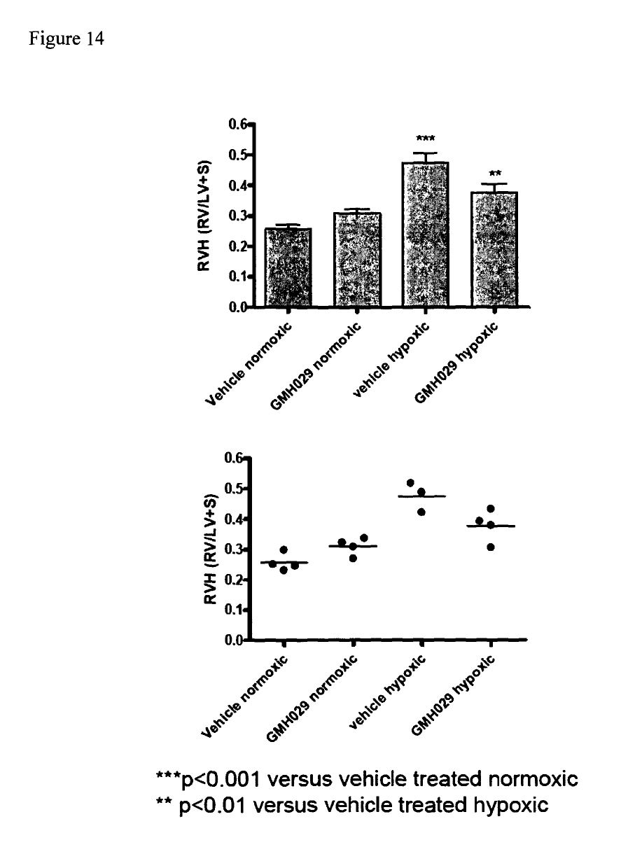

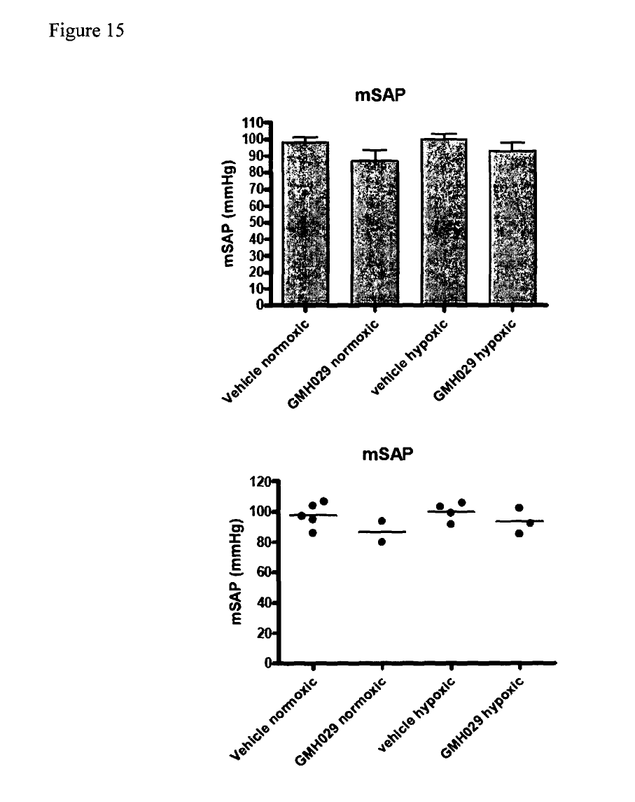

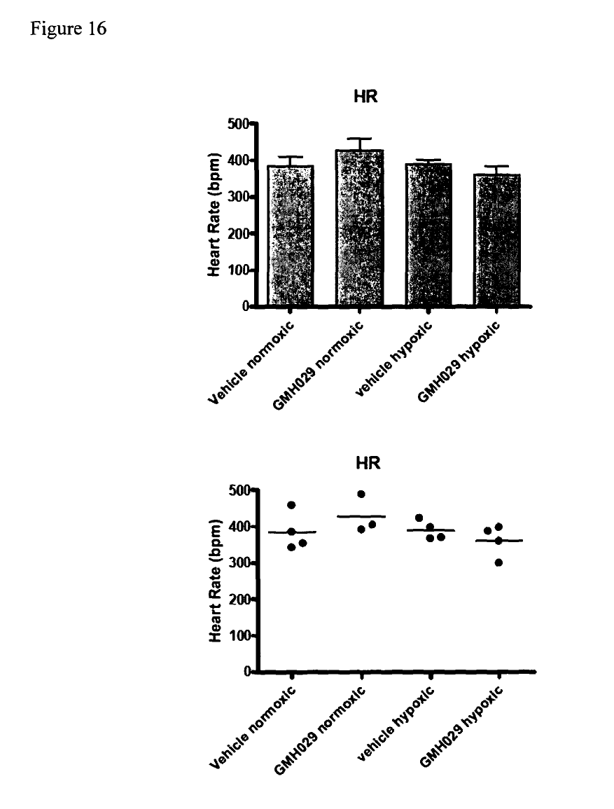

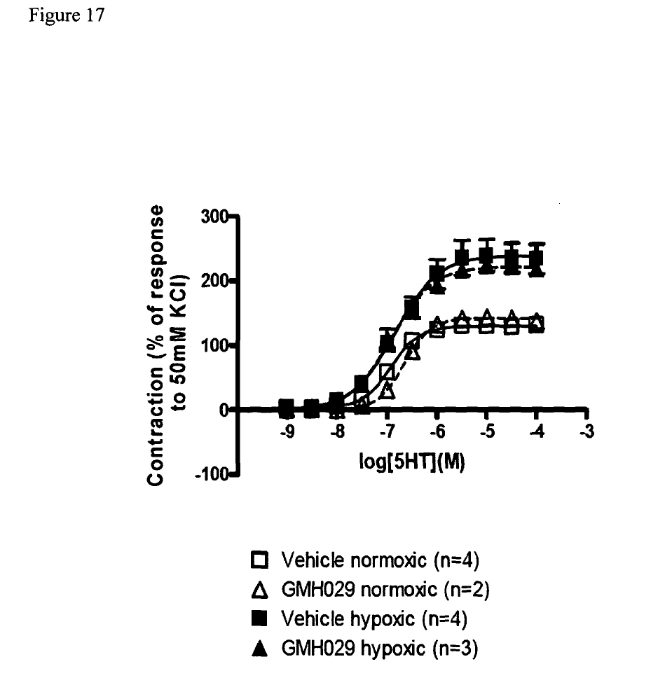

The effects of 82 on a chronically hypoxic murine model of pulmonary arterial hypertension (PAH) were assessed. Four small groups of n=3 or 4 were used. The results are illustrated in FIGS. 13-17 (see also description of figures, above). In summary, 82 (GMH029) at 15mg/kg/day for 14 days significantly attenuated hypoxia-induced increases in systolic right ventricular pressure and right ventricular hypertrophy.

82 (GMH029) at 15 mg/kg/day had no significant effect on mean systemic arterial pressure or heart rate. Furthermore, 82 had no effect on the increase in contractility to 5HT observed in intralobar pulmonary arteries from hypoxic mice.

Table 5 below shows the activity of representative compounds to rat, guinea pig and human 5-HT_{1B} receptors in accordance with the above assay protocols. The data correspond to the monohydrochloride salt of each compound.

TABLE 5

| | | | | (GPF) | | |
|---|---|---|---|---|---|---|
| | (RBA) | | | Guinea-Pig | | (GPF) |
| | Rat | | | Frontal | (GPF) | Guinea-Pig |
| | cerebral | (GPI) | (HBA) | Cortex | Guinea-Pig | Frontal |
| | cortex | Guinea- | Human | membrane | Frontal | Cortex |
| | membrane | Pig Iliac | Binding | binding at | Cortex | membrane |
| | binding at | Artery | Affinity | gp5-HT_{1B} | membrane | binding |
| | r5-HT_{1B} | functional | at | (10 μM | binding | Affinity at |
| Compound | 10 μM | assay at | h5-HT_{1B} | compound | IC₅₀ at | gp5-HT_{1B} |
| No. | (IC₅₀) | gp5-HT_{1B} | K_{d} | concentration) | gp5-HT_{1B} | K_{d} |
| Comparative | 0% | Antagonist | — | 2.5% | — | — |
| Example | | | | | | |
| 13e | | | | | | |
| Comparative | 0% | No effect | — | 27% | — | — |
| Example 13f | | | | | | |
| Example 5a | 61% | Antagonist | — | 47% | 30 μM | 15 μM |
| Example 7 | 24% | | — | 10% | — | — |
| Example | 24% | Antagonist | — | — | — | |
| 13a | | | | | | |
| Example | — | Antagonist | — | — | — | — |
| 13b | | | | | | |
| Example | 33% | Antagonist | — | 15% | — | — |
| 13c | | | | | | |
| Example | 35% | No effect | — | 5% | — | — |
| 13d | | | | | | |
| Example | 44% | — | — | 8% | — | — |
| 18a | | | | | | |
| Example | 50% | — | — | 13% | — | — |
| 18b | (12 μM) | | | | | |
| Example 22 | 10% | — | — | 13% | — | — |
| Example | 33% | — | — | 45% | — | — |
| 26a | | | | | | |
| Example | 54% | — | — | 12% | — | — |
| 26b | (9.6 μM) | | | | | |
| Example | 33% | — | — | 39% | — | — |
| 26c | | | | | | |
| Example 30 | 32% | Antagonist | — | 67% | 4.0 μM | 2.0 μM |
| Example | 39% | — | — | 40% | — | — |
| 33a | | | | | | |
| Example | 33% | — | — | 15% | — | — |
| 33b | | | | | | |
| Example | 27% | — | — | 31% | — | — |
| 33c | | | | | | |
| Example 40 | 37% | — | — | 29% | — | — |
| Example 45 | 84% | Antagonist | 4.5 μM | 75% | 400 nM | 200 nM |
| Example | 84% | Antagonist | 13.5 μM | 84% | 450 nM | 225 nM |
| 47a | | | | | | |
| Example | 93% | — | — | 21.5% | — | — |
| 47b | | | | | | |
| Example | 98% | Antagonist | 1.8 μM | 94% | 460 nM | 230 nM |
| 49a | | | | | | |
| Example | 81% | Antagonist | — | 56% | 1.4 μM | 700 nM |
| 50a | (860 nM) | | | | | |
| Example 82 | 99% | — | — | — | — | — |
| | (110 nM) | | | | | |

### CONCLUSIONS

It can be seen that the compounds of the invention are useful as modulators of 5-HT_{1B} receptors and therefore useful in the treatment of diseases and conditions mediated by 5-HT_{1B} receptors, such as the disorders disclosed herein.

It will be understood that the invention has been described by way of example only and modifications may be made whilst remaining within the scope and spirit of the invention.

1. A compound of formula (I) 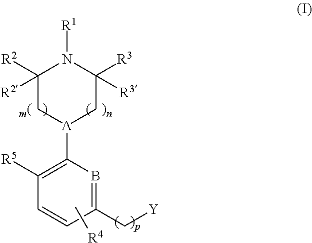
or a pharmaceutically acceptable derivative thereof,
wherein:
A and B are each independently selected from CH and N;
m is 0, 1 or 2;
n is 0, 1 or 2;
p is 0, 1 or 2;
R¹ is H or optionally substituted C₁₋₁₀alkyl, C₃₋₁₀cycloalkyl, C₁-C₁₁heteroalkyl, C₃₋₁₀heterocycloalkyl, C₆₋₁₄aryl or C₅₋₁₄heteroaryl;
R² and R²′ are each independently selected from H and optionally substituted C₁₋₁₀alkyl or C₃₋₁₀cycloalkyl;
R³ and R³′ are each independently selected from H and optionally substituted C₁₋₁₀alkyl or C₃₋₁₀cycloalkyl;
R⁴ is H, NH₂, NO₂, halo, CN or optionally substituted C₁₋₁₀alkyl, C₁₋₁₁heteroalkyl, C₆₋₁₄aryl or C₅₋₁₄heteroaryl;
R⁵ is H, NH₂, NO₂, halo, CN or optionally substituted C₁₋₁₀alkyl, C₁₋₁₁heteroalkyl, C₆₋₁₄aryl or C₅₋₁₄heteroaryl; or R⁵ is taken together with the carbon atom to which it is attached and the adjacent carbon atom to form a 5- or 6-membered ring in a compound according to formula (Ia) or (Ib): 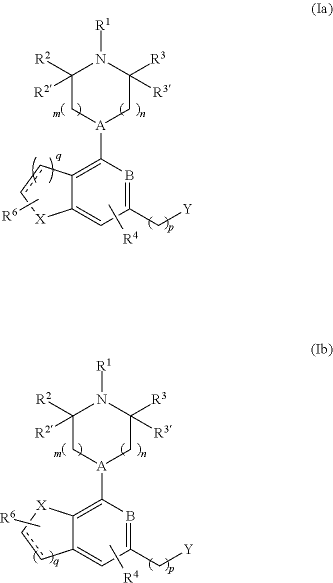
wherein,
X is CH₂, NH, NC₁₋₁₀alkyl, NC(O)C₁₋₁₀alkyl, O or S;
R⁶ is H, NH₂, NO₂, halo, CN or optionally substituted C₁₋₁₀alkyl, C₆₋₁₄aryl or C₅₋₁₄heteroaryl;
q is 1 or 2; and
Y is optionally substituted C₃₋₁₀heterocycloalkyl, C₅₋₁₀heterocycloalkenyl or C₅₋₁₄heteroaryl.

2. The compound of claim 1, wherein:
a) A and B are each N; or b) A is N and B is CH[N],

3. The compound of claim 1, wherein:
a) A is CH and B is N; or
b) A and B are each CH.

4. The compound of claim 1, wherein m is 1 or 2; n is 1 or 2; p is 0 or 1; and/or q is 1.

5. (canceled)

6. (canceled)

7. The compound of claim 1, wherein R¹ is H or optionally substituted C₁₋₁₀alkyl or C₃₋₁₀cycloalkyl.

8. (canceled)

9. (canceled)

10. (canceled)

11. (canceled)

12. (canceled)

13. The compound of claim 1, wherein R⁴ is H, F, Cl, Br, I, NH₂, N(R^{m})₂, CF₃, NO₂, CN, C₁₋₁₀alkyl, C₁₋₁₀alkoxy, C₁₋₁₀alkylamino, C₆₋₁₄aryl, C₅₋₁₄heteroaryl, —OC(O)Rⁿ, C(O)Rⁿ or NHC(O)Rⁿ; wherein each R^{m} is independently selected from C₁₋₄alkyl and C(O)Rⁿ; wherein Rⁿ is C₁₋₄alkyl, C₁₋₄alkoxy or C₁₋₄alkylamino.

14. (canceled)

15. The compound of claim 1, wherein R⁵ is H, F, Cl, Br, I, NH₂, N(R^{s})₂, CF₃, NO₂, CN, C₁₋₁₀alkyl, C₁₋₁₀alkoxy, C₁₋₁₀alkylamino, C₆₋₁₄aryl, C₅₋₁₄heteroaryl, —OC(O)R^{w}, C(O)R^{w} or NHC(O)R^{w}; wherein each R^{s} is independently selected from C₁₋₄alkyl and C(O)R^{w}; wherein R^{w} is C₁₋₄alkyl, C₁₋₄alkoxy or C₁₋₄alkylamino; or
wherein R⁵ is taken together with the carbon atom to which it is attached and the adjacent carbon atom to foul! a 5 or 6 membered ring in a compound of formula (Ia) or (Ib).

16. (canceled)

17. (canceled)

18. The compound of claim 15, wherein R⁵ is taken together with the carbon atom to which it is attached and the adjacent carbon atom to form a 5 or 6 membered ring in a compound of formula (IIa) or (IIb): 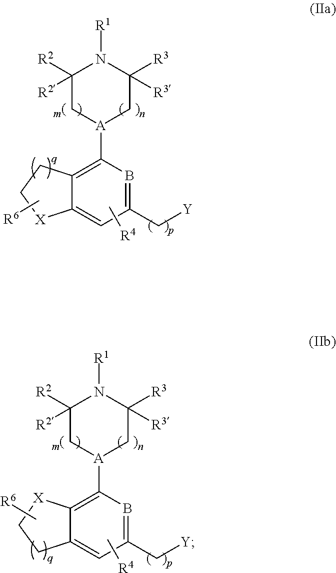
or
wherein R⁵ is a taken together with the carbon atom to which it is attached and the adjacent carbon atom to form a 5 or 6 membered ring in a compound of formula (III) or (IIIb):

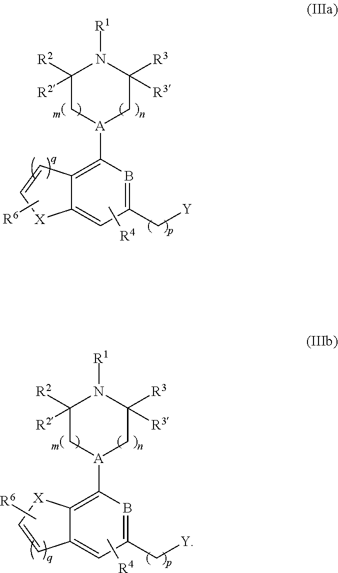

19. (canceled)

20. (canceled)

21. The compound of claim 17, wherein X is O or S.

22. (canceled)

23. (canceled)

24. (canceled)

25. (canceled)

26. (canceled)

27. (canceled)

28. (canceled)

29. (canceled)

30. (canceled)

31. The compound of claim 1, wherein Y is selected from: 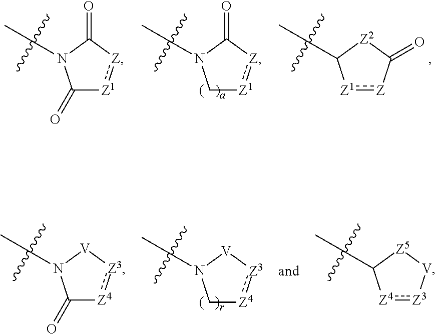
wherein
a and r are independently 0, 1, 2 or 3;
Z is CR⁷ or C(R⁷)₂ and Z¹ is CR⁸ or C(R⁸)₂ or
Z is CR⁷ or C(R⁷)₂ and Z¹ is N, NR⁸, O or S or
Z is N, NR⁷, O or S and Z¹ is CR⁸ or C(R⁸)₂ wherein
each R⁷ and R⁸ is independently selected from H and optionally substituted C₁₋₁₀alkyl, C₃₋₁₀cycloalkyl, C₃₋₁₀heterocycloalkyl, C₅₋₁₀heterocycloalkenyl, C₆₋₁₄aryl and C₅₋₁₄heteroaryl; or R⁷ and R⁸ are taken together with the C or N atoms to which they are attached to form an optionally substituted C₆₋₁₄aryl or C₅₋₁₄heteroaryl moiety;
Z² is CH₂, NH, O or S;
V is S(O)_{y}, wherein
y is 1 or 2;
Z³ is CR⁹ or C(R⁹)₂ and Z⁴ is CR¹⁰ or C(R¹⁰ )₂, or
Z³ is CR⁹ or C(R⁹)₂ and Z⁴ is N, NR¹⁰ or O, or
Z³ is N, NR⁹ or O and Z⁴ is CR¹⁰ or C(R¹⁰), wherein
each R⁹ and R¹⁰ is independently selected from H and optionally substituted C₁₋₁₀alkyl, C₁₋₁₁heteroalkyl, C₃₋₁₀cycloalkyl, C₃₋₁₀heterocycloalkyl, C₅₋₁₀heterocycloalkenyl, C₆₋₁₄aryl and C₅₋₁₄heteroaryl; or R⁹ and R¹⁰ are taken together with the C or N atoms to which they are attached to form an optionally substituted C₆₋₁₄aryl or C₅₋₁₄heteroaryl moiety; and
Z⁵ is CH₂, NH or O.

32. The compound of claim 31, wherein a or r is 1 or 2

33. (canceled)

34. (canceled)

35. The compound of claim 31, wherein Z is N, NR⁷, O or S and Z¹ is CR⁸ or C(R⁸)₂.

36. (canceled)

37. (canceled)

38. (canceled)

39. (canceled)

40. (canceled)

41. (canceled)

42. The compound of claim 31, wherein Z³ is N, NR⁹ or O and Z⁴ is CR¹⁰ or C(R¹⁰)₂.

43. (canceled)

44. (canceled)

45. (canceled)

46. (canceled)

47. The compound of claim 31, wherein each R⁷ and each R⁸ is independently selected from H and optionally substituted C₁₋₁₀alkyl and C₆₋₁₄aryl, and wherein each R⁹ and each R¹⁰ is independently selected from H and optionally substituted C₁₋₁₀alkyl and C₆₋₁₄aryl.

48. (canceled)

49. (canceled)

50. (canceled)

51. The compound of claim 31, wherein when Y is substituted with a group that is itself optionally substituted, the optional substitution is by one or more substituents independently selected from the group consisting of halogen, CF₃, methoxy, methyl, OH, —CO₂H, —SO₂C ₁₋₆alkyl, —C(═O)H, —CSO₂C₁₋₆alkyl, —OSO₂C₆₋₁₄aryl, ═O, —C(═O)NHMe, —NHC(═O)Me, —SO₂NH₂, —SO₂NHC₁₋₆alkyl, —SO₂N(C₁₋₆alkyl)₂, and —SO₂NHC₆₋₁₄aryl.

52. The compound of claim 51, wherein Y is selected from:

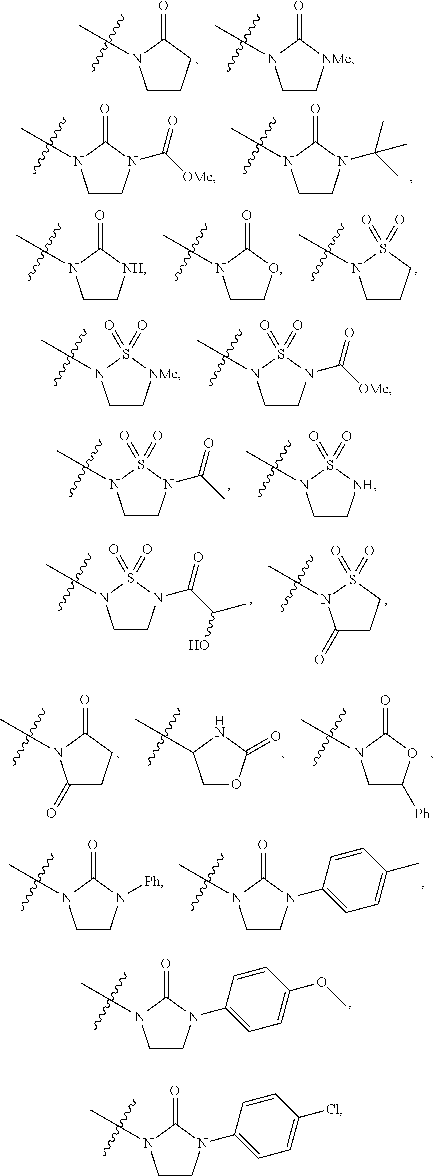

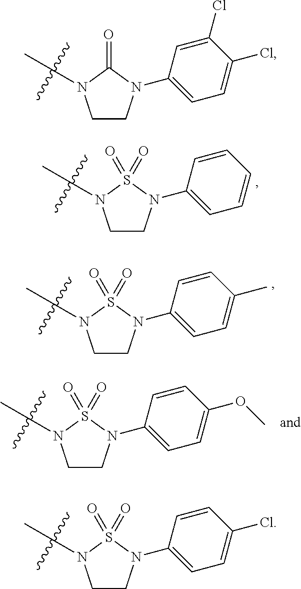

53. The compound of claim 1, wherein the compound is selected from the group consisting of:
1-(3-((3R, 5S)-3,5-dimethylpiperazin-1-yl)-4-methoxyphenyl)pyrrolidin-2-one;
1-(4-methoxy-3-(4-methylpiperazin-1-yl)phenyl)pyrrolidin-2-one;
1-(4-methoxy-3-(4-methylpiperazin-1-yl)phenyl)pyrrolidine-2,5-di one;
3-(3-((3R,5S)-3,5-dimethylpiperazin-1-yl)-4-methoxyphenyl)oxazolidin-2-one;
3-(4-methoxy-3-(4-methylpiperazin-1-yl)phenyl)oxazolidin-2-one;
1-(4-methoxy-3-(4-methyl-1,4-diazepan-1-yl)phenyl)pyrrolidin-2-one;
2-(4-methoxy-3-(4-methyl-1,4-diazepan-1-yl)phenyl)-1,1-di oxoisothiazolidine;
2-(34(3S,5R)-3,5-dimethylpiperazin-1-yl)-4-methoxyphenyl)-1,1-dioxoisothiazolidine;
1-(3-((3S,5R)-3,5-dimethylpiperazin-1-yl)-4-methoxybenzyppyrrolidin-2-one;
1-(7-((3S,5R)-3,5-dimethylpiperazin-1-yl)-2,3-dihydrobenzofuran-5-yl)pyrrolidin-2-one;
2-(3-((3S,5R)-3,5-dimethylpiperazin-1-yl)-4-fluorophenyl)-1,1-dioxoisothiazolidine;
2-(4-fluoro-3-(4-methyl-1,4-diazepan-1-yl)phenyl)-1,1-dioxoisothiazolidine;
2-(4-fluoro-3-(4-methylpiperazin-1-yl)phenyl)-1,1-dioxoisothiazolidine;
1-(7-(4-methylpiperazin-1-yl)-2,3-dihydrobenzofuran-5-yl)pyrrolidin-2-one;
1-(4-fluoro-3-(4-methylpiperazin-1-yl)phenyl)pyrollidin-2-one;
(S)-4-(4-methoxy-3-(4-methylpiperazin-1-yl)benzyl)oxazolidin-2-one;
1-(7-(4-methyl-1,4-diazepan-1-yl)-2,3-dihydrobenzofuran-5-yl)pyrrolidin-2-one;
1-(7-(4-methylpiperazin-1-yl)benzofuran-5-yl)pyrrolidin-2-one;
3-(7-(4-methylpiperazin-1-yl)benzofuran-5-yl)oxazolidin-2-one;
methyl 5-(7-(4-methylpiperazin-1-yl)benzofuran-5-yl)-1,1-dioxo-1,2,5-thiadiazolidine-2-carboxylate;
3-(7-((3S,5R)-3,5-dimethylpiperazin-1-yl)benzofuran-5-ypoxazolidin-2-one;
2-(7-(4-methylpiperazin-1-yl)benzofuran-5-yl)-1,1-dioxo-1,2,5-thiadiazolidine;
1-tert-butyl-3-(4-methoxy-3-(4-methylpiperazin-1-yl)phenyl)imidazolidin-2-one;
3-[7-(4-Methylpiperazin-1-yl)furo[2,3-c]pyridin-5-yl]-5-phenyl-1,3-oxazolidin-2-one;
1-(7-(4-Methylpiperazin-1-yl)furo[2,3-c]pyridin-5-yl)-3-phenylimidazolidin-2-one
3-[7-(4-Methylpiperazin-1-yl)furo[2,3-c]pyridin-5-yl]oxazolidin-2-one
1-[7-(4-Methylpiperazin-1-yl)furo [2,3-c]pyridin-5-yl]pyrrolidin-2-one
3-(4-(4-methylpiperazin-1-yl)benzofuran-6-yl)oxazolidin-2-one;
3-(4-(4-methylpiperazin-1-yl)furo[3,2-c]pyridin-6-yl)oxazolidin-2-one;
3-(7-(4-methylpiperazin-1-yl)furo[2,3-c]pyridin-5-yl)oxazolidin-2-one;
2-methyl-5-[4-(4-methylpiperazin-1-yl)-1-benzofuran-6-yl]-1λ⁶,2,5-thiadiazolidine-1,1-dione;
2-(2-hydroxypropanoyl)-5-[7-(4-methylpiperazin-1-yl)-1-benzofuran-5-yl]-1λ⁶, 2,5-thiadiazolide-1,1-dione;
2-acetyl-5-[7-(4-methylpiperazin-1-yl(-1-benzofuran-5-yl]-1λ⁶, 2,5-thiadiazolide-1,1-dione;
3-(4-(4-methylpiperazin-1-yl)benzo[b]thiophen-6-yl)oxazolidin-2-one;
1-methyl-3-(4-(4-methylpiperazin-1-yl)benzo [b]thiophen-6-yl)imidazolidin-2-one;
2-methyl-5-[4-(4-methylpiperazin-1-yl)-1-benzothiophen-6-yl]-1λ⁶,2,5-thiadiazolidine-1,1-dione;
1-(4-methoxy-3-(4-methylpiperazin-1-yl)phenyl)-4,4-dimethylimidazolidin-2-one;
2-(7-(4-methylpiperazin-1-yl)-2,3-dihydrobenzofuran-5-yl)-1,1-dioxothiazolidine;
1-phenyl-3-(7-(piperazin-1-yl)furo[2,3-c]pyridin-5-yl)imidazolidin-2-one;
1-(7-((3R,5S)-3,5-dimethylpiperazin-1-yl)furo[2,3-c]pyridin-5-yl)-3-phenylimidazolidin-2-one;
1-(4-methoxyphenyl)-3-(7-(4-methylpiperazin-1-yl)furo[2,3-c]pyridin-5-yl)imidazolidin-2-one;
1-(7-(4-methylpiperazin-1-yl)furo[2,3-c]pyridin-5-yl)-3-(p-tolyl)imidazolidin-2-one;
1-(4-chlorophenyl)-3-(7-(4-methylpiperazin-1-yl)furo[2,3-c]pyridin-5-yl)imidazolidin-2-one;
1-(3,4-dichlorophenyl)-3-(7-(4-methylpiperazin-1-yl)furo[2,3-c]pyridin-5-yl)imidazolidin-2-one;
2-(7-(4-methylpiperazin-1-yl)furo[2,3-c]pyridin-5-yl)-5-phenyl-1,2,5-thiadiazolidine 1,1-dioxide;
1-(5-methoxy-6-(4-methylpiperazin-1-yl)pyridin-2-yl)-3-phenylimidazolidin-2-one;
1-(5-methoxy-6-(4-methylpiperazin-1-yl)pyridin-2-yl)-3-(4-methoxyphenyl)imidazolidin-2-one;
1-(4-chlorophenyl)-3-(5-methoxy-6-(4-methylpiperazin-1-yl)pyridin-2-yl)imidazolidin-2-one;
1-(4-methoxy-3-(4-methylpiperazin-1-yl)phenyl)-3-phenylimidazolidin-2-one;
1-(4-chlorophenyl)-3-(4-methoxy-3-(4-methylpiperazin-1-yl)phenyl)imidazolidin-2-one;
2-(5-methoxy-6-(4-methylpiperazin-1-yl)pyridin-2-yl)-5-phenyl-1,2,5-thiadiazolidine 1,1-dioxide;
2-(4-chlorophenyl)-5-(5-methoxy-6-(4-methylpiperazin-1-yl)pyridin-2-yl)-1,2,5-thiadiazolidine 1,1-dioxide;
2-(5-methoxy-6-(4-methylpiperazin-1-yl)pyridin-2-yl)-5-(4-methoxyphenyl)-1,2,5-thiadiazolidine 1,1-dioxide;
and pharmaceutically acceptable derivatives thereof.

54. (canceled)

55. A composition comprising a compound of claim 1 in combination with a pharmaceutically acceptable excipient.

56. (canceled)

57. A method for the treatment of a disease or condition mediated by 5-HT_{1B} receptors, comprising the step of administering a therapeutically effective amount of a compound of a composition of claim 55 to a patient.

58. (canceled)

59. (canceled)

60. (canceled)

61. The method of claim 57, wherein the disease or condition mediated by 5-HT_{1B} receptors is selected from vascular disease, cancer and central nervous system disorders.

62. The method of claim 57, wherein the disease or condition mediated by 5-HT_{1B} receptors is selected from angina, pulmonary hypertension, portal hypertension, Raynaud's syndrome, bladder cancer, prostate cancer, gastrointestinal disorders and chronic obstructive pulmonary disease.

63. (canceled)
